(19) United States
(12) Patent Application Publication
(10) Pub. No.: US 2024/0052342 A1
(43) Pub. Date: Feb. 15, 2024

(54) METHOD FOR DUPLEX SEQUENCING

(71) Applicants: The Broad Institute, Inc., Cambridge, MA (US); Dana-Farber Cancer Institute, Inc., Boston, MA (US)

(72) Inventors: Viktor A. Adalsteinsson, Cambridge, MA (US); Jin Bae, Cambridge, MA (US); Ruolin Liu, Cambridge, MA (US); Gerassimos Makrigiorgos, Boston, MA (US)

(73) Assignees: The Broad Institute, Inc, Cambridge, MA (US); Dana-Farber Cancer Institute, Inc., Boston, MA (US)

(21) Appl. No.: 18/266,566

(22) PCT Filed: Dec. 10, 2021
(86) PCT No.: PCT/US2021/062966

§ 371 Date: Jun. 9, 2023

Related U.S. Application Data
(60) Provisional application No. 63/124,696, filed on Dec. 11, 2020.
(60) Provisional application No. 63/143,334, filed on Jan. 29, 2021.
(60) Provisional application No. 63/208,951, filed on Jun. 9, 2021.
(60) Provisional application No. 63/217,232, filed on Jun. 30, 2021.
(60) Provisional application No. 63/239,920, filed on Sep. 1, 2021.

(51) Int. Cl. C12N 15/10; C12Q 1/6869

(52) U.S. Cl. CPC C12N 15/1093; C12Q 1/6869

(57) ABSTRACT

The disclosure provides a powerful new approach to dual-strand high-throughput next-generation sequencing that improves upon duplex sequencing. The method provides a novel multi-oligonucleotide adapter construct that is ligated to DNA fragments to be sequenced (e.g., genomic DNA fragments) library construction method that concatenates both strands of each DNA duplex into a linear sequence. By physically linking both strands, the products are self-sufficient to form duplex consensus. This strategy has the potential to provide 1,000-fold more accurate sequencing with minimal added cost, and could directly enhance existing products (WGS, WES, targeted panels) offered at the Genomics Platform.

### CROSS REFERENCE TO RELATED APPLICATIONS

This application claims the benefit under 35 U.S.C. § 119(e) of U.S. Provisional Application No. 63/124,696, filed Dec. 11, 2020, entitled “METHOD FOR DUPLEX SEQUENCING,” U.S. Provisional Application No. 63/143,334, filed Jan. 29, 2021, entitled “METHOD FOR DUPLEX SEQUENCING,” U.S. Provisional Application No. 63/208,951, filed Jun. 9, 2021, entitled “METHOD FOR DUPLEX SEQUENCING,” U.S. Provisional Application No. 63/217,232, filed Jun. 30, 2021, entitled “METHOD FOR DUPLEX SEQUENCING,” and U.S. Provisional Application No. 63/239,920, filed Sep. 1, 2021, entitled “METHOD FOR DUPLEX SEQUENCING,” the entire disclosures of each of which are hereby incorporated by reference in their entireties.

### SEQUENCE LISTING

The instant application contains a Sequence Listing which has been submitted in ASCII format via EFS-Web and is hereby incorporated by reference in its entirety. Said ASCII copy, created on Dec. 10, 2021, is named B119570111W000-SEQ-GJM.txt and is 7,934 bytes in size.

### BACKGROUND

DNA is the formative basis of life. Mutations in DNA drive genetic diversity, alter gene function, impact cellular phenotypes, mark cell populations, define evolutionary trajectories, underscore diseases and conditions, and provide targets for precision medicines and diagnostics. Mutations emerge from single cells and are passed to progeny which expand or contract in clonal abundance. It is thus crucial to be able to detect mutations across a wide range of abundances. Detecting low-abundance mutations (e.g. <0.1-1% VAF, down to ‘single duplex’ resolution) is important for studying cancer evolution and drug resistance, understanding somatic mosaicism and clonal hematopoiesis, characterizing base editing technologies such as CRISPR, evaluating the mutagenicity of chemical compounds, uncovering pathogenic variants, studying human embryonic development, detecting microbial or viral infections and cancers and clinically actionable genomic alterations from specimens such as tissue or liquid biopsies, and much more.

In principle, use of third generation “single-molecule” sequencing strategies (e.g., PacBio, Oxford Nanopore Technologies) make it possible to sequence each single DNA duplex in whole to resolve true mutations on both strands apart from false mutations, but in practice lack the required accuracy and throughput. Next generation sequencing (NGS), on the other hand, continues to offer superior read accuracy and throughput, but is not configured to sequence single duplexes at least not without compromising its throughput or utility.

NGS provides high throughput by reading short, clonally amplified DNA fragments in massively parallel fluorescence analysis. Its accuracy, however, is limited by the need to dissociate Watson and Crick strands of each DNA duplex. Without a complementary strand for comparison, errors introduced on either strand due to base damage, PCR, and sequencing (i.e., “false mutations”) can be disguised as real mutations (see e.g., FIG. 1A). While it is possible to use unique molecular identifiers (UMIs) to separately track both strands of each DNA molecule and compare their sequences to distinguish true mutations (which would be present on both strands of each duplex) from false mutations (which would be present only on one of the strands of a duplex), it does not solve the underlying limitation of NGS, namely, duplex dissociation.

A modified NGS workflow called “duplex sequencing” was first described in Schmitt et al., “Detection of ultra-rare mutations by next-generation sequencing,” PNAS, Sep. 4, 2012, Vol. 109, No. 36, pp. 14508-14513 (the entire contents of which are incorporated herein by reference) and was designed overcome the limitations of NGS associated with the sequencing of single-stranded DNA. The method relies on a specialize adapter referred to in Schmitt et al. as the “Duplex Tag,” which is a double-stranded, randomized sequence that is appended to the ends of DNA fragments sandwiched between a DNA fragment and an NGS flow cell adapter prior to proceeding through the NGS workflow (e.g., cluster amplification on flow cell, sequencing to generate sequence reads, and alignment/data analysis). During the analysis stage, sequence reads (which include sequences of both strands of the DNA fragments) are grouped into sets of top and bottom strand sequences of the same DNA fragments by matching the appropriate Duplex Tags. These sets are sequence aligned and compared to generate single-strand consensus sequences (SSCS) representing the consensus sequences for each top and bottom single strand of the sequenced duplexes. At this stage, the SSCS still include true mutations and false mutations. The Duplex Tags are then used to pair the top and bottom strand SSCS to thereby establish a consensus duplex sequence which are then analyzed to sort true mutations from false mutations. Given the inherent informational controls in the top and bottom strands of each DNA duplex, the true mutations are those that appear in both top and bottom strand sequences, whereas the false mutations appear only in one of the strand sequences.

By forming the duplex consensus between reads assigned to the Watson and Crick top and bottom strands of each original duplex, duplex sequencing achieves up to 1,000-fold or higher accuracy and can resolve true mutations from false mutations within single DNA duplexes. However, recovering both strand sequences from among up to 10 billion other strands on an NGS flow cell (e.g., Illumina, NovaSeq) requires 100-fold excess of sequencing reads as compared to standard NGS workflow, which invariably diminishes the throughput of NGS and severely limits the applicability of duplex sequencing in part due to excessive cost. This high inefficiency of duplex sequencing also stems from both strands being separated after adapter ligation and independently amplified during the NGS workflow. This skews the representation of strands and leads to a massive number of reads being required to read both strands at least once.

Accordingly, new methods are needed to improve the accuracy and throughput of dual-strand sequencing methods, such as duplex sequencing, without compromising mutation detection and without requiring a high cost.

### SUMMARY OF THE INVENTION

The present disclosure provides a novel duplex or “dual-strand” sequencing method referred to herein as “Concatenating Original Duplex for Error Correction” sequencing or “CODEC” sequencing which improves upon the shortcomings of traditional duplex sequencing. The method produces high-quality DNA sequencing reads capable of detecting rare mutations while doing so at a low cost.

In various aspects, the disclosure provides methods for CODEC sequencing as well as compositions required for and/or produced by CODEC sequencing, including adapters (referred to herein in various embodiments as “CODEC adapters”), circularized intermediates each comprising a CODEC adapter ligated to both ends of a DNA fragment to be sequenced (referred to herein in various embodiments as “CODEC circularized intermediates”), and linearized double-stranded products comprising concatenated top and bottom strands of the single DNA fragments to be sequenced (referred to herein in various embodiments as “the CODEC library” or individually as “CODEC library members”). In various embodiments, the CODEC adapter includes NGS adapters for NGS workflow (e.g., cluster amplification on NGS flow cell), sequencing read primer sites for reading both strands of a DNA fragment, and optionally one or more sample indices and one or more unique molecular identifiers (UMIs).

Unlike traditional duplex sequencing which separately obtains the top and bottom strand sequences for each DNA fragment to be sequenced (and thus, requiring computational approaches to identify, match, and compare the top and bottom strand sequences), each of the CODEC library members is self-sufficient for forming a duplex consensus sequence in the same read because library formation using the CODEC adapter results in double-stranded library members whereby each strand comprises a concatemer of top and bottom sequences of each original DNA fragment (i.e., in the same DNA molecule) to be sequenced. Thus, sequencing of the CODEC adapter results in a sequencing product that comprises the top strand, the bottom strand, and optionally one or more sample indices and one or more UMIs. The technical advantage of this approach, as compared to standard duplex sequencing, is that unlike standard duplex sequencing which generates two separate sequencing products (i.e., one for the top sequence and one for the bottom sequence), CODEC sequencing results in a single sequencing product that comprises both the top sequence and the bottom sequence thereby allowing a user to easily discern true mutations (mutations that appear in both the top and bottom portions of the sequencing read) from false mutations (mutations that appear in only the top or bottom portion of the sequencing read).

In other aspects, the disclosure describes the read primers for conducting sequencing, as well as methods of sequencing the CODEC library (e.g., by NGS sequencing). The disclosure further provides computer-based methods for analyzing the resulting sequence read information, including, but not limited to analyzing the built-in duplex consensus comprising a concatenated top and a bottom strand sequence read. By comparing the top and bottom sequences of a single read, one is able to discern true mutations (mutations that appear in both the top and bottom portions of the sequencing read) from false mutations (mutations that appear in only the top or bottom portion of the sequencing read).

In still other aspects, the disclosure provides methods and applications for CODEC sequencing, including, but not limited to, methods for sequencing DNA, methods for detecting mutations in DNA, methods for detecting rare or low-abundant mutations in DNA, methods for diagnosing and/or predicting disease based on detection of one or more mutations in DNA, methods of diagnosing and/or predicting a genetic conditions by detection of one more mutations in DNA, and methods of diagnosing and/or predicting a disease or condition by sequencing one or more genes and detecting one or more disease-associated sequences (e.g., a rare mutation). In other aspects, the disclosure provides compositions (e.g., CODEC adapters) and kits for practicing the subject method as described herein.

In yet another aspect, as exemplified in Example 2 and FIG. 16 of the Specification, the disclosure also describes a method for methylation-specific CODEC sequencing which can be used for performing improved mutation and methylation sequencing of DNA samples. In one embodiment, the disclosure provides a method for methylation sequencing (or “methyl-seq”) of a DNA fragment comprising preparing a CODEC adaptor that is modified to contain methylated cytosine in place of unmethylated cytosine, wherein the methylated cytosines are refractory to subsequent deamination and can undergo amplification involved in the CODEC workflow. Next, the modified CODEC adaptors are ligated to the both ends of the DNA fragment, thereby producing a partially circularized, partially double-stranded intermediate construct comprising the CODEC adapter (having available 5′ ends in the central duplex of the CODEC adapter) and the DNA fragment. Next, the available 5′ ends are extended by a DNA polymerase in the presence of methylated-dCTP along with standard dATP, dGTP and dTTP deoxynucleotides, wherein the DNA polymerase uses the opposite strand of the intermediate construct as a template. DNA extension in this way from both available 5′ ends produces a double-stranded product comprising a concatemer of FIG. 1D which includes a first strand that comprises the original top strand of the DNA fragment concatenated with a bottom strand (which is a product of copying the original top sequence) and a second strand that is the reverse complement of the top strand and which comprises the original bottom strand of the DNA fragment concatenated with a top strand (which is a product of copying the original bottom strand). See FIG. 1D. However, in this embodiment, the copied regions are methylated at cytosine positions which are refractory to subsequent de-amination. Next, a deamination step is conducted to convert un-methylated cytosines to uracils in the original DNA strand. In various embodiments, the deamination of cytosines can be performed by any suitable method, such as by bisulfite-de-amination², by enzymatic deamination using enzymatic methyl-seq (EM-seq) technique, which uses enzymatic steps by TET2 and APOBEC2 enzymes to differentiate between methylated and un-methylated cytosine³, or by the TET Assisted Pic-borane Sequencing (TAPS) method⁴. Following the deamination step, amplification using the CODEC adaptor primers is applied, followed by duplex sequencing as otherwise described herein.

One aspect of the present disclosure relates to an isolated nucleic acid complex (complex) comprising at least ten (10) regions (R01-R10) in the following configuration:

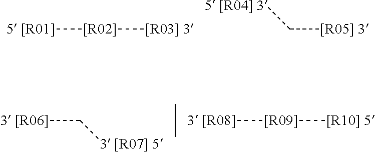

wherein, [symbol] represents bonding, wherein R01, R02, and R03 comprise a first oligonucleotide, wherein R04 and R05 comprise a second oligonucleotide, wherein R06 and R07 comprise a third oligonucleotide, wherein R08, R09, R10 comprise a fourth oligonucleotide, wherein, R01 and R06 are annealed to one another, wherein, R03 and R08 are annealed to one another, wherein, R05 and R10 are annealed to one another, wherein, R02 and R07 are not annealed to one another, and wherein, R04 and R09 are not annealed to one another; wherein R02 comprises a single-stranded linker, first unique molecular identifier (UMI), and a first read primer site, and wherein R09 comprises a single-stranded linker, a second UMI, and a second read primer site.

In some embodiments, R01 comprises a first adapter; R02 comprises a single-stranded linker, first unique molecular identifier (UMI), and a first read primer site; R03 comprises a first sequence at or near the 3′ end capable of priming DNA synthesis by a DNA-dependent DNA polymerase; R04 comprises a free 5′ end comprising a first next-generation sequencing (NGS) adapter sequence; R05 comprises a third adapter and a first sample index; R06 comprises a second adapter and a second sample index; R07 comprises a free 5′ end comprising a second adapter sequence; R08 comprises a second sequence at or near the 3′ end capable of priming DNA synthesis by a DNA-dependent DNA polymerase; R09 comprises a single-stranded linker, a second UMI, and a second read primer site; and/or R10 comprises a fourth adapter.

In some embodiments, each of the four oligonucleotides may be combined before library preparation, thereby forming the complex prior to library preparation. In other embodiments, the four oligonucleotides may each be added separately during library preparation, thereby forming the hybridized complex commensurate or during library preparation.

In some embodiments, the first sequence and second sequence, further comprise the same or different primer binding sites. In some embodiments, the first and second primer sites are oriented to initiate sequencing by addition in opposing directions. In some embodiments, the first and second UMI are distinct.

In some embodiments, R01 comprises at least 12 nucleotides R02 comprises at least 14 nucleotides, R03 comprises at least 12 nucleotides, R04 comprises at least 20 nucleotides, R05 comprises at least 12 nucleotides, R06 comprises at least 12 nucleotides, R07 comprises at least 20 nucleotides, R08 comprises at least 12 nucleotides, R09 comprises at least 14 nucleotides, and/or R10 comprises at least 12 nucleotides. In some embodiments, R01 comprises less than 30 nucleotides, R02 comprises less than 75 nucleotides, R03 comprises less than 99 nucleotides, R04 comprises less than 49 nucleotides, R05 comprises less than 30 nucleotides, R06 comprises less than 30 nucleotides, R07 comprises less than 49 nucleotides, R08 comprises less than 99 nucleotides, R09 comprises less than 75 nucleotides, and/or R10 comprises less than 30 nucleotides. In some embodiments, R01 comprises between 12 and 30 nucleotides, R02 comprises between 14 and 75 nucleotides, R03 comprises between 12 and 99 nucleotides, R04 comprises between 20 and 49 nucleotides, R05 comprises between 12 and 30 nucleotides, R06 comprises between 12 and 30 nucleotides, R07 comprises between 20 and 49 nucleotides, R08 comprises between 12 and 99 nucleotides, R09 comprises between 14 and 75 nucleotides, and/or R10 comprises between 12 and 30 nucleotides.

In some embodiments, R01 and R06 comprise a hybridization free energy of about −10 kcal/mol, about −15 kcal/mol, about −20 kcal/mol, about −25 kcal/mol, about −30 kcal/mol, or about −35 kcal/mol; R03 and R08 comprise a hybridization free energy of about −10 kcal/mol, about −15 kcal/mol, about −20 kcal/mol, about −25 kcal/mol, about −30 kcal/mol, about −35 kcal/mol, about −40 kcal/mol, about −45 kcal/mol, about −50 kcal/mol, about −55 kcal/mol, about −60; and/or R05 and R10 comprise a hybridization free energy of about −10 kcal/mol, about −15 kcal/mol, about −20 kcal/mol, about −25 kcal/mol, about −30 kcal/mol, or about −35 kcal/mol.

In some embodiments, R01 and R06 each comprise the same number of nucleotides, optionally wherein R06 has a one nucleotide overhang to facilitate ligation; R03 and R08 each comprise the same number of nucleotides; and/or R05 and R10 each comprise the same number of nucleotides, optionally wherein R05 has a one nucleotide overhang to facilitate ligation.

In some embodiments, R01 and R06 comprise sequences with at least 90% complementarity; R03 and R08 comprise sequences with at least 90% complementarity; and/or R05 and R10 comprise sequences with at least 90% complementarity. In some embodiments, each R01, R06, R05, and R10 comprise the same number of nucleotides, optionally wherein R06 and R05 each have a one nucleotide overhang to facilitate ligation.

In some embodiments, the complex comprises at least two elements described above. In some embodiments, the complex comprises at least three elements described above. In some embodiments, the complex comprises at least four elements described above. In some embodiments, the complex comprises at least five elements described above. In some embodiments, the complex comprises at least six elements described above. In some embodiments, the complex comprises at least seven elements described above. In some embodiments, the complex comprises at least eight elements described above. In some embodiments, the complex comprises at least nine elements described above.

In some embodiments, R01 comprises a first adapter; R02 comprises a single-stranded linker; R03 comprises a 3′ end capable of priming DNA synthesis by a DNA-dependent DNA polymerase; R04 comprises a first unique molecular identifier (UMI); R05 comprises a third adapter; R06 comprises a second adapter; R07 comprises a second UMI; R08 comprises a 3′ end capable of priming DNA synthesis by a DNA-dependent DNA polymerase; R09 comprises a single-stranded linker; and R10 comprises a fourth adapter.

In some embodiments, the 5′ end of R01 is ligated to the 3′ end of a first strand of a target DNA duplex; the 3′ end of R05 is ligated to the 5′ end of the first strand of the target DNA duplex; the 5′ end of R10 is ligated to the 3′ end of a second strand of the target DNA duplex; the 3′ end of R06 is ligated to the 5′ end of the second strand of the target DNA duplex; forming a circularized DNA duplex or optionally a partially double-stranded circular DNA.

Another aspect of the present disclosure relates to the isolated nucleic acid complex as described herein for use in next-generation sequence of a DNA sample.

Another aspect of the present disclosure relates to the isolated nucleic acid complex as described herein for use in place of a duplex adapter in a next generation sequencing workflow to obtain the sequence of a DNA sample.

Another aspect of the present disclosure relates to a sequencing adapter having a first end, a second end and a central portion positioned between the first and second ends, wherein the first end comprises a first duplex comprising a first oligonucleotide annealed to a second oligonucleotide, wherein the second end comprises a second duplex comprising a third oligonucleotide annealed to a fourth oligonucleotide, and wherein the second and the fourth oligonucleotides are annealed to one another over a region complementarity to form a third duplex that is positioned in the central portion, wherein the sequencing adapter further comprises a pair of read primer binding sites on either side of the third duplex in single stranded regions.

In some embodiments, the first duplex is 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, or 40 bp in length. In some embodiments, the first duplex has hybridization free energy of about −10 kcal/mol, about −15 kcal/mol, about −20 kcal/mol, about −25 kcal/mol, about −30 kcal/mol, or about −35 kcal/mol. In some embodiments, the second duplex is 10 bp, 11 bp, 12 bp, 13 bp, 14 bp, 15 bp, 16 bp, 17 bp, 18 bp, 19 bp, 20 bp, 21 bp, 22 bp, 23 bp, 24 bp, or 25 bp in length. In some embodiments, the first duplex has hybridization free energy of about −10 kcal/mol, about −15 kcal/mol, about −20 kcal/mol, about −25 kcal/mol, about −30 kcal/mol, or about −35 kcal/mol. In some embodiments, the third duplex is 10 bp, 11 bp, 12 bp, 13 bp, 14 bp, 15 bp, 16 bp, 17 bp, 18 bp, 19 bp, 20 bp, 21 bp, 22 bp, 23 bp, 24 bp, or 25 bp in length. In some embodiments, the third duplex has hybridization free energy of about −10 kcal/mol, about −15 kcal/mol, about −20 kcal/mol, about −25 kcal/mol, about −30 kcal/mol, or about −35 kcal/mol.

In some embodiments, the single stranded regions are 5, 6, 7, 8, 9, 10, 11, 12, 1, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the first oligonucleotide comprises a free 5′ end comprising a first next-generation sequencing (NGS) flow cell binding region. In some embodiments, the third oligonucleotide comprises a free 5′ end comprising a second next-generation sequencing (NGS) flow cell binding region. In some embodiments, the first duplex has a first free 5′ end and the second duplex has a second free 5′ end. In some embodiments, the third duplex comprises a free 5′ end on each strand of the duplex. wherein the first and second 3′ ends can prime DNA synthesis by a DNA-dependent DNA polymerase.

Another aspect of the present disclosure relates to the sequencing adapter as described herein for use in next-generation sequence of a DNA sample.

Another aspect of the present disclosure relates to the sequencing adapter as described herein for use in place of a duplex adapter in a next generation sequencing workflow to obtain the sequence of a DNA sample.

Another aspect of the present disclosure relates to a method of preparing a sequencing library, comprising: ligating the complex described herein to a dsDNA duplex as follows: ligating the 5′ end of R01 to the 3′ end of a first strand of the dsDNA duplex; ligating the 3′ end of R05 to the 5′ end of the first strand of the dsDNA duplex; ligating the 5′ end of R10 to the 3′ end of a second strand of the dsDNA duplex; and ligating the 3′ end of R06 to the 5′ end of the second strand of the dsDNA duplex; thereby forming a circular double-stranded DNA intermediate comprising the target DNA molecule and the complex; extending a first DNA strand from the 3′ end of R03; extending a second DNA strand from the 3′ end of R08; and optionally annealing the first and second DNA strands to form a double-stranded DNA molecule for use in next-generation sequencing (NGS) of the target DNA molecule.

In some embodiments, the double-stranded DNA molecule comprises two copies of the target DNA molecule. In some embodiments, the ligating of the first step described above comprises adding ligase. In some embodiments, the synthesizing of the second and third steps described above comprise contacting the circular double-stranded DNA intermediate with a polymerase. In some embodiments, the polymerase is a DNA-dependent DNA polymerase.

In some embodiments, the polymerase has a strand-displacement activity. In some embodiments, the next-generation sequencing (NGS) is a short-read strategy. In some embodiments, the method further comprises sequencing double-stranded DNA molecule by next-generation sequencing.

Another aspect of the present disclosure relates to a method of preparing a sequencing library comprising a plurality of DNA duplexes to be sequenced, comprising for each member of the library: ligating the first and second ends of a sequencing adapter described herein to a sample DNA fragment having opposing top and bottom strands, thereby forming a partially circularized DNA molecule comprising the DNA fragment and the sequencing adapter; and synthesizing first and second single-strand DNA molecules by extending the free 3′ ends on the sequencing adapter each using the opposite strand of the partially circularized DNA molecule as a template, thereby forming a linearized double-stranded DNA molecule configured for next generation sequencing, said linearized double-stranded DNA molecule comprising a first double-stranded region comprising the original top strand paired with a copied bottom strand, and a second double-stranded region comprising a copied top strand paired with the original bottom strand, wherein a plurality of linearized double-stranded DNA molecule each prepared from a different DNA fragment constitute the next-generation sequencing library.

In some embodiments, the linearized double-stranded DNA molecule configured for next generation sequencing and having first and second ends comprises the following structure:

first end-[a first next generation flow cell adapter]-[a first duplex region comprising the original top strand paired with a copy of original bottom strand]-[a second duplex region comprising the central portion of the next-generation sequencing adapter]-[a third duplex region comprising a copy of original top strand paired with the original bottom strand]-[a second next generation flow cell adapter]-second end.

In some embodiments, the first next generation flow cell adapter is an Illumina P5 or P7 adapter sequence. In some embodiments, the second next generation flow cell adapter is an Illumina P5 or P7 adapter sequence. In some embodiments, the second duplex region comprises first and second read primer binding sites, wherein each first and second read primer sites is further associated with a unique molecule identifier (UMI) and a sample index sequence.

In some embodiments, the first and second read primer binding sites are orientated outwardly towards the ends of the linearized double-stranded DNA molecule. In some embodiments, a first read primer can be used to obtain a sequence read comprising a UMI, sample index, and the original top strand, or portion thereof, of the sample DNA fragment to be sequenced. In some embodiments, a second read primer can be used to obtain a sequence read comprising a UMI, sample index, and the original bottom strand, or portion thereof, of the sample DNA fragment to be sequenced. In some embodiments, the method is used in place of a commercial next-generation library construction kit. In some embodiments, the ligating of the first step described above comprises adding ligase. In some embodiments, the synthesizing of the second step described above comprising adding a DNA polymerase. In some embodiments, the polymerase has a strand-displacement activity. In some embodiments, the methods further comprise the step of obtaining the sequence of the original top and original bottom strands by conducting next generation sequencing with the first and second read primers.

Another aspect of the present disclosure relates to a linearized double-stranded DNA molecule configured for next generation sequencing obtained by the method described herein, wherein the linearized double-stranded DNA molecule comprises first and second ends and has the following structure:

first end-[a first next generation flow cell adapter]-[a first duplex region comprising the original top strand paired with a copy of original bottom strand]-[a second duplex region comprising the central portion of the next-generation sequencing adapter]-[a third duplex region comprising a copy of original top strand paired with the original bottom strand]-[a second next generation flow cell adapter]-second end.

In some embodiments, the first next generation flow cell adapter is an Illumina P5 or P7 adapter sequence. In some embodiments, the second next generation flow cell adapter is an Illumina P5 or P7 adapter sequence. In some embodiments, the second duplex region comprises first and second read primer binding sites, wherein each first and second read primer sites is further associated with a unique molecule identifier (UMI) and a sample index sequence. In some embodiments, the first and second read primer binding sites are orientated outwardly towards the ends of the linearized double-stranded DNA molecule. In some embodiments, a first read primer can be used to obtain a sequence read comprising a UMI, sample index, and the original top strand, or portion thereof, of the sample DNA fragment to be sequenced. In some embodiments, a second read primer can be used to obtain a sequence read comprising a UMI, sample index, and the original bottom strand, or portion thereof, of the sample DNA fragment to be sequenced.

Another aspect of the present disclosure relates to a method for next-generation sequencing of a DNA sample, comprising: obtaining a DNA sample from a biological source; fragmenting the DNA sample to obtain a plurality of DNA fragments; constructing a next-generation sequencing library of DNA fragments by a method described herein to generate a plurality of linearized double-stranded DNA molecules, wherein each strand comprises concatemer of top and bottom strands of a DNA fragment; and determining the sequence of the top and bottom strands of the DNA fragment using next-generation sequencing with read primers that bind to the linearized double-stranded DNA molecule, thereby obtaining the sequence of the DNA molecule.

In some embodiments, the biological sample is blood. In some embodiments, the biological sample is a sample of tissue from liver, kidney, brain, heart, skin, lung, colon, or pancreas. In some embodiments, the biological sample a sample of a diseased tissue from liver, kidney, brain, heart, skin, lung, colon, or pancreas. In some embodiments, the diseased tissue is a proliferative disease. In some embodiments, the diseased tissue is a tumor. In some embodiments, the sequencing error rate is similar to a control based on Duplex Sequencing, but wherein the number of reads required is decreased by at least 100-fold.

Another aspect of the present disclosure relates to the isolated nucleic acid complex as described herein for use in a method of methylation sequencing, wherein at least one oligonucleotide is modified to contain methylated cytosine in place of unmethylated cytosine.

Another aspect of the present disclosure relates to the isolated nucleic acid complex as described herein for use in a method of methylation sequencing, wherein each of the first, second, third, and fourth oligonucleotides is modified to contain methylated cytosine in place of unmethylated cytosine.

Another aspect of the present disclosure relates to the sequencing adapter as described herein for use in a method of methylation sequencing, wherein at least one oligonucleotide is modified to contain methylated cytosine in place of unmethylated cytosine.

Another aspect of the present disclosure relates to the sequencing adapter as described herein for use in a method of methylation sequencing, wherein each of the first, second, third, and fourth oligonucleotides is modified to contain methylated cytosine in place of unmethylated cytosine.

Another aspect of the present disclosure relates to a method of methylation sequencing of a DNA sample, comprising: ligating the first and second ends of a sequencing adapter described herein to a DNA fragment having opposing top and bottom strands, thereby forming a partially circularized DNA molecule comprising the DNA fragment and the sequencing adapter, wherein the sequencing adapter is modified to contain methylated cytosine in place of unmethylated cytosine; and synthesizing first and second single-strand DNA molecules by extending the free 3′ ends on the sequencing adapter each using the opposite strand of the partially circularized DNA molecule as a template, thereby forming a linearized double-stranded DNA molecule, wherein each strand comprises a concatemer of the top and bottom strands of the DNA fragment, wherein the synthesizing step comprises contacting the free 3′ ends with a DNA polymerase and methylated-dCTP along with standard dATP, dGTP and dTTP deoxynucleotides; deaminating unmethylated cytosines to uracils in the original top strand of the DNA fragment; determining the sequence of the top and bottom strands by next generation sequencing, comparing the sequences to infer methylation positions in the original DNA fragment. In some embodiments, the DNA sample is obtained from a biological sample. In some embodiments, the biological sample is obtained from liver, kidney, brain, heart, skin, lung, colon, or pancreas tissue, optionally wherein the tissue is diseased. In some embodiments, the disease is a proliferative disease. In some embodiments, the disease is a tumor.

In some embodiments, the dsDNA duplex is pre-amplified prior to the first step described above, the method comprising: contacting the dsDNA duplex with a first and a second pre-amplification molecule, wherein each of the two pre-amplification molecules comprises a UMI, a sample index, a rolling circle amplification (RCA) primer, and a truncation site; ligating the first pre-amplification molecule to one first end of the dsDNA duplex and ligating the second pre-amplification molecule to the second end of the dsDNA duplex to produce a pre-amplification dsDNA duplex; exposing the pre-amplification dsDNA duplex to a DNA polymerase enzyme; incubating the pre-amplification dsDNA duplex and the DNA polymerase enzyme for a sufficient time to complete RCA; and removing the RCA primer by cleaving the pre-amplification dsDNA duplex at the truncation site.

In some embodiments, the DNA duplexes to be sequences are pre-amplified prior to the first step described above, the method comprising: contacting each of the DNA duplexes to be sequenced with a first and a second pre-amplification molecule, wherein each of the two pre-amplification molecules comprises a UMI, a sample index, a rolling circle amplification (RCA) primer, and a truncation site; ligating the first pre-amplification molecule to one first end of each of the DNA duplexes to be sequenced and ligating the second pre-amplification molecule to the second end of each the DNA duplexes to be sequenced to produce a plurality of pre-amplification DNA duplexes; exposing each of the pre-amplification DNA duplexes to a DNA polymerase enzyme; incubating each of the pre-amplification DNA duplexes and the DNA polymerase enzyme for a sufficient time to complete RCA; and removing the RCA primer by cleaving each of the pre-amplification DNA duplexes at the truncation site.

Another aspect of the present disclosure relates to a method of preparing a next-generation sequencing library, comprising: blocking the 3′ end of R06 and the 3′ end of R05 from undergoing ligation; ligating the complex described herein to the dsDNA duplex as follows: ligating the 5′ end of R01 to the 3′ end of a first strand of the dsDNA duplex; and ligating the 5′ end of R10 to the 3′ end of a second strand of the dsDNA duplex; thereby forming a circular double-stranded DNA intermediate comprising the target DNA molecule and the complex; extending a first DNA strand from the 3′ end of R03; extending a second DNA strand from the 3′ end of R08; and circularizing each of the first and second DNA strands to form circular, single-stranded sequencing molecules; introducing a nick into a region between R03 and R08 to form linear, single-stranded sequencing molecules.

In some embodiments, the blocking of the first step described above comprises adding a blocking solution. In some embodiments, the ligating step of the second step described above comprises adding ligase. In some embodiments, the synthesizing of the third and fourth steps described above comprise contacting the circular double-stranded DNA intermediate with a polymerase. In some embodiments, the polymerase is a DNA-dependent DNA polymerase. In some embodiments, the polymerase has a strand-displacement activity. In some embodiments, the next-generation sequencing (NGS) is a short-read strategy.

In various embodiments, prior to CODEC library preparation and/or sequencing, the DNA fragments targeted for sequencing may be treated by conventional ER/AT repair. In other embodiments, prior to CODEC library preparation and/or sequencing, the DNA fragments targeted for sequencing may be treated by duplex repair.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1AR show an overview of Concatenating Original Duplex for Error Correction (CODEC) and validation of CODEC.

FIG. 1A shows standard NGS workflow (e.g., as with traditional duplex sequencing) involve dissociation of DNA duplex, which loses the intrinsic property of DNA that encodes genetic information twice. While both strands of a duplex can be tracked through unique molecular identifiers (UMIs) to identify false mutations caused by base damage, PCR, and NGS errors, finding them among billions of other strands costs throughput, highlighted by clusters. The CODEC workflow physically links each duplex before obtaining each sequencing read, ensuring each library molecule retains information of both strands for every sequence read since each sequence read will provide the concatenated top and bottom strand sequences for each DNA fragment in the library.

FIG. 1B shows CODEC links the sequence information of an original duplex into a single strand, i.e., each single strand sequence read will provide the concatenated top and bottom strand sequences for each DNA fragment in the library. As a result, each pair of NGS reads becomes self-sufficient for forming a duplex consensus (box). It utilizes the adapter complex instead of a duplex adapter for ligation, followed by strand displacing extension.

FIG. 1C shows CODEC modifies the adapter ligation step of ligation-based NGS workflows.

FIG. 1D shows CODEC adapter complex is prepackaged with all of the components needed for Illumina NGS (including read primer binding sites, flow cell binder region (i.e., the NGS adapters), UMI and indices regions, and dT-tails to facilitate ligation to DNA fragments). Unlike standard NGS libraries, CODEC reads outward to sequence a UMI, an index, and an insert together. No indexed primers are required as indices and flow cell binding regions (P5 and P7) are added by the ligation. As shown in FIG. 1B, the CODEC adapter (left construct) is ligated to each end of a DNA fragment (having a top and bottom strand), thereby producing a partially circularized, partially double-stranded DNA intermediate (see FIG. 1B) that includes the DNA fragment to be sequence joined at each end to the CODEC adapter. The partially circularized intermediate, partially-double stranded intermediate then undergoes strand displacing extension with a DNA polymerase which extends from the free 5′ ends of the central duplex region located in the adapter portion of the circularized intermediate. The DNA polymerase extends from each of the 5′ ends to synthesize single strand DNAs

FIG. 1E shows double-stranded regions of the adapter are predicted to stay stable with oligonucleotide concentrations of 500 nM at 20 C and Na at 10 mM. FIG. 1F shows the length of single-stranded linkers was determined to mitigate bending stiffness of a target duplex. FIG. 1G shows a CDS product contains two duplexes, one created from Stand 1 and another from Strand 2, with a linker in between and NGS adapters on both ends. FIG. 1H shows that CDS starts with a circular ligation between an insert and an adapter complex. The extension is then performed by a polymerase with strand displacement activity, starting from open 3′-ends. FIG. 11 shows CDS can be integrated into a conventional workflow of whole genome sequencing (WGS), whole exome sequencing (WES), or targeted sequencing by replacing an adapter ligation step. FIG. 1J shows that WGS with Illumina MiSeq (2×300 bp) confirmed that 56.7% of total reads had the correct structure and its consensus error rate was similar to the raw rate squared, as expected. FIG. 1K shows that with an additional CDS read primer that sequences Strand 2 in a synchronized manner, dual fluorescence is generated at each cycle during Read 1. Any disagreement between two strands will be marked by low Q-score. FIG. 1L is a schematic showing a variant Concatenated Duplex Sequencing (CDS). FIG. 1M is a schematic showing the integration of CDS with an Illumina workflow. FIG. 1N shows the long duplex with mismatch bubbles variant. FIG. 1O shows the modular duplex with mismatch bubbles variant. FIG. 1P shows the half adapter complex variant. FIG. 1Q shows the UMI variant. FIG. 1R shows the variant with regions 2 and 3 as partial read primer binding sites. FIG. 1S shows the variant with regions 2 and 3 as complete read primer binding sites. FIG. 1T is a schematic showing formation of a variant with region 1 as indices. FIG. 1U is a schematic showing the mechanism by which CDS adapter complex creates the concatenated structure. FIG. 1V is a schematic showing CDS. FIG. 1W is a schematic showing that CDS structure ignores single insert byproducts which impact NGS quality. FIG. 1X shows the mechanism of single-insert byproduct formation during bridge amplification, leading to mixed clusters, FIG. 1Y shows evidence of mixed cluster formation when NGS read primer binding sites are on the outer ends as in the simple concatenation approach drawn in FIG. 1W. Q scores are plotted versus position in read, where end of the insert is marked by vertical line, and bases shared in common between CDS linker sequence and SI adapter sequence are annotated with red dots. The higher base quality scores at shared bases indicates mixed fluorescence from CDS and SI byproducts. FIG. 1A shows the median Q-score in the region read after the insert for ‘simple concatenation’ vs. CDS as shown in FIG. 1W. With simple concatenation, the low median Q-score for bases which are unique vs. shared between the CDS linker and p7 adapter indicates that single-insert byproducts are being read too. Whereas, with CDS, the high median Q-scores in the region after the insert has been read indicates that single-insert byproducts are now being ‘ignored’ from the mixed clusters. FIG. 1AA is a schematic showing CDS attaches indices right next to inserts and earlier in sample preparation. FIG. 1AB is a schematic showing a CDS adapter complex for next-generation sequencing of a target double stranded DNA claims directed to novel CDS adapter complex FIG. 1AC is a schematic showing a duplex for sequencing a target double stranded DNA. FIG. 1AD is a schematic showing a method of forming a duplex for sequencing a target double stranded DNA. FIG. 1AE is a schematic showing a method of next-generation sequencing of a target double stranded DNA FIG. 1AF shows that CDS methods and compositions may be combined with Duplex-Repair. FIG. 1AG is a schematic showing duplex sequencing. Duplex sequencing can be 1000× more accurate than traditional sequencing and functions based on premise that true mutations will be on both strands of the same DNA duplex. It uses standard adapters with molecular barcodes that enable NGS reads to be linked back to each original DNA molecule, to form a ‘duplex consensus’. However, it requires about 100× more NGS reads to ‘find’ both strands of each duplex which is not possible for exomes/genomes; severely limiting for gene panels. FIGS. 1AH-1AJ show the CDS mechanism. FIG. 1AK shows that concatenated duplex sequencing (CDS) links both strands of each duplex such that they can be sequenced together within single read pairs. FIG. 1AL shows key steps involved in most NGS workflows. FIG. 1AM shows that Duplex-Repair limits strand resynthesis prior to adapter ligation, and thus, the potential for base damage errors to be copied both strands-which happens with commercial ER/AT methods. The length of dsDNA is shorter along its axis compared to when it is single-stranded. Duplexes with up to 174 bp can be accommodated without bending at all. FIG. 1AN shows an overview of Duplex-Repair, Duplex-Repair v2 vs. conventional ER/AT methods; FIG. 1AO shows a schematic of the major products of various synthetic duplexes subjected to each step of Duplex-Repair and conventional ER/AT as determined by capillary electrophoresis. The non-fluorophore-tagged ends of the synthetic molecules are depicted, and fragment sizes are drawn to scale. Duplexes demarcated by asterisks (*) do not contain fluorophores and were not directly observed by capillary electrophoresis; however, their presence is predicted due to the characterized activities of UDG and FPG. Regions of strand resynthesis are illustrated in light blue; FIG. 1AP shows the measured library conversion efficiencies of Duplex-Repair vs. the KAPA HyperPrep kit as a function of DNA input by using a ddPCR assay. FIG. 1AQ shows duplex pre-amplification creates multiple copies of each original duplex including strand identifiers, unique molecular identifiers (UMIs), and sample indices. Using endonuclease digestion, copies of each original duplex are released from each amplicon and are ready to be used in CODEC strand linking. FIG. 1AR shows that CODEC v2 now ligates adapter oligonucleotides separately and assembles the adapter complex afterwards. By using this new ligation strategy, the first two adapter oligonucleotides are ligated utilizing 3′-end blocked oligonucleotides to ligate only one strand of each duplex, followed by displacing the blocked oligonucleotides with remaining adapter oligonucleotides for the second ligation. Removing adapter blockers allow assembling the adapter complex, which can be used as a template for strand displacing extension.

FIG. 2 show the theory behind CODEC adapter complex design and read primer binding sites of standard NGS and CODEC.

FIGS. 3A-3B. FIG. 3A shows that during cluster generation cycles on an NGS flow cell, early termination in the middle of the insert region could create byproducts which turn into shorter fragments with only one insert and the read primer binding regions. These subclonal fragments have the same sequence as the correct fragments until the shared region ends. After sequencing cycles pass the shared region, the short fragments cause mixed fluorescence, and consequently, low Quality Scores. FIG. 3B shows Mean Quality Scores of each sequencing cycle by taking the last 150 bp in the shared region and the first 50 bp after the shared region from randomly selected 100 read pairs. Before redesigning the adapter structure, Quality Scores suddenly dropped after the shared region, making it difficult to confirm whether a read has the CODEC structure or not. This issue was solved by moving the read primer binding regions to the linker in order to ‘silence’ all byproducts without the linker.

FIG. 4 shows UMIs and each set of 4 indices are designed to collectively include all four bases at each position while keeping similar hybridization 6-G (FIG. 1AL) for high-quality image analysis of Illumina sequencers. For example, Illumina software uses up to first 25 bp for various purposes such as cluster identification, phasing correction, and chastity filter. Sequences shown from top to bottom correspond to SEQ ID NOs: 19-26.

FIGS. 5A-5B. FIG. 5A shows ratios of the correct CODEC product and byproducts which have been named after how they were likely created. FIG. 5B shows expected mechanisms of byproduct formation. “Double ligation” can occur when two adapter complexes are ligated to each end of an insert and go through T/T mismatched ligation with each other, as opposed to A/T ligation. “Blank ligation” can occur when two adapter complexes go through T/T mismatched ligation on both ends with each other with no insert. “Intermolecular” can occur when a strand displacing extension uses another ligation product as a template instead of the opposite strand linker.

FIGS. 6A-6B show proof-of-concept. FIG. 6A shows error rates of CODEC, Duplex Sequencing, and other consensus methods including typical paired-end read (R1+R2) and single strand consensus (SSC). Target enrichment with a pan-cancer gene panel was performed on cell-free DNA (cfDNA) of two individuals. Error bars indicate 95% binomial confidence intervals. FIG. 6B shows error rates at each family size, which is the number of raw reads with the same UMI and start-stop positions. FIG. 6C shows that with a pan-cancer panel, CDS showed comparable single nucleotide variant (SNV) error rates to Duplex Sequencing when applied to cell-free DNA (cfDNA) of two individuals, which were much lower than that of typical paired-end reads (R1+R2) or single strand consensus (SSC). Error bars indicate binomial confidence (95%) intervals. FIG. 6D shows that even with fewer raw reads, CDS had higher mean unique depth (3.96), whereas Duplex Sequencing had near-zero unique depth (0.025). Lines indicate cumulative fractions, FIG. 6E shows that the SNV error rate of CDS was still comparable to that of Duplex Sequencing. FIG. 6F shows that CDS showed superior precision than paired-end reads when the minimum allele threshold was 1, while maintaining the recall.

FIG. 7 is a schematic showing that deaminated cytosines, which are uracils, on overhangs of input samples went through end-repair and strand displacing extension. Phi29 DNA polymerase used for the extension can recognize uracils unlike HiFi polymerases and may have created a strand that can be amplified in a subsequent PCR (Crick strand). In case a sample had a high level of deamination, USER enzyme step was added to CODEC workflow in order to suppress false positives from uracils.

FIGS. 8A-8C show the characterization of CDS in targeted panel sequencing. FIG. 8A shows error rates for pan-cancer panel as a function of sequence context. C>T error rate of CODEC from a healthy donor was higher than that of Duplex Sequencing. FIG. 8B shows that in healthy donor cfDNA, CDS started to recover unique original duplexes 350 times faster than Duplex Sequencing in pan-cancer panel sequencing. Solid lines show moving averages and shades indicate standard deviations. FIG. 8C shows CODEC Error rates at each family size, which is the number of raw reads with the same UMI and start-stop positions. CDS needed only a single paired read (i.e., family size=1) to achieve low SNV error rate, while forming a consensus of multiple CDS reads from the same original DNA molecule (i.e., family size>1) had little impact on error rates.

FIG. 9A-9F show duplex consensus data compared to Duplex Sequencing. FIG. 9A shows that in duplex consensus data, higher mean error rates of 12 bp from both fragment ends than those of the middle regions imply base damage at 5⁰-overhangs before end-repair, which was previously observed in other studies using Duplex Sequencing. This is because end-repair fills in 5⁰-overhangs and copies damaged bases on one strand to both strands and creates false duplex consensuses. In contrast, SSC corrects base damage at neither overhangs nor duplex regions, and thus, shows less error rate differences between the last 12 bp and the middle regions. FIG. 9B shows that CDS links both strands within a single library molecule, such that both can be read with single read pair. FIG. 9C shows a comparison of error rates in standard NGS (consensus=none), single-strand consensus sequencing, and double-strand (or duplex) consensus sequencing, from 271 cell-free DNA samples. FIG. 9D shows error rates vs. number of reads per sequence. FIG. 9E shows simulated duplex recovery against read depth for duplex sequencing of 20 ng of DNA versus what is theoretically attainable if each read pair reflected a unique DNA duplex. FIG. 9F shows aggregate duplex error rates for 271 cfDNA samples vs. 2 formalin-fixed paraffin-embedded (FFPE) tumor biopsies.

FIGS. 10A-10L show efficacy of CODEC-based sequencing. FIG. 10A shows overall error rates and their base contexts of WES on a FFPE sample. FIGS. 10B-10I show that CDS can resolve inexplicable errors in Duplex-Repair. FIG. 10J shows Whole-Genome Sequencing (WGS) costs vs error rates in four sequencing technologies: CODEC, Duplex Sequencing, Standard NGS, and Pacbio HiFi. Pacbio HiFi's median accuracy was taken as Q30 (99.9%) based on the product brochure. The rest of the data were generated at Broad and sequenced costs were calculated based on Broad Genomic Platform prices on Illumina NovaSeq S4 and Pacbio Sequel IIe. The standard NGS accuracy was based on consensus accuracy of R1+R2 at minimum Q30 base quality FIG. 10K shows a cumulative distribution indicating the proportion of total bases that were covered for at least a given coverage in the WGS data. The CODEC and Standard WGS were matched at mean coverage at 12×. FIG. 10L show error rates of CODEC vs Standard NGS in Whole-Exome Sequencing (˜40 Mb) of a blood normal sample. Left side, overall error rate and right side, broken down by mononucleotide sequence context. Error bars indicate binomial confidence (95%) intervals.

FIGS. 11A-11C show whole-genome sequencing (WGS) of the pilot genome NA12878 of the Genome in a Bottle Consortium. FIG. 11A shows error rates and sequencing costs of different methods. PacBio HiFi data used technical specification. FIG. 11B shows fractions of each unique duplex depth of CODEC and Duplex Sequencing. FIG. 11C shows false positives and false negatives of CODEC and R1+R2 when downsampled to lower depths.

FIGS. 12A-12E show data from use of CODEC for sequencing patient data. FIGS. 12A-12B shows overall error rates and their base contexts of WGS on an NA12878 sample. FIG. 12C shows that reading the same strand twice with paired-end reads (R1+R2) improved the error rate only by 4-fold, whereas reading both the original top and bottom strands (duplex sequencing and CDS) improved it by 1100-fold. Error bars indicate binomial confidence (95%) intervals. FIG. 12D shows that CDS recovered original duplexes more efficiently with less reads than duplex sequencing, and its lower plateau implies further optimization is needed for CDS+hybrid capture enrichment workflow. Dotted lines indicate simulated curves shown in FIG. 9E. FIG. 12E shows that when applied to a patient-specific assay targeting 76 sites as per Parsons et al., CDS successfully detected somatic mutations from a cancer patient's cfDNA with perfect specificity. Horizontal lines, boxes, and whiskers indicate medians, 25-75% ranges, and 5%-95% ranges, respectively.

FIGS. 13A-13C show indels at Mononucleotide microsatellites. FIG. 13A shows Summarized indel error frequencies at mononucleotide microsatellites of NA12878. FIG. 13B shows Indel error frequencies at mononucleotide microsatellites with different lengths from 8 to 18 nucleotides. FIG. 13C shows Microsatellite instability (MSI) detection limit. Tumor and normal samples of a colon cancer patient with MSI were sequenced and diluted in silico.

FIGS. 14A-14I show trinucleotide contexts and Catalogue Of Somatic Mutations In Cancer (COSMIC) signatures from WGS on the MSI sample. FIG. 14A shows standard NGS can only detect high-abundance mutations from multiple molecules but not low-abundance mutations which are obscured by background noise. CODEC can call both high- and low-abundance mutations due to its single duplex resolution. FIG. 14B shows mutational contexts after thresholding mutations with or without a variant caller, Mutect2. Selecting only high-abundance mutations has been the gold standard for standard NGS. Each bar represents a trinucleotide context. FIG. 14C shows cosine similarities against high-abundance mutations selected by Mutect2 from standard NGS at 12× coverage (dashed box). Each method was downsampled to lower depths until observing a significant drop in its cosine similarity. FIG. 14D shows rate of mutations detected by CODEC but not selected by Mutect2. FIG. 14E shows COSMIC single base substitution (SBS) signatures extracted from different groups of mutations. Groups under ‘Not called by Mutect2’ are subsets of corresponding groups under ‘All mutations’. FIG. 14F shows the previously described workflow for qualification and sequencing of tumor whole-exomes from plasma cfDNA (Adalsteinsson et al. Nat Comms 2017), FIG. 14G shows the estimated fractions of tumor-derived cfDNA in 520 patients with Stage IV breast and prostate cancer, showing that only 33-45% have sufficient tumor content for plasma whole-exome sequencing, FIG. 14H shows the overlap in clonal and subclonal tumor mutations between whole-exome sequencing of cfDNA and matched tumor biopsies from patients with >0.1 tumor fraction in cfDNA, and FIG. 141 shows a demonstration of serial whole-exome sequencing being used to monitor cancer progression and evolution in a patient with metastatic breast cancer, identifying the emergence of what could be convergent evolution of drug resistance (e.g. multiple ESR1 mutations) in response to treatment with a selective estrogen receptor degrader.

FIG. 15 shows a binomial model comparing abilities of detecting low-abundance mutations (low variant allele fraction) between CODEC and standard WGS at different coverages (30×, 60×, and 80×). Standard WGS required at least two unique fragments for error correction. Thus, this model ignored sequencing errors. Below 0.3% VAF, CODEC showed better detection power than 30× standard WGS. Below 0.03% VAF, CODEC showed superior sensitive than any of the higher depth standard WGS.

FIG. 16 is a schematic showing the protocol developed to enable CDS to retain and report DNA methylation information.

FIGS. 17A-17D show quantification of strand resynthesis during ER/AT using the Kapa HyperPrep kit. FIG. 17A shows a schematic of a method for quantifying fill-in bases during ER/AT. FIG. 17B shows measured interpulse duration (IPD; in frames) as a function of the base position on five synthetic oligonucleotides. Longer IPDs, gray if greater than 60 frames, result from modified bases. Dashed lines indicate where fill-in is expected to start during ER/AT. FIG. 17C shows measured IPD as a function of the base position on a healthy donor cfDNA sample. FIG. 17D shows four highlighted duplexes that underwent extensive strand resynthesis.

FIGS. 18A-18I show a comparison of Duplex Repair to conventional ER/AT. FIG. 18A shows the performance of the Duplex-Repair approach, in comparison to conventional ER/AT, on multiple different synthetic oligonucleotides as determined by capillary electrophoresis (i-vii). FIG. 18B shows measured duplex sequencing error rates using Duplex-Repair vs. commercial ER/AT and the IDT xGEN ‘pan-cancer’ panel applied to healthy donor cfDNA treated with varied amounts of DNase I (to induce nicks) and CuCl2/H2O2 (to induce oxidative damage). FIG. 18C shows duplex sequencing error rates after using Duplex-Repair vs. conventional ER/AT to repair formalin fixed tumor DNA. (Of note, the wider error bars for Duplex-Repair samples were due to fewer total duplexes sequenced). FIG. 18D shows estimated fractions of interior base pairs (>12 bp from either end of the original duplex fragment) that were resynthesized using conventional ER/AT and several variations of Duplex-Repair, as measured using a custom single-molecule sequencing assay. FIG. 18E shows the estimated fraction of interior base pairs resynthesized for both conventional ER/AT and Duplex-Repair across three sample types. FIG. 18F shows duplex sequencing error rates of four healthy cfDNA samples (three replicates per condition), three cancer patient cfDNA samples (one replicate per condition), and five cancer patient FFPE tumor biopsies (three replicates per condition) treated with conventional ER/AT or Duplex-Repair. FIG. 18G shows the aggregate mutant bases and their position relative to the end of the original duplex fragment. Dashed line represents the threshold of the interior of the fragment (12 bp). FIG. 18H shows measured duplex sequencing error rates of HD_78 cfDNA damaged with varied concentrations of DNase I (to induce nicks) and CuCl2/H₂O₂ (to induce oxidative damage) and then repaired by using Duplex-Repair or conventional ER/AT (three replicates per condition). FIG. 18I shows a comparison of conventional ER/AT and Duplex-Repair for cfDNA and FFPE sample types shows comparable duplex recoveries as a function of the number of read pairs, as analyzed via in silico downsampling of reads.

FIGS. 19A-19B show non-limiting problems that may be addressed by CODEC sequencing CODEC sequencing. FIG. 19A shows that sequencing has gotten cheaper but not more accurate. This has severe implications for all types of DNA sequencing in biomedical research and diagnostics. FIG. 19B shows the potential of CDS to ‘clean up’ all types of DNA sequencing.

FIG. 20 illustrates that duplex pre-amplification may be conducted on a nucleic acid sample (e.g., a DNA sample) prior to CODEC adapter ligation and CODEC sequencing.

FIG. 21 illustrates an embodiment of CODEC sequencing using a modified CODEC sequencing adapter.

### DETAILED DESCRIPTION

The present disclosure provides a novel DNA sequencing method referred to herein as “Concatenating Original Duplex for Error Correction” or “CODEC” that improves upon duplex sequencing, as well as to compositions for conducting said novel sequencing method (e.g., a multi-oligonucleotide adapter for library production, adapter constructs, and sequencing libraries), methods for making the adapters, methods for library construction, and duplex sequencing methods that improve the accuracy of duplex sequencing and at a lower cost. In various aspects, library preparation using CODEC adapters results in each DNA molecule becoming self-sufficient for forming a duplex consensus, facilitating the identification of true mutations and avoiding false mutations.

In various aspects, the disclosure provides a powerful new library construction method that concatenates both strands of each DNA duplex into a linear sequence. By physically linking both strands, the products are self-sufficient to form a duplex consensus. This strategy has the potential to provide 1,000-fold more accurate sequencing with minimal added cost, and could directly enhance existing products (WGS, WES, targeted panels) offered at the Genomics Platform.

In various aspects, the disclosure provides methods for CODEC sequencing as well as compositions required for and/or produced by CODEC sequencing, including adapters (referred to herein in various embodiments as “CODEC adapters”), circularized intermediates each comprising a CODEC adapter ligated to both ends of a DNA fragment to be sequenced (referred to herein in various embodiments as “CODEC circularized intermediates”), and linearized double-stranded products comprising concatenated top and bottom strands of the single DNA fragments to be sequenced (referred to herein in various embodiments as “the CODEC library” or individually as “CODEC library members”). In various embodiments, the CODEC adapter includes NGS adapters for NGS workflow (e.g., cluster amplification on NGS flow cell), sequencing read primer sites for reading both strands of a DNA fragment, and optionally one or more sample indices and one or more unique molecular identifiers (UMIs).

Unlike traditional duplex sequencing which separately obtains the top and bottom strand sequences for each DNA fragment to be sequenced (and thus, requiring computational approaches to identify, match, and compare the top and bottom strand sequences), each of the CODEC library members is self-sufficient for forming a duplex consensus sequence in the same read because library formation using the CODEC adapter results in double-stranded library members whereby each strand comprises a concatemer of top and bottom sequences of each original DNA fragment (i.e., in the same DNA molecule) to be sequenced. Thus, sequencing of the CODEC adapter results in a sequencing product that comprises the top strand, the bottom strand, and optionally one or more sample indices and one or more UMIs. The technical advantage of this approach, as compared to standard duplex sequencing, is that unlike standard duplex sequencing which generates two separate sequencing products (i.e., one for the top sequence and one for the bottom sequence), CODEC sequencing results in a single sequencing product that comprises both the top sequence and the bottom sequence thereby allowing a user to easily discern true mutations (mutations that appear in both the top and bottom portions of the sequencing read) from false mutations (mutations that appear in only the top or bottom portion of the sequencing read).

Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 3D ED., John Wiley and Sons, New York (2006), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference. The meaning and scope of the terms are clear; however, in the event of any latent ambiguity, definitions provided herein take precedent over any dictionary or extrinsic definition. Further, unless otherwise required by context, singular terms shall include pluralities and plural terms shall include the singular. In this disclosure, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics, and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present disclosure unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of subjects.

The terms “approximately” or “about,” as may be used interchangeably herein, and as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction of (i.e., percentage greater than or percentage less than) the stated reference value unless otherwise stated or otherwise evident from the context (for example, when such number would exceed 100% of a possible value).

The term “dA-tailing,” as may be used herein, refer to the status, or to a characteristic, of a nucleic acid (e.g., DNA, RNA) as having a “tail” comprising a non-templated adenosine (A) (e.g., adenosine monophosphates). By “tail” it is meant that the adenosines (e.g., AAAAA) at the 3′ end of the nucleic acid (e.g., DNA, RNA), comprises an overhang beyond the 5′ terminal nucleotide of the complementary strand. The term (e.g., dA-tail) may be used as a verb (e.g., dA-tailing) to describe the process by which the adenosine is added to the 3′ end of a nucleic acid. In some embodiments, dA-tailing is performed using Taq polymerase. In some embodiments, dA-tailing is performed using Klenow Fragment lacking 3′ to 5′ exonuclease activity.

The term “overhang,” as may be used herein, is a term of art known to the skilled artisan to refer to a portion of a double-stranded nucleic acid which extends (e.g., protrudes) beyond the end (e.g., terminal nucleotide) of the opposing strand (e.g., complementary strand). For example, without limitation, a 5′ overhang will refer to the portion of a strand of a nucleic acid which extends beyond the 3′ end (3′ terminal nucleotide) of the opposing strand (e.g., complementary strand) with which it forms a double-stranded nucleic acid duplex. As an additional example, without limitation, a 3′ overhang will refer to the portion of a strand of a nucleic acid which extends beyond the 5′ end (5′ terminal nucleotide) of the opposing strand (e.g., complementary strand) with which it forms a double-stranded nucleic acid duplex. As will be appreciated by the skilled artisan, a double-stranded duplex, may comprise both a 5′ and 3′ overhang, a single 5′ overhang, two 5′ overhangs, a single 3′ overhang, two 3′ overhangs, an overhang (e.g., 5′ or 3′) and a blunt end, or two blunt ends. As used herein, the term “blunt end,” refers the quality of double-stranded duplex, wherein the two strands forming the duplex terminate at the same pair of nucleotides and thus has no overhang at that end of the duplex (e.g., the end is blunt).

The term “exonuclease,” as may be used herein, refers to the term of art generally known to the skilled artisan to refer to an enzyme that has at least the activity of cleaving nucleotides from the end of a nucleic acid (e.g., polynucleotide, oligonucleotide). In some embodiments, an exonuclease will cleave the nucleotides one at a time. An exonuclease may cleave nucleotides in either direction (e.g., from either the 5′ or 3′ end) of a nucleic acid. When describing such activity, often the notation is shown to be 5′ to 3′ exonuclease activity, when referring to an exonuclease that cleaves nucleotides starting from the 5′ end of a nucleic acid (e.g., the 5′ nucleotide which is distal to the 3′ end) or 3′ to 5′ exonuclease activity, when referring to an exonuclease that cleaves nucleotides starting from the 3′ end of a nucleic acid (e.g., the 3′ nucleotide which is distal to the 5′ end). In some embodiments, an exonuclease has 5′ to 3′ exonuclease activity. In some embodiments, the exonuclease can be Exo VII.

The terms “complementary” and “complementarity,” as may be used interchangeably herein, refer a property of a nucleotide (e.g., A, C, G, T, U) in a nucleic acid (e.g., RNA, DNA) in a strand (e.g., oligonucleotide) to pair with another particular nucleotide in a nucleic acid strand of the opposite orientation (e.g., strands running parallel, but in the reverse direction (i.e., 5′-3′ aligns with 3′-5′, and 3′-5′ with 5′-3′)) (i.e., Watson-Crick base-pairing rules). With respect to deoxyribonucleic acids (DNA) the base pairings which are complementary are adenine (A) and thymine (T) (e.g., A with T, T with A) and guanine (G) and Cytosine (C) (e.g., G with C, C with G) and with respect to ribonucleic acid (RNA) the base pairings which are complementary are A and uracil (U) (e.g., A with U, U with A) and G and C (e.g., G with C, C with G). This occurs because of the ability of each base pair to form an equivalent number of hydrogen bonds with its complementary base (e.g., A-T/U, T/U-A, C-G, G-C), for example the bond between guanine and cytosine shares three hydrogen bonds compared to the A-T/U bond which always shares two hydrogen bonds.

When every base in at least one strand of a pair of nucleic acids is found opposite its complementary base pair, such strand is considered fully complementary to its sequence in the other strand. When one, or more, bases of such a strand is found in a position where it is opposite any other base excepting its complementary base pair, that base is considered “mis-matched” and the strand is considered partially complementary. Accordingly, strands can be varying degrees of partially complementary, until no bases align, at which point they are non-complementary.

### CODEC Adapters, Library Preparation, and Sequencing

In various aspects, the disclosure provides methods for CODEC sequencing as well as compositions required for and/or produced by CODEC sequencing, including adapters (referred to herein in various embodiments as “CODEC adapters”), circularized intermediates each comprising a CODEC adapter ligated to both ends of a DNA fragment to be sequenced (referred to herein in various embodiments as “CODEC circularized intermediates”), and linearized double-stranded products comprising concatenated top and bottom strands of the single DNA fragments to be sequenced (referred to herein in various embodiments as “the CODEC library” or individually as “CODEC library members”). In various embodiments, the CODEC adapter includes NGS adapters for NGS workflow (e.g., cluster amplification on NGS flow cell), sequencing read primer sites for reading both strands of a DNA fragment, and optionally one or more sample indices and one or more unique molecular identifiers (UMIs).

In some embodiments, a CODEC adapter complex consists of four hybridized oligonucleotides, which include every element required for both concatenation and adapter attachment. In some embodiments, the CODEC adapter complex comprises at least ten regions (R01-R10) in the following configuration:

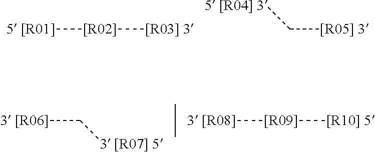

In some embodiments, [symbol] represents bonding. In some embodiments, R01, R02, and R03 comprise the first oligonucleotide, R04 and R05 comprise the second oligonucleotide, R06 and R07 comprise the third oligonucleotide, R08, R09, R10 comprise the fourth oligonucleotide. In some embodiments, R01 and R06 are annealed to one another, R03 and R08 are annealed to one another, R05 and R10 are annealed to one another, R02 and R07 are not annealed to one another, and R04 and R09 are not annealed to one another.

In some embodiments, a CODEC adapter complex is ligated (adapter ligation) with one end of a target duplex (target DNA molecule), followed by ligation between the other ends to produce circularized product. The term “adapter ligation,” as may be used herein, refers to the term as known to the skilled artisan to generally refer to the process of attaching (e.g., ligating) known sequences of nucleotides (e.g., nucleic acids, oligonucleotides, e.g., adapters) to one or more ends of one or more nucleic acids (e.g., DNA fragments, complementary strands of DNA). Often adapters contain specific sequences which are complementary to the nucleic acid fragments they are intended to attach to, for example, without limitation in the event nucleic acids are dA-tailed, an adapter may have a “T” overhang, wherein the “T” refers to a nucleotide comprising a thymine nucleobase. The T overhang is complementary to the dA-tail, thus facilitating ligation. The terms “complementary” and “complementarity,” as may be used interchangeably herein, refer a property of a nucleotide (e.g., A, C, G, T, U) in a nucleic acid (e.g., RNA, DNA) in a strand (e.g., oligonucleotide) to pair with another particular nucleotide in a nucleic acid strand of the opposite orientation (e.g., strands running parallel, but in the reverse direction (i.e., 5′-3′ aligns with 3′-5′, and 3′-5′ with 5′-3′)) (i.e., Watson-Crick base-pairing rules). With respect to deoxyribonucleic acids (DNA) the base pairings which are complementary are adenine (A) and thymine (T) (e.g., A with T, T with A) and guanine (G) and Cytosine (C) (e.g., G with C, C with G) and with respect to ribonucleic acid (RNA) the base pairings which are complementary are A and uracil (U) (e.g., A with U, U with A) and G and C (e.g., G with C, C with G). This occurs because of the ability of each base pair to form an equivalent number of hydrogen bonds with its complementary base (e.g., A-T/U, T/U-A, C-G, G-C), for example the bond between guanine and cytosine shares three hydrogen bonds compared to the A-T/U bond which always shares two hydrogen bonds. When every base in at least one strand of a pair of nucleic acids is found opposite its complementary base pair, such strand is considered fully complementary to its sequence in the other strand. When one, or more, bases of such a strand is found in a position where it is opposite any other base excepting its complementary base pair, that base is considered “mis-matched” and the strand is considered partially complementary. Accordingly, strands can be varying degrees of partially complementary, until no bases align, at which point they are non-complementary. Other non-standard nucleotides (e.g., 5-methylcytosine, 5-hydroxymethylcytosine) are known in the art and their properties and complementarity will be readily apparent to the skilled artisan.

In some embodiments, R01 comprises a first concatenated duplex sequencing (CDS) adapter; R02 comprises a single-stranded linker, first unique molecular identifier (UMI), and a first read primer site; R03 comprises a first sequence at or near the 3′ end capable of priming DNA synthesis by a DNA-dependent DNA polymerase; R04 comprises a free 5′ end comprising a first next-generation sequencing (NGS) adapter sequence; R05 comprises a third CDS adapter and a first sample index; R06 comprises a second CDS adapter and a second sample index; R07 comprises a free 5′ end comprising a second next-generation sequencing (NGS) adapter sequence; R08 comprises a second sequence at or near the 3′ end capable of priming DNA synthesis by a DNA-dependent DNA polymerase; R09 comprises a single-stranded linker, a second UMI, and a second read primer site; and/or R10 comprises a fourth CDS adapter.

The term “polymerase,” as may be used herein, is a term of art known to the skilled artisan to refer generally to an enzyme which aids in, or synthesizes nucleic acids (e.g., DNA polymerase, RNA polymerase) and polymers. There are known a multitude of polymerases, for example, without limitation and which are all contemplated herein, DNA polymerase I (Pol gamma, Pol theta, Pol nu), DNA polymerase II (Pol alpha, Pol delta, Pol epsilon, Pol zeta), DNA polymerase III holoenzyme, DNA polymerase IV (DinB) (SOS repair polymerase, Pol beta, Pol lambda, Pol mu), DNA polymerase V (SOS polymerase, Pol eta, Pol iota, Pol kappa), Reverse transcriptase, and RNA polymerase (RNA Pol I, RNA Pol II, RNA Pol III, T7 RNA Pol, RNA replicase, Primase). Additionally, as is further contemplated, are polymerases from bacterium (e.g., Thermus aquaticus). For example, Taq from Thermus aquaticus is a common DNA polymerase used in polymerase chain reactions (PCR). In some embodiments, a polymerase is a Taq polymerase. In some embodiments, a polymerase lacks 3′ to 5′ exonuclease activity. In some embodiments, a polymerase is a Klenow fragment. In some embodiments, a polymerase is a Klenow fragment lacking 3′ to 5′ exonuclease activity. In some embodiments, a polymerase is a human variant of any of the polymerases described herein.

In various embodiments, exemplary CODEC adapter oligonucleotide sequences are provided in Table 2 of Example 1.

The term “unique molecular identifier (UMI),” refers to a short oligonucleotide molecular barcode that provides error correction and increased accuracy during sequencing.

The terms “nucleic acid,” “nucleotide sequence,” “polynucleotide,” “oligonucleotide,” and “polymer of nucleotides,” as may be used interchangeably herein, refer to a string of at least two, nucleobase-sugar-phosphate combinations (e.g., nucleotides) and includes, among others, single stranded and double stranded DNA, DNA that is a mixture of single stranded and double stranded regions, single stranded and double stranded RNA, and RNA that is mixture of single stranded and double stranded regions, hybrid molecules comprising DNA and RNA that may be single stranded or, more typically, double stranded or a mixture of single stranded and double stranded regions. In addition, the terms (e.g., nucleic acid, et al.) as used herein can refer to triple stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple helical region often referred to as an oligonucleotide.

The terms (e.g., nucleic acid, et al.) also encompass such chemically, enzymatically, or metabolically modified forms of nucleic acids, as well as the chemical forms of DNA and RNA characteristic of viruses and cells, including simple and complex cells. For instance, the terms (e.g., nucleic acid, et al.) as used herein can include DNA or RNA as described herein that contain one or more modified bases. The nucleic acids may also include natural nucleosides (i.e., adenosine, thymidine, guanosine, cytidine, uridine, deoxyadenosine, deoxythymidine, deoxyguanosine, and deoxycytidine), nucleoside analogs (e.g., 2 aminoadenosine, 2 thiothymidine, inosine, pyrrolo pyrimidine, 3 methyl adenosine, 5 methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, 0(6) methylguanine, 4 acetylcytidine, 5 (carboxyhydroxymethyl)uridine, dihydrouridine, methylpseudouridine, 1 methyl adenosine, 1 methyl guanosine, N6 methyl adenosine, and 2 thiocytidine), chemically modified bases, biologically modified bases (e.g., methylated bases), intercalated bases, modified sugars (e.g., 2′ fluororibose, ribose, 2′ deoxyribose, 2′ O methylcytidine, arabinose, and hexose), or modified phosphate groups (e.g., phosphorothioates and 5′ N phosphoramidite linkages). Thus, DNA or RNA including unusual bases, such as inosine, or modified bases, such as tritylated bases, to name just two examples, are nucleic acids as the term is used herein. The terms (e.g., nucleic acid, et al.) also includes peptide nucleic acids (PNAs), phosphorothioates, and other variants of the phosphate backbone of native nucleic acids. Natural nucleic acids have a phosphate backbone, artificial nucleic acids can contain other types of backbones, but contain the same bases. Thus, DNA or RNA with backbones modified for stability or for other reasons are nucleic acids as that term is intended herein.

The term “nucleobase,” as may be used herein, is a term of art known to the skilled artisan as a nitrogenous base, which is a nitrogen-containing biological compound that forms a component of a nucleoside, which is itself a component of a nucleotide. The nucleobases (also referred to herein as simply a base), are one of the basic building blocks of nucleic acids (e.g., DNA, RNA) as they possess the ability to form base pairs and to stack one upon another and forming the long-chain helical structures. There are five canonical nucleobases: adenine (A), cytosine (C), guanine (G), thymine (T), and uracil (U), with A, C, G, and T being found in DNA and A, C, G, and U being found in RNA.

The term “nucleoside,” as may be used herein, refers to glycosylamines (e.g., N-glycosides) that are generally known to be nucleotides without a phosphate group. A nucleoside consists of a nucleobase (e.g., a nitrogenous base) and a five-carbon sugar (e.g., pentose). The five-carbon sugar can be either ribose or deoxyribose. Nucleosides are the biochemical precursors of nucleotides, which are the constituent components of RNA and DNA. Examples of nucleosides include cytidine (C), uridine (U), adenosine (A), guanosine (G), thymidine (T), and inosine (I), but includes variants (e.g., modified or synthetic nucleosides, nucleosides containing modified or synthetic nucleobases).

The term “nucleotide,” as may be used herein is a term of art known to the skilled artisan to generally refer to those compositions comprising a nucleobase, sugar, and phosphate (e.g., a nucleoside and a phosphate) (which compositions (e.g., nucleotides) are separated into purines and pyrimidines). Nucleotides are components of nucleic acids that can be copied using a polymerase. Nucleosides, cytidine (C), uridine (U), adenosine (A), guanosine (G), thymidine (T), and inosine (I), along with a phosphate group, represent the canonical nucleotides, and may be referred to in DNA form (e.g., with a deoxyribose) as dATP, dGTP, dCTP, and dTTP when referring to individual nucleotides used in a synthesis reaction (e.g., nucleotide with 3 phosphate groups (e.g., “tri-phosphate”)). Two of the phosophate groups may be hydrolyzed to yield a monophosphate nucleotide for use in the polymerization of a nucleic acid. Generally, dATP, dGTP, dCTP, and dTTP may be referred to as dNTPs, wherein “N” represents the ambiguity as to the nature of the nucleoside. Thus, a mixture of dNTPs may include a concentration of all or some of each. Nucleotides contain not only the known purine and pyrimidine bases, but also other heterocyclic bases that have been damaged (e.g., bases that have oxidized, methylated, acylated, deadenylated, etc.). The term is well-known in the art and will be readily appreciated by the skilled artisan.

In various embodiments, the four CODEC adapter oligonucleotides may be annealed before (i.e., pre-annealed) ligation with DNA fragments to be sequenced. In various other embodiments, the four CODEC adapter oligonucleotides may be annealed during or contemporaneous to the ligation step.

The advantage of pre-annealing four oligonucleotides before ligation is that both ends always get different adapters, whereas ligation without hybridization results in 50% of the target ligating to the same adapter on both sides, which cannot be circularized. In some embodiments, a single A/T overhang is added at ligation sites to improve the yield. In some embodiments, DNA blunt ends or DNA sticky ends are added. In some embodiments, single-stranded DNA regions are incorporated into the CODEC complex to add flexibility for circularization.

In some embodiments, the first sequence and second sequence, further comprise the same or different primer binding sites. In some embodiments, the first and second primer sites are oriented to initiate sequencing by addition in opposing directions. In some embodiments, the first and second UMI are distinct.

In some embodiments, R01 comprises between 12 and 30 nucleotides, R02 comprises between 14 and 75 nucleotides, R03 comprises between 12 and 99 nucleotides, R04 comprises between 20 and 49 nucleotides, R05 comprises between 12 and 30 nucleotides, R06 comprises between 12 and 30 nucleotides, R07 comprises between 20 and 49 nucleotides, R08 comprises between 12 and 99 nucleotides, R09 comprises between 14 and 75 nucleotides, and/or R10 comprises between 12 and 30 nucleotides.

In some embodiments, R01 and R06 comprise a hybridization free energy of about −10 kcal/mol, about −15 kcal/mol, about −20 kcal/mol, about −25 kcal/mol, about −30 kcal/mol, or about −35 kcal/mol; R03 and R08 comprise a hybridization free energy of about −10 kcal/mol, about −15 kcal/mol, about −20 kcal/mol, about −25 kcal/mol, about −30 kcal/mol, about −35 kcal/mol, about −40 kcal/mol, about −45 kcal/mol, about −50 kcal/mol, about −55 kcal/mol, about −60; and/or R05 and R10 comprise a hybridization free energy of about −10 kcal/mol, about −15 kcal/mol, about −20 kcal/mol, about −25 kcal/mol, about −30 kcal/mol, or about −35 kcal/mol.

In some embodiments, R01 and R06 each comprise the same number of nucleotides, optionally wherein R06 has a one nucleotide overhang to facilitate ligation; R03 and R08 each comprise the same number of nucleotides; and/or R05 and R10 each comprise the same number of nucleotides, optionally wherein R05 has a one nucleotide overhang to facilitate ligation.

In some embodiments, R01 and R06 comprise sequences with at least 90% complementarity; R03 and R08 comprise sequences with at least 90% complementarity; and/or R05 and R10 comprise sequences with at least 90% complementarity.

In some embodiments, each R01, R06, R05, and R10 comprise the same number of nucleotides, optionally wherein R06 and R05 each have a one nucleotide overhang to facilitate ligation.

In some embodiments, R01 comprises a first concatenated duplex sequencing (CDS) adapter; R02 comprises a single-stranded linker; R03 comprises a 3′ end capable of priming DNA synthesis by a DNA-dependent DNA polymerase; R04 comprises a first UMI; R05 comprises a third CDS adapter; R06 comprises a second CDS adapter; R07 comprises a second UMI; R08 comprises a 3′ end capable of priming DNA synthesis by a DNA-dependent DNA polymerase; R09 comprises a single-stranded linker; and R10 comprises a fourth CDS adapter.

In some embodiments, the 5′ end of R01 is ligated to the 3′ end of a first strand of a target DNA duplex; the 3′ end of R05 is ligated to the 5′ end of the first strand of the target DNA duplex; the 5′ end of R10 is ligated to the 3′ end of a second strand of the target DNA duplex; the 3′ end of R06 is ligated to the 5′ end of the second strand of the target DNA duplex; forming a circularized DNA duplex or optionally a partially double-stranded circular DNA.

In some embodiments, the CODEC adapter complex may be prepared for NGS and used for a research or clinical purpose (e.g., identification of a mutation in a subject, diagnosis of a disease). The term “subject,” as used herein, refers to any organism in need of treatment or diagnosis using the subject matter herein. For example, without limitation, subjects may include mammals and non-mammals. In some embodiments, a subject is mammalian. In some embodiments, a subject is non-mammalian. As used herein, a “mammal,” refers to any animal constituting the class Mammalia (e.g., a human, mouse, rat, cat, dog, sheep, rabbit, horse, cow, goat, pig, guinea pig, hamster, chicken, turkey, or a non-human primate (e.g., Marmoset, Macaque)). In some embodiments, a mammal is a human.

The term “mutation,” as may be used herein, refers to a change, alteration, or modification to a nucleotide in a nucleic acid as compared to its wild-type sequence. For example, without limitation, mutations may include substitutions, insertions, deletions, or any combination of the same. In some embodiments, there at least one mutation. In some embodiments, there are more than one mutation. In some embodiments, where there is more than one mutation, the mutations are distinct (e.g., not of the same type (e.g., substitutions, insertions, deletions)). In some embodiments, where there is more than one mutation, the mutations are the same (e.g., not of the same type (e.g., substitutions, insertions, deletions)). Additionally, in some embodiments, the mutations result in a frameshift.

Mutations, which as described hereinabove, are regions (e.g., sections, portions, nucleobases, nucleosides, nucleotides) of a given nucleic acid (e.g., DNA, RNA) which differ as compared to their wild-type nucleic acid, will most often be reflected in each strand of a nucleic acid. That is to say that, when a mutation is present in a sample it and its complement will be observed in each strand of the nucleic acid when sequenced. This presents a problem however, when considering that a sample may contain single-stranded portions (e.g., gaps, overhangs), or areas which may instigate strand resynthesis (e.g., nicks). This problem presents because if a damaged base is present in such single-stranded region, or other region which is resynthesized, a damaged base may instruct the synthesis of its complementary strand to include a base which was not originally present in the nucleic acid from which the sample was generated (because damaged bases can affect non-canonical base pairings). The same could happen if one strand contains mismatched bases. In such instances, the mismatch will show a paired match in the re-synthesized complement instead of its native mismatched base. When this happens, a sequencing of both strands will read a mutation in each of the strands, thus show a mutation, however, this mutation may not be a true reflection of the original nucleic acid. Such mutations are termed “false mutations,” herein. False mutations are mutations which result from the resynthesis of complementary strands of nucleic acid, which do not represent the original (e.g., native, wild-type) complementary strand of nucleic acid from which the sample was obtained.

In some embodiments, the method or preparation of the CODEC adapter complex may be a method of preparing a double-stranded DNA molecule (dsDNA duplex) for use in next-generation sequencing (NGS) of a target DNA molecule, comprising ligating the complex of any one of claims 1-21 to the dsDNA duplex as follows: ligating the 5′ end of R01 to the 3′ end of a first strand of the dsDNA duplex; ligating the 3′ end of R05 to the 5′ end of the first strand of the dsDNA duplex; ligating the 5′ end of R10 to the 3′ end of a second strand of the dsDNA duplex; and ligating the 3′ end of R06 to the 5′ end of the second strand of the dsDNA duplex; thereby forming a circular double-stranded DNA intermediate comprising the target DNA molecule and the complex; extending a first DNA strand from the 3′ end of R03; extending a second DNA strand from the 3′ end of R08; and optionally annealing the first and second DNA strands to form a double-stranded DNA molecule for use in next-generation sequencing (NGS) of the target DNA molecule. In some embodiments, the double-stranded DNA molecule comprises two copies of the target DNA molecule. In some embodiments, the ligating step comprises adding ligase. In some embodiments, the synthesizing steps comprise contacting the circular double-stranded DNA intermediate with a polymerase. The term “contacted,” as may be used herein, is used to describe the exposure of one substance (e.g., enzyme, reagent, dNTP) to another substance (e.g., sample, mixture), in an amount and with the intention that the two substance interact in a way to effectuate activity of one of the substances on, or to interact with, the other (e.g., an enzyme acting upon a sample). The term is not to be construed to require physical contact between the two substances, but further does not prohibit physical contact either. For example, proximity may be sufficient to affect the interaction and/or activity of the substances with one another. In some embodiments, contact is accomplished by introducing the substances into the same container (e.g., reaction vessel). In some embodiments contact is accomplished by introducing the substances into the same reaction vessel. In some embodiments, contact is accomplished by introducing substance A (e.g., reagent, dNTP, enzyme, etc.) into a reaction vessel, which either contains substance B (e.g., sample), to which substance B is simultaneously introduces, or to which substance B is later introduced. In some embodiments, contact is accomplished when substances physically touch one another (e.g., interact physically). In some embodiments, contact is accomplished when substances chemically interact with one another. In some embodiments, contact is accomplished when substances, enzymatically interact with one another. In some embodiments contact is accomplished when substances are proximal to one another.

In some embodiments, the polymerase is a DNA-dependent DNA polymerase. In some embodiments, wherein the polymerase has a strand-displacement activity. In some embodiments, the next-generation sequencing (NGS) is a short-read strategy. In some embodiments, the method comprises sequencing double-stranded DNA molecule by next-generation sequencing.

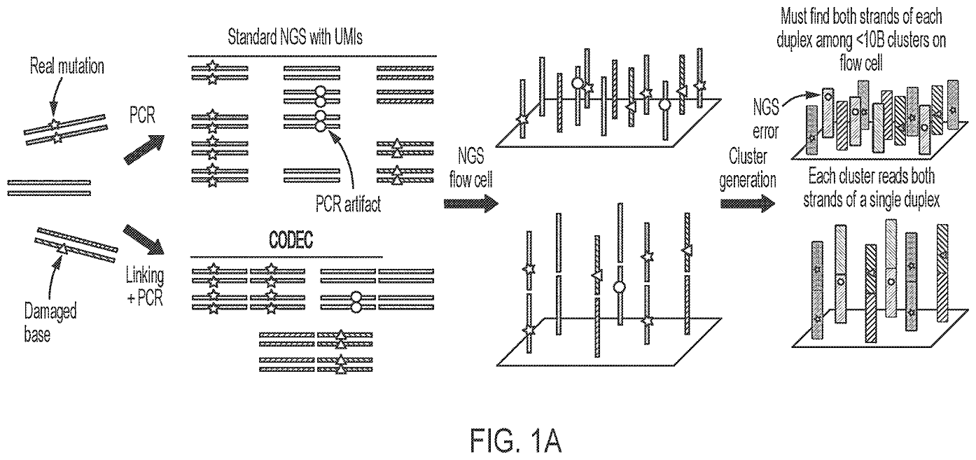

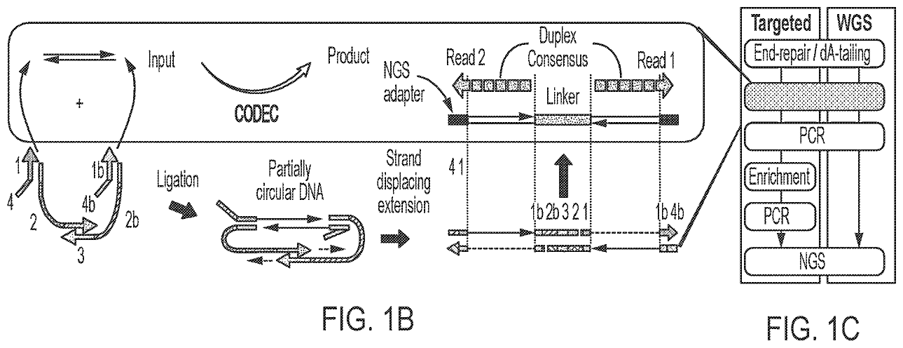

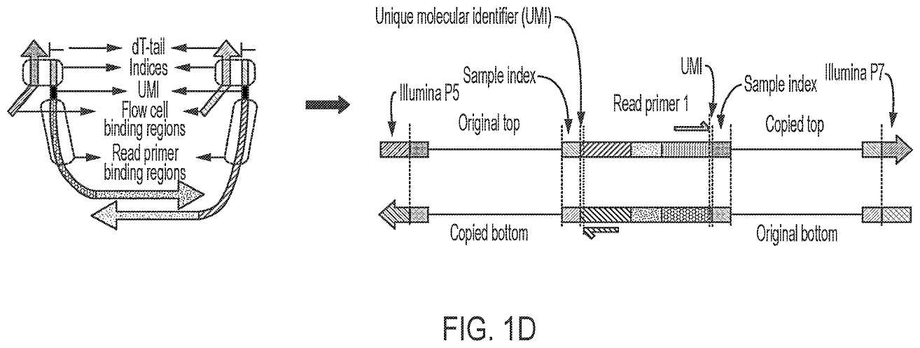

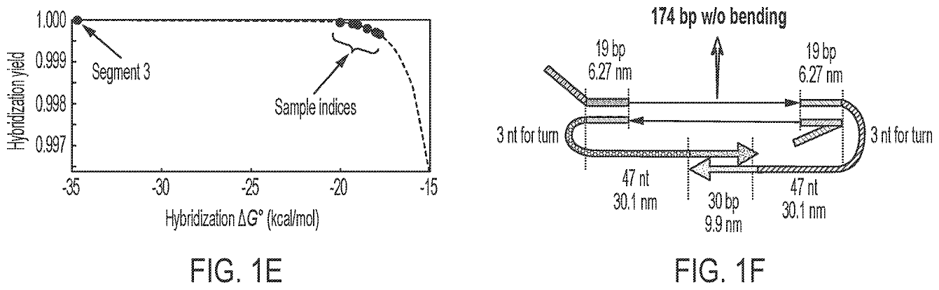

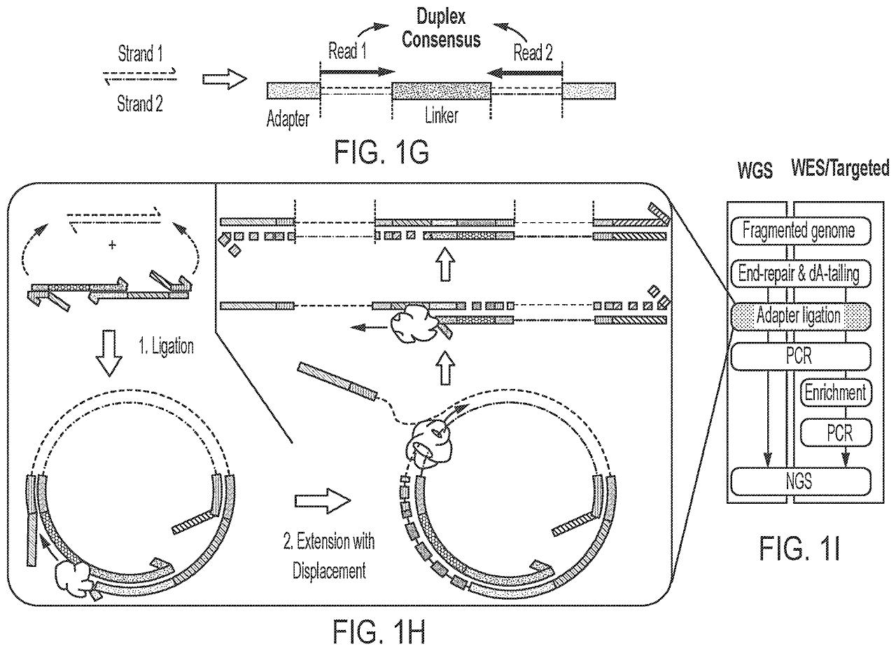

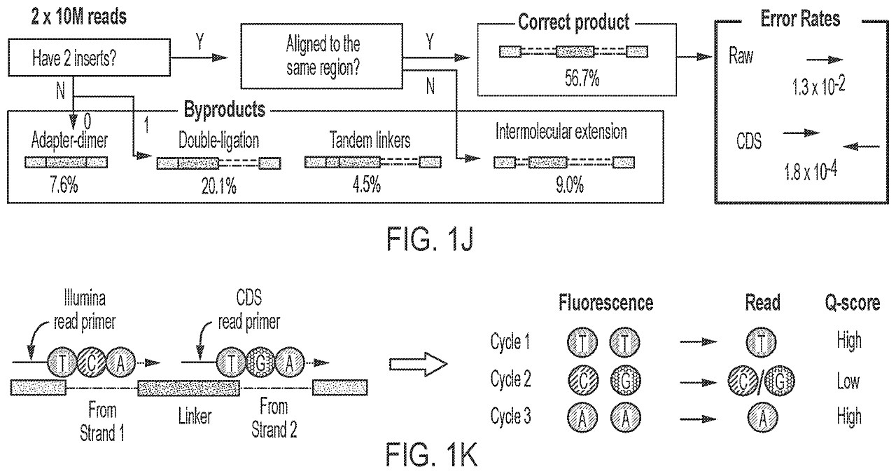

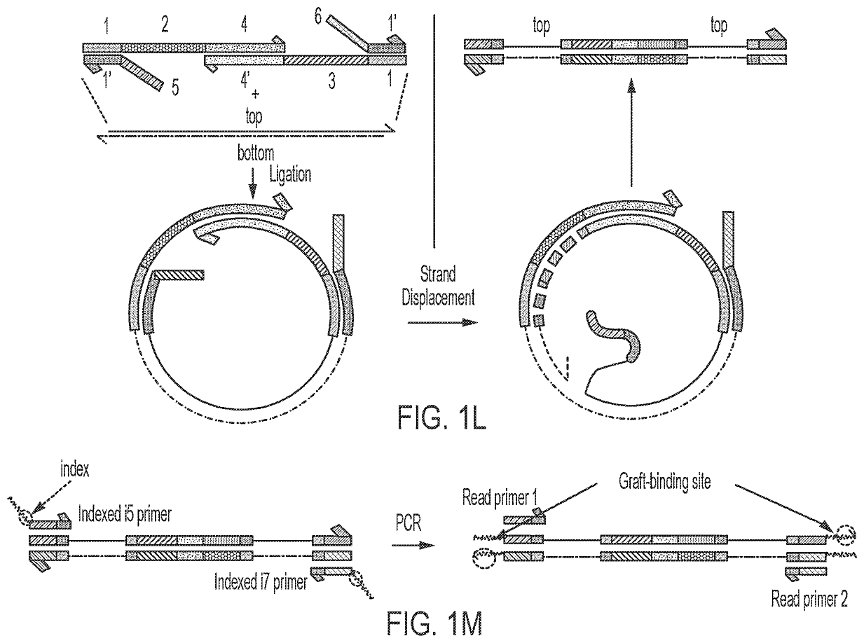

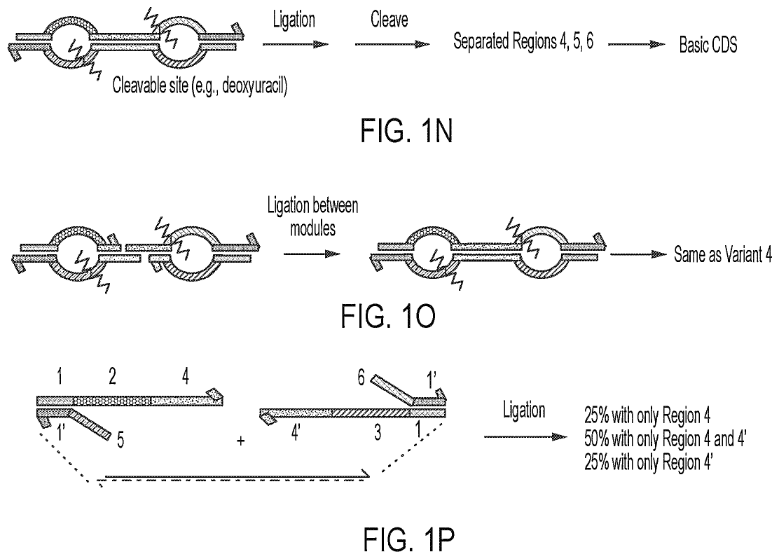

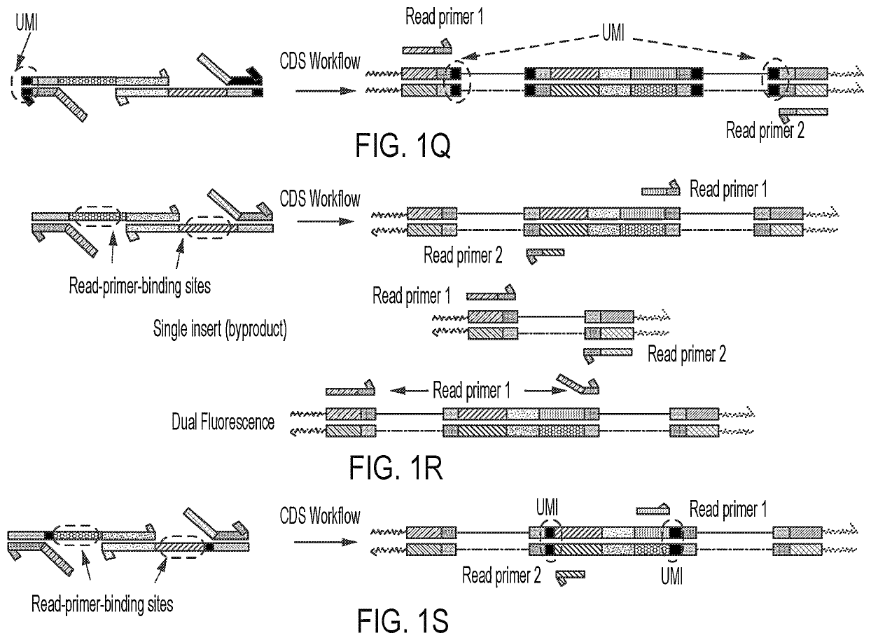

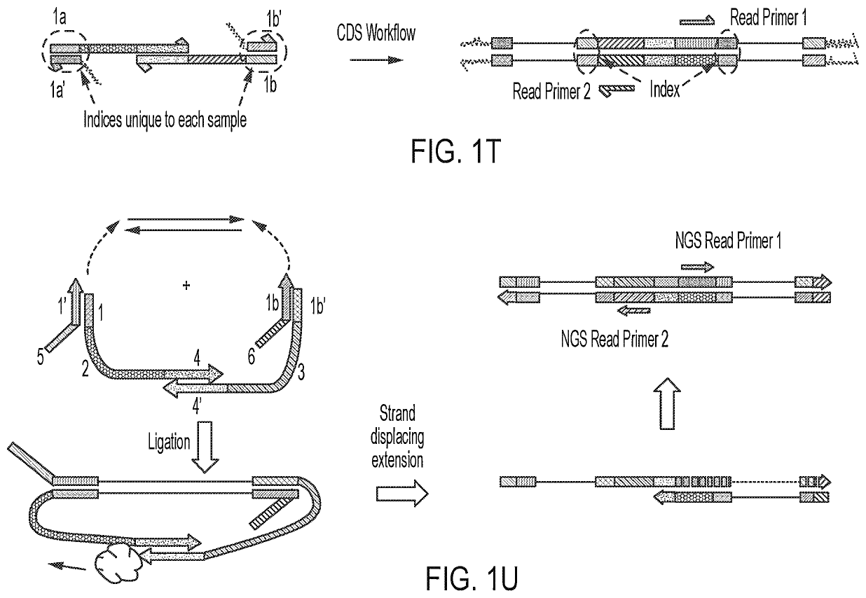

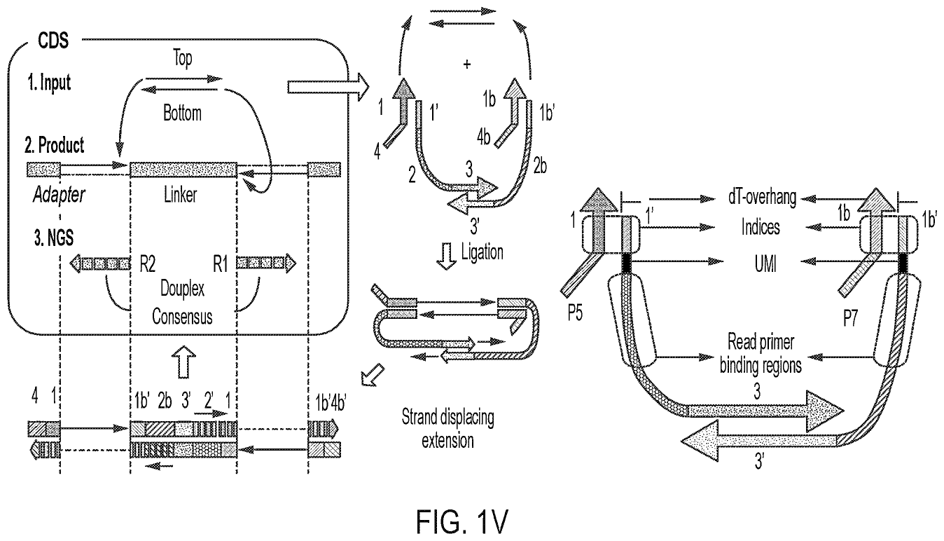

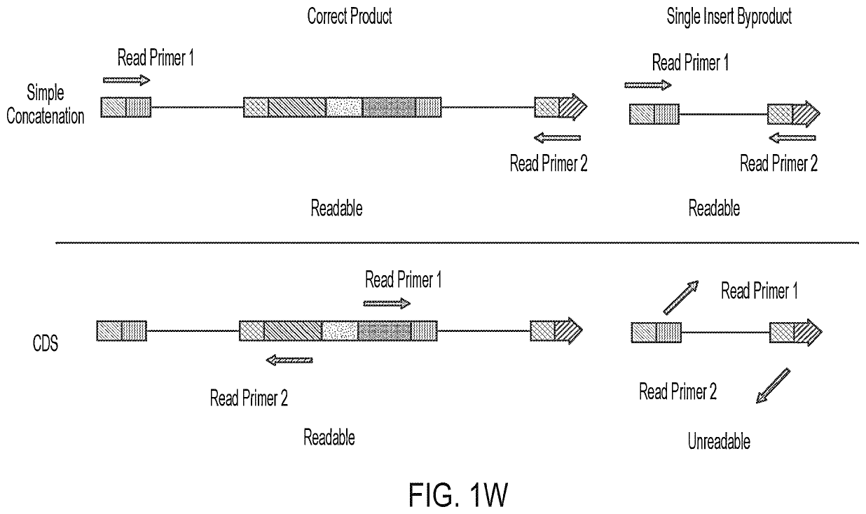

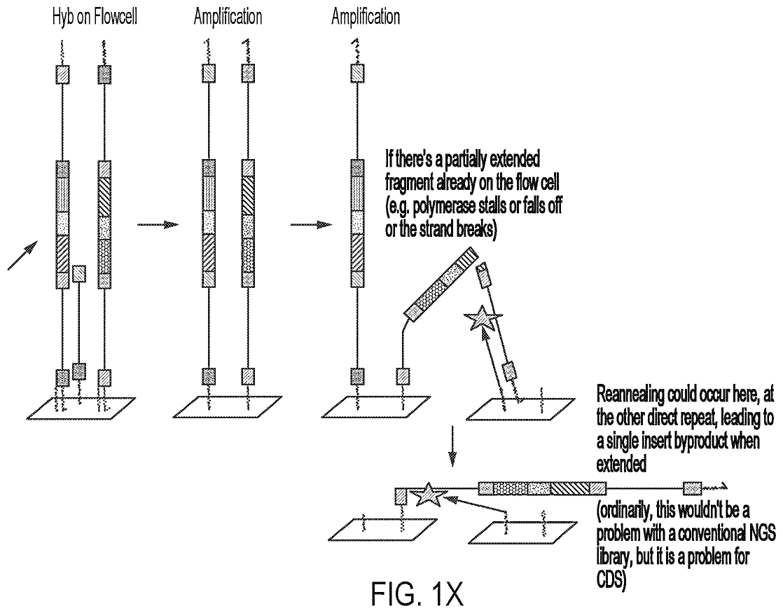

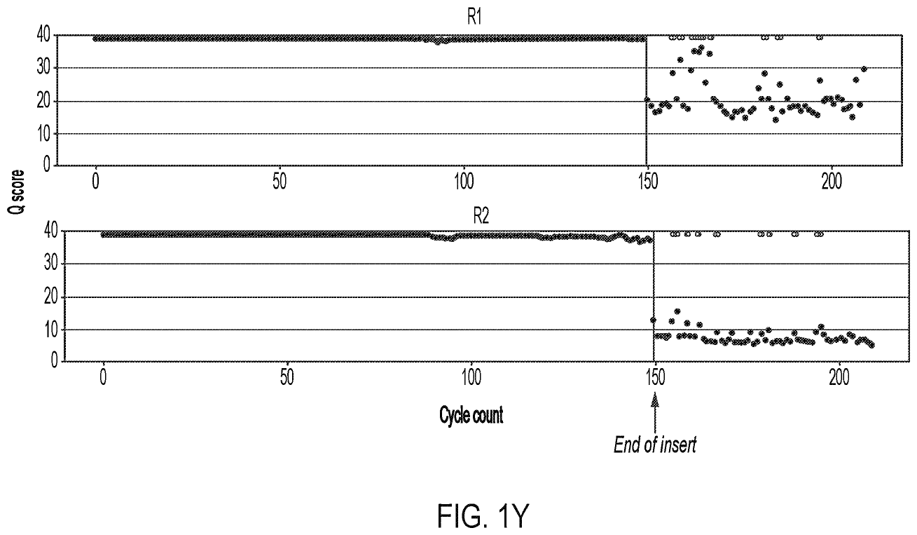

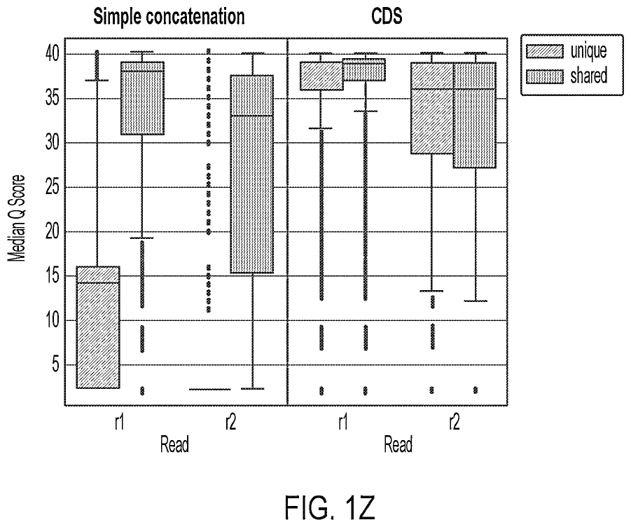

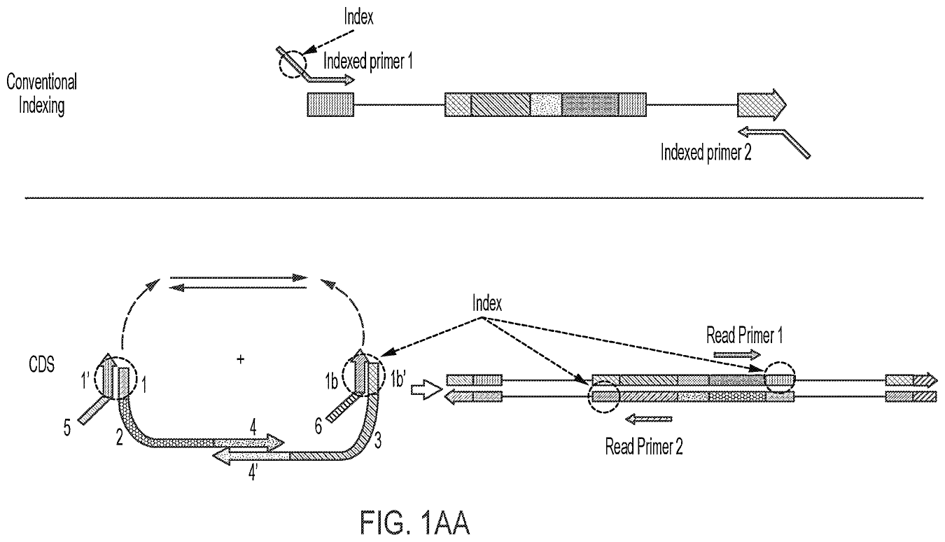

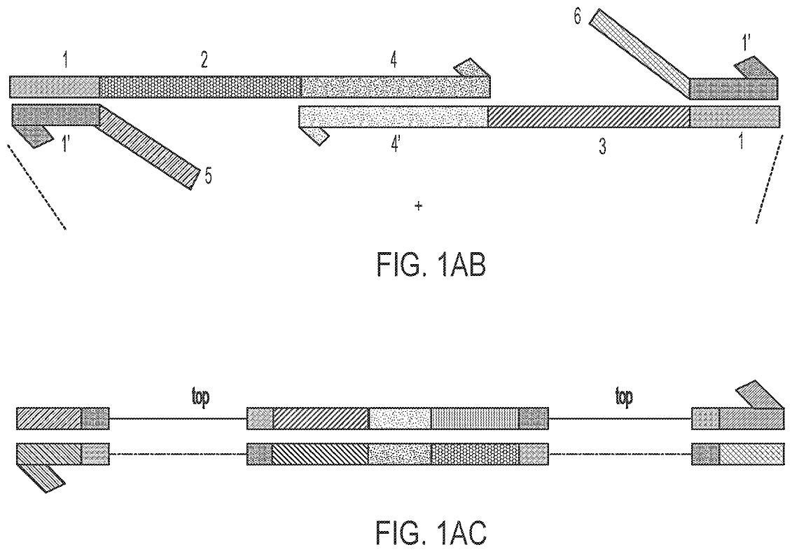

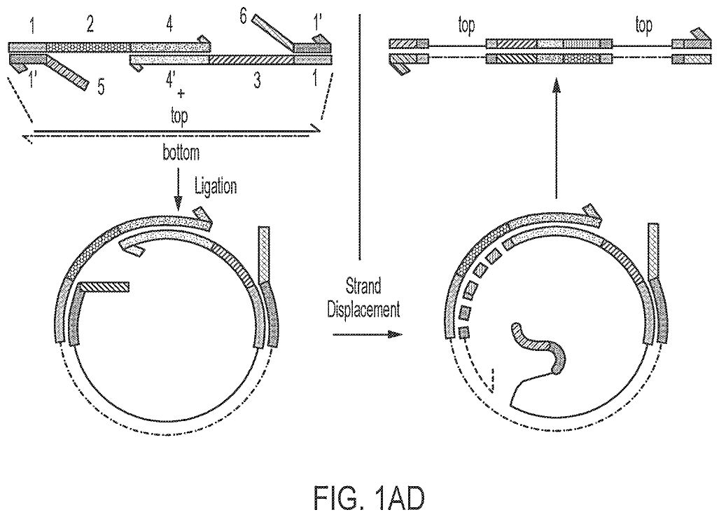

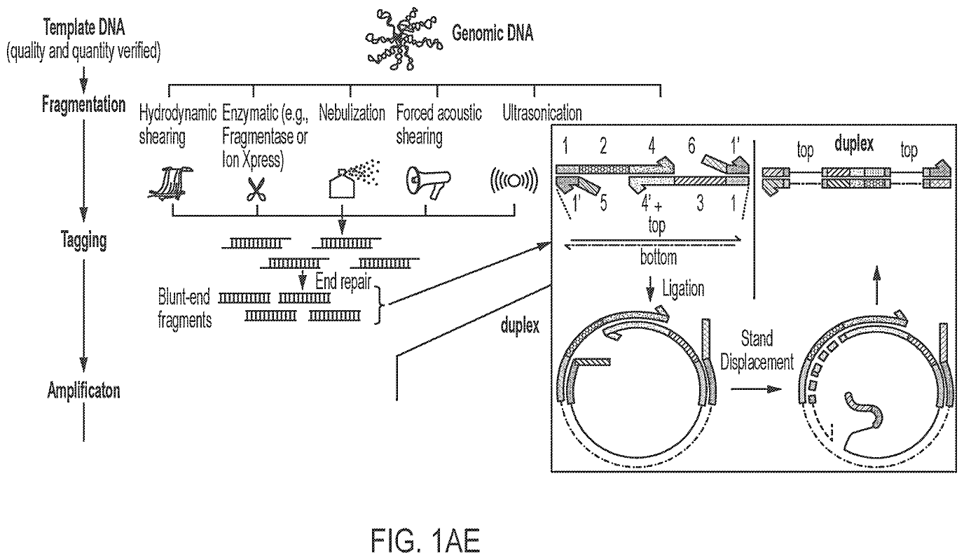

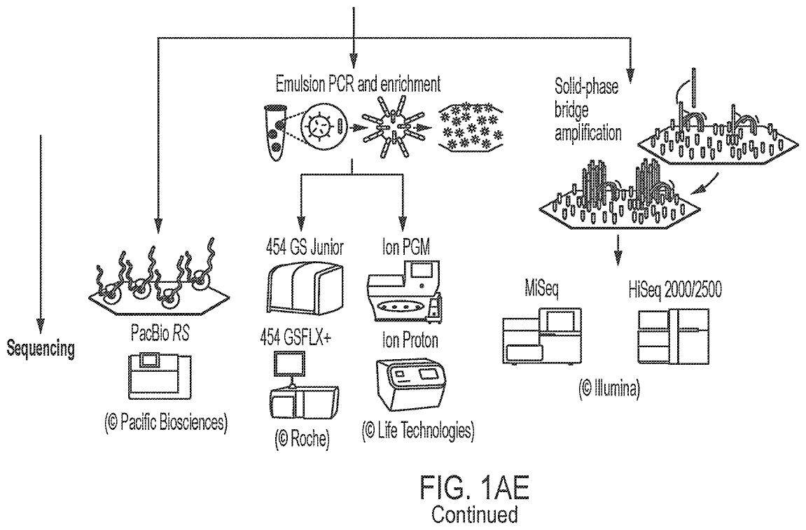

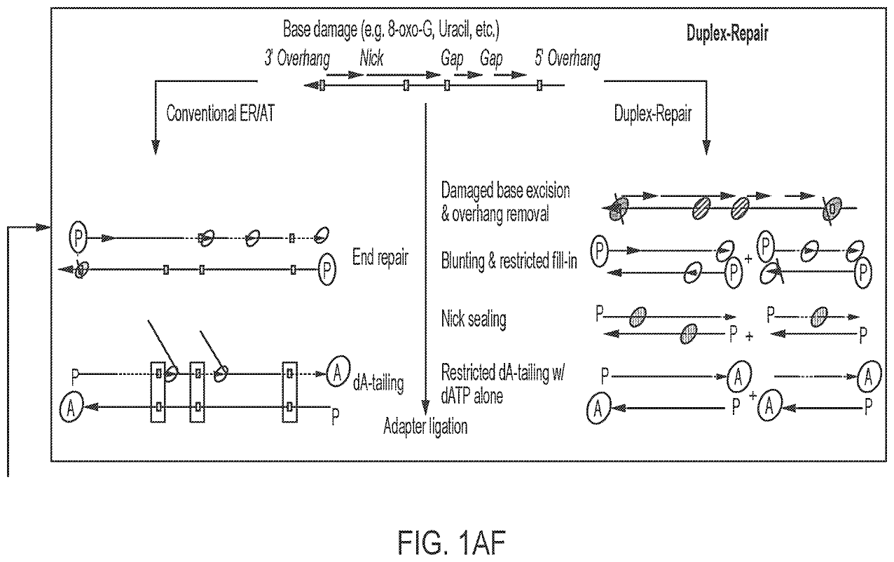

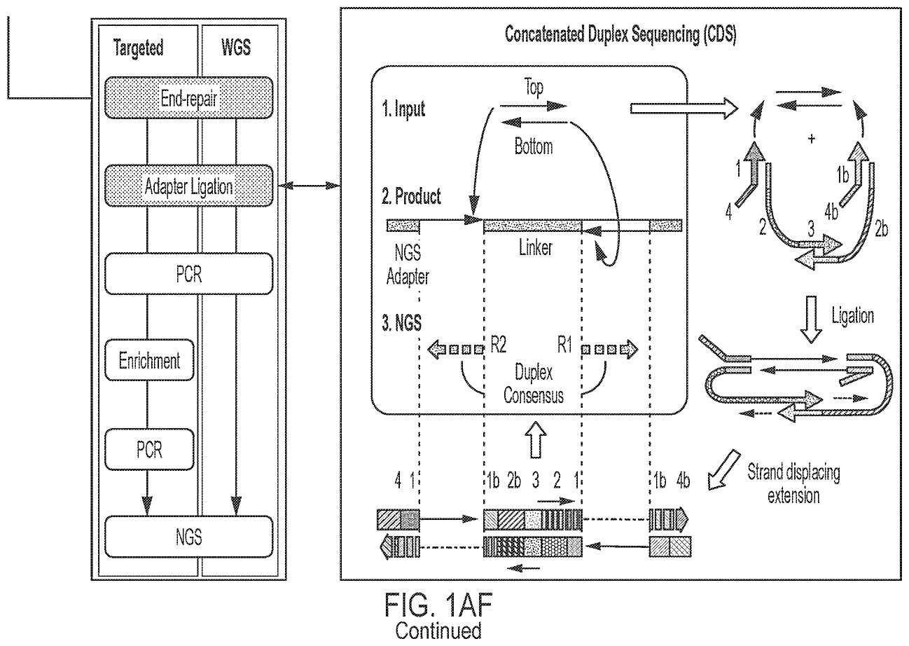

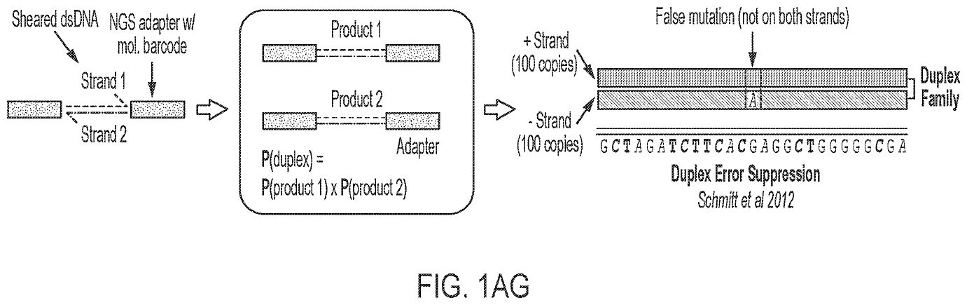

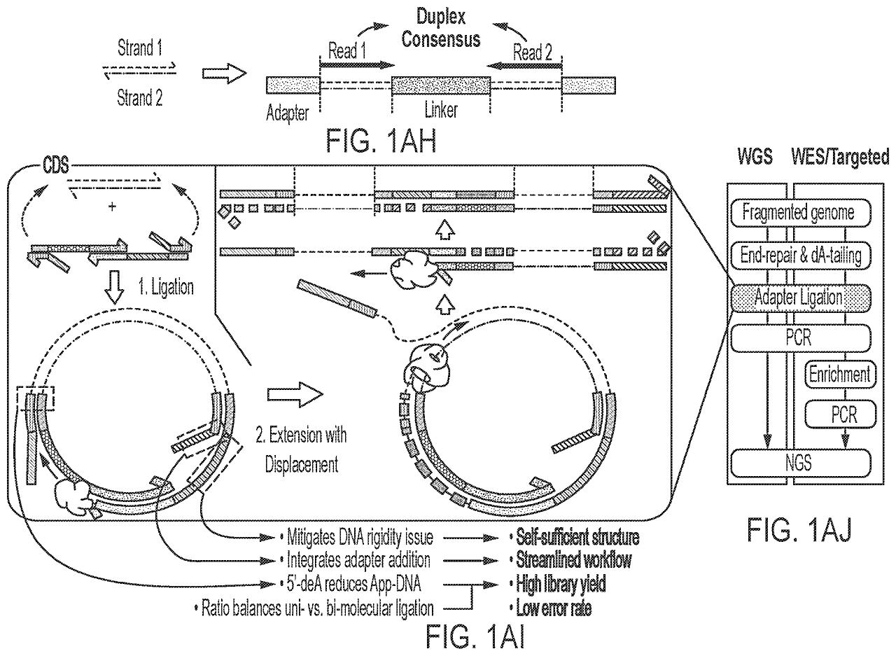

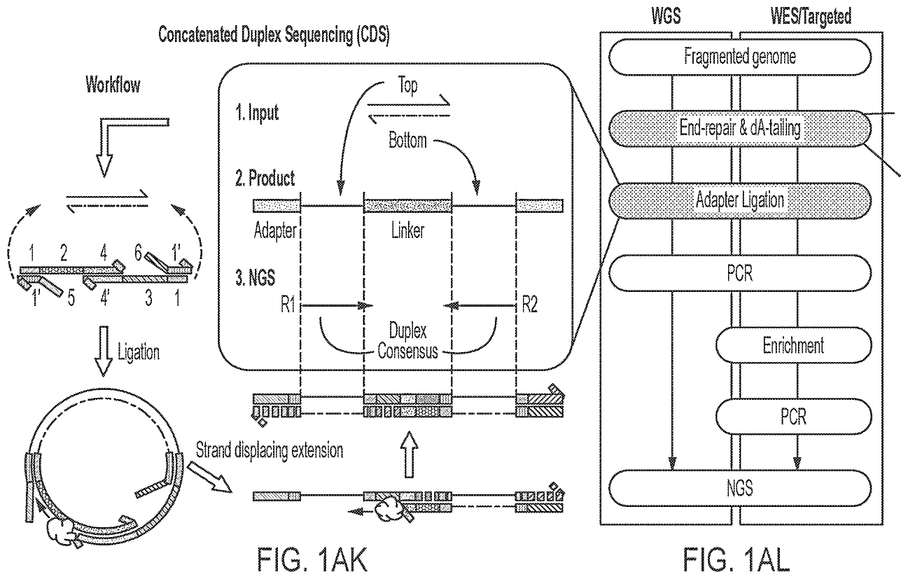

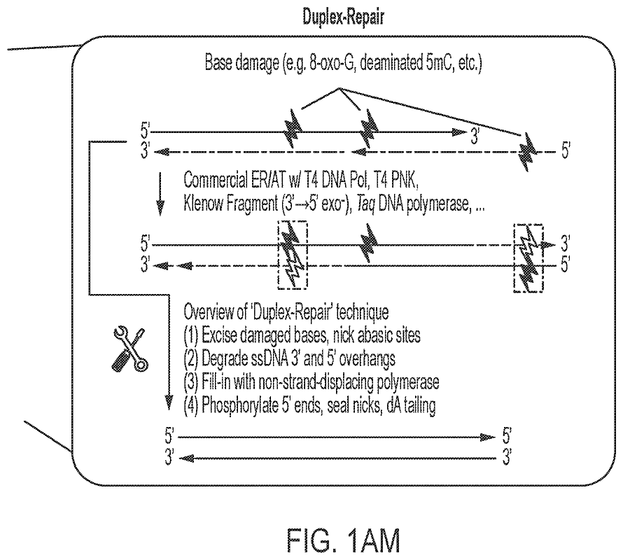

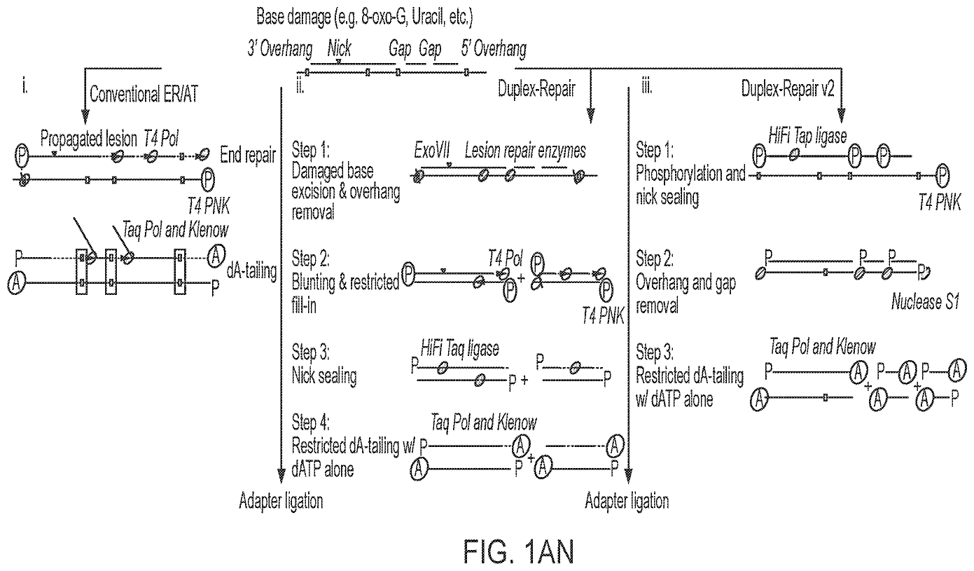

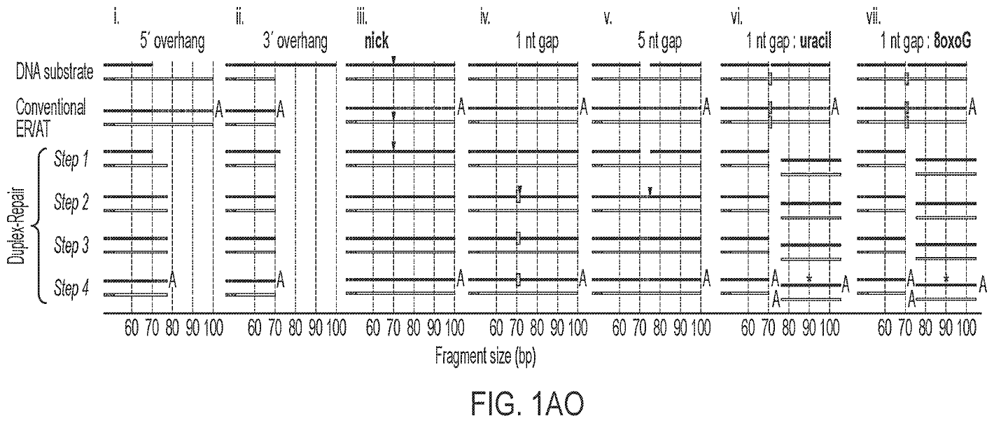

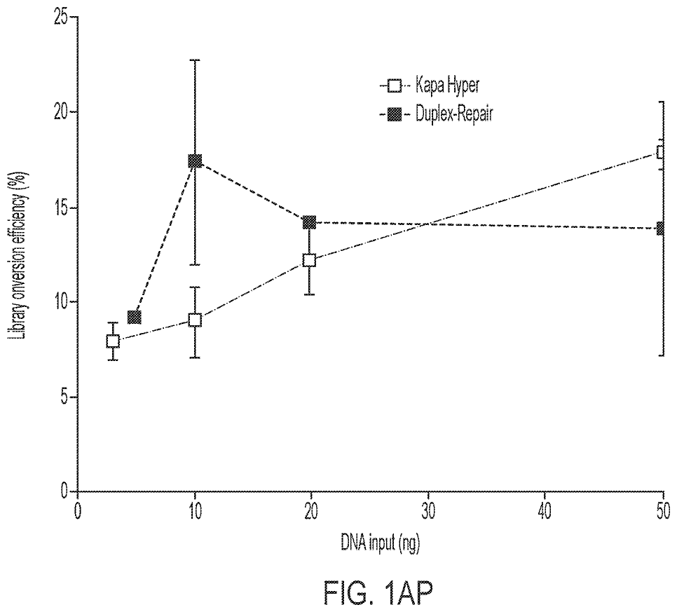

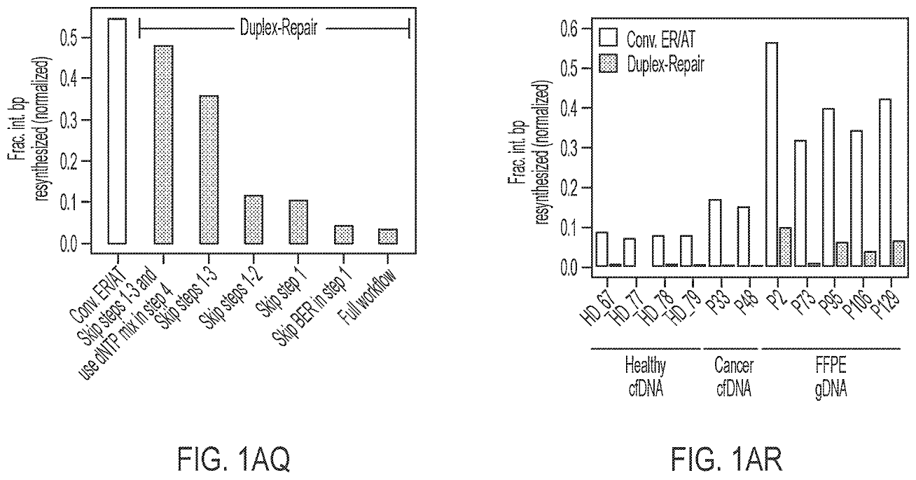

In some embodiments, the CODEC adapter sequence can be integrated to Illumina NGS library construction workflow by making R05 and R06 Illumina adapters (FIG. 1K). Indices are attached to demultiplex samples that have been pooled for NGS.

In other embodiments, the CODEC adapters described herein may include one or more modifications. Without limitation, the following represent modifications that may be used in connection with CODEC sequencing methods described herein:

1. Long duplex with mismatch bubbles

This variant, shown in FIG. 1L, works the same as the basic version except it needs to be cleaved to separate Regions 4, 5, and 6 after ligation. With only two oligos initially, it would be easier to hold all the components together.

2. Modular duplex with mismatch bubbles

This variant, shown in FIG. 1M, works the same as Variant 4 except it needs to be ligated first to assemble the intact adapters.

3. Half adapter complexes

Pre-annealing all four oligos isn't necessary for CDS. Annealing them into two half adapter complexes followed by ligation will theoretically result in 50% with both Region 4 and 4′. Once such structure is formed, Region 4 and 4′ will eventually hybridize with each other at some point during ligation or strand displacing extension (FIG. 1N).

4. UMI

Unique molecular identifiers (UMI) can be introduced at ligation sites as a part of Region 1 (FIG. 1O).

5A. Regions 2 and 3 as partial read primer binding sites

Although the main purpose of Regions 2 and 3 is adding flexibility for circularization, they can be repurposed to have other functions as well. FIG. 1P shows using them as partial read primer binding sites to read only correct products with Regions 2, 3, and 4.

This is because some byproducts have only a single insert just like conventional NGS samples, and utilizing Regions 2 and 3 prevents them from hybridizing with read primers. (FIG. 1P, “Single insert (byproduct)”).

However, both regular CDS adapter and this variant 5A may suffer from having two sites in a strand where the 3′-end of a read primer can hybridize (FIG. 1P, “Dual Fluorescence”). This can cause two different primers to generate dual fluorescence, which complicates data analysis. The variant shown in FIG. 1Q solves this issue.

5B. Regions 2 and 3 as complete read primer binding sites

This variant addresses the dual fluorescence issue by moving read primer binding sites completely into Regions 2 and 3 (FIG. 1Q). The read primers now don't hybridize with Region 1, so their 3′-end sequences are unique.

Another advantage of this version is the low cost of introducing UMI. Both regular NGS adapters and CDS adapters variant 1 need it at the end of double-stranded adapter regions before ligation with a target fragment. If UMI is 3 bp long, 43=64 pairs of adapter oligos have to be synthesized and annealed separately to avoid any UMI mismatch, which is expensive in terms of money and time. This variant can place UMI in single-stranded Regions 2 and 3 to avoid this requirement. With mixed bases at UMI positions, any length of UMI can be synthesized in a single batch.

Because the new read primer binding regions do not overlap with Region 1, base diversity at each sequencing cycle will be low if every read enters Region 1 at the same time. This can be solved by mixing four oligos with different lengths of UMI or using the next variant.

6. Region 1 as indices

An adapter complex doesn't necessarily have the same Region 1 on both sides; there can be independent Region 1a and 1b (FIG. 1R). Combined with the variant 5B, this variant can use Region 1a and 1b as sample indices, eliminating needs for indexed primers. This example directly attaches an index next to a target sequence to reduce cross-talk between samples known as index hopping.

Using Region 1 as indices can also address the base diversity issue mentioned earlier.

When multiple indices collectively have all four bases at every position, a pooled NGS library will get perfect base diversity throughout Region 1.

### (1) Overcoming “Mixed Clusters” to Achieve Highly-Accurate, Direct-Repeat Sequencing

Although successful concatenation of two strands may look sufficient for highly accurate and affordable NGS, byproducts comprising one strand (herein, referred as single-insert, SI) with the same adapter sequences on either end could form (FIG. 1U). The danger of this is two-fold: (1) if sequencing read primers are directed against the end adapter regions, forward and reverse reads from SI vs. CDS molecules would be difficult to discern, and (2) considering the high error rate from SI library molecules (0.1-1%), misclassification of even just a small fraction of SI reads as CDS reads could be detrimental.

It has been found here that SI byproducts can form by three major mechanisms: (A) Phi29 extension if adapter ligation is incomplete, i.e., if not all four phosphodiester bonds form (e.g., FIG. 1S), (B) PCR jumping in library amplification, considering the homology between the direct repeat sequences in the CDS product, and (C) PCR jumping in bridge amplification on the flow cell (FIG. 1V). (A) and (B) can be mitigated in part by size selection prior to sequencing and requiring ‘evidence’ of the linker sequence, e.g., using long enough reads to detect it after the insert. However, neither are sufficient to address (C). Indeed, it has been discovered here that in bridge amplification of CDS fragments, mixed clusters are formed comprising the original CDS library molecule which seeded the cluster and SI byproducts generated from one or both of the direct repeat sequences (FIG. 1V). Considering the log-linear nature of bridge amplification from a single “seeding” library molecule, the proportions of (i) CDS molecule, to (ii) SI byproduct of “top” strand, to (iii) SI byproduct of “bottom” strand, could be skewed over several orders of magnitude. When NGS read primers are directed against end adapter regions, mixed fluorescence occurs for (i)-(iii) but it becomes challenging to discern which bases were truly present in top versus bottom strand of the original DNA duplex from which the CDS library molecule was derived (FIG. 1W).

The solution here is to place read primer binding sites in the linker region such that only CDS fragments are sequenced. Yet, by nature of the linking process, segments 1/1′ and 1b/1b′ of the CDS adapter (FIG. 1S) will be present in both CDS and SI byproducts (see FIG. 1U). Thus, to further ensure that SI byproducts will not be read, the NGS read primer binding sites were placed in the positions indicated in FIG. 1U, which originate from segments 2 and 3 of the adapter. This also means that the early cycles of each sequencing read will start in the brown and light green segments; and to ensure that these cycles are not wasted, they are used to encode sample indices and unique molecular identifiers for each DNA fragment. This has other unique advantages such as to mitigate index hopping as described in the next section, and encoding base diversity to improve cluster recognition and chastity filtration on the sequencer. The single-stranded segments also increase the product yield by introducing flexibility to the circularization process, which is otherwise limited by rigidity of double-stranded DNA. Most importantly, this solves the “mixed cluster” problem by ensuring that only CDS molecules are sequenced in each forward and reverse read pair (FIG. 1X).

### (2) Preventing the Misassignment of CDS Reads to the Wrong Samples

Another important feature of CDS is index hopping suppression to prevent sample misassignment. This is particularly important when seeking to rely upon single CDS reads to achieve duplex sequencing accuracy, as even just a small fraction of reads which are improperly assigned to the wrong samples could introduce large numbers of errors. The limitations of conventional indexing are tagging indices away from inserts and not tagging until PCR, which is the final step of sample preparation. Because indices are commonly placed towards the 5′ end of primers which target homologous regions of adapters, residual primers could easily ‘swap’ onto new library molecules and change the samples to which they are assigned. (The same could happen with partly extended library molecules, by way of PCR jumping.) To address this, CDS indices were placed within the adapter complex itself, which enables attaching indices right next to inserts as soon as adapter ligation (FIG. 1Y). Because reading an index and an insert is now seamless with a single read primer, there's much less chance of cross-talk among molecules during sequencing. Also, because CDS requires insert 1 to match insert 2, any PCR jumping which occurs in the insert or linker regions would be evident as it would create intermolecular byproducts with different insert 1 and insert 2 sequences. Of note, sufficient diversity was incorporated among CDS indices so as to ensure proper cluster generation and chastity filtration, given that indices are read in the early cycles of sequencing, in this configuration. The index read cycles were also “repurposed” towards read 1 and read 2, so as not to “waste” cycles by reading indices at the start of each read. Reading indices inline also has the benefit of minimizing cluster cross-talk which has been shown to occur when index sequences are separately read apart from the inserts.

Unless otherwise defined herein, scientific and technical terms used in connection with the present disclosure shall have the meanings that are commonly understood by those of ordinary skill in the art (e.g., the skilled artisan). The meaning and scope of the terms are clear; however, in the event of any latent ambiguity, definitions provided herein take precedent over any dictionary or extrinsic definition. Further, unless otherwise required by context, singular terms shall include pluralities and plural terms shall include the singular. In this disclosure, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics, and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present disclosure unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of subjects.

The term “downstream,” as may be used herein, refers to the location of a nucleotide in relation to a landmark in a given sequence of multiple nucleotides (e.g., a nucleic acid), such that downstream shall mean “more 3′” (in the case of a nucleic acid) than the landmark. For example, a nucleotide is downstream from a landmark if it is closer to the 3′ end (and thus further from the 5′ end) of the nucleic acid than the landmark. Conversely, the term “upstream,” as may be used herein, refers to the location of a nucleotide in relation to a landmark of a given sequence of multiple nucleotides (e.g., a nucleic acid), such that upstream shall mean “more 5′” (in the case of a nucleic acid) than the landmark. For example, a nucleotide is upstream from a landmark if it is closer to the 5′ end (and thus further from the 3′ end) of the nucleic acid than the landmark.

The terms “approximately” or “about,” as may be used interchangeably herein, and as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction of (i.e., percentage greater than or percentage less than) the stated reference value unless otherwise stated or otherwise evident from the context (for example, when such number would exceed 100% of a possible value).Percent Identity

The terms “percent identity,” “sequence identity,” “% identity,” “% sequence identity,” and % identical,” as they may be interchangeably used herein, refer to a quantitative measurement of the similarity between two sequences (e.g., nucleic acid or amino acid). The percent identity of genomic DNA sequence, intron and exon sequence, and amino acid sequence between humans and other species varies by species type, with chimpanzee having the highest percent identity with humans of all species in each category.

Calculation of the percent identity of two nucleic acid sequences, for example, can be performed by aligning the two sequences for optimal comparison purposes (e.g., gaps can be introduced in one or both of a first and second nucleic acid sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Atschul, S. F. et al., J. Molec. Biol., 215, 403 (1990)).

When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%,at least 96%, at least 96.5%,at least 97%, at least 97.5%,at least 98%, at least 98.5%,at least 99%, at least 99.5%, at least 99.6%, at least 99.7%, at least 99.8%, at least 99.9% identity) and all increments thereof (e.g., tenths of a percent (e.g., 0.1%), hundredths of a percent (e.g., 0.01%), etc.).

The term “substantially,” as may be used herein, when used to describe the degree or abundance of an activity, generally refers to the value of the activity as being an amount which is achievable without undue effort. As can be appreciated, this amount may vary depending on the activity being performed, with simpler activities requiring a higher threshold and more complex activities requiring a lower threshold. For example, without limitation, when referring to substantially eliminating or removing reagents, dNTPs, or enzymes from a mixture, a substantial amount, may refer to 50% or more removal. In some embodiments, substantial refers to at least 50% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 99.95%, 99.99%, or more) and to all values of the variable that are within the experimental error (e.g., within the 95% confidence interval for the mean) or within +/−10% of the indicated value, whichever is greater. In some embodiments, substantially refers to at least 75% of the target being removed. In some embodiments, substantially refers to at least 80% of the target being removed. In some embodiments, substantially refers to at least 85% of the target being removed. In some embodiments, substantially refers to at least 90% of the target being removed. In some embodiments, substantially refers to at least 95% of the target being removed.

The terms “wild type” and “native,” as may be used interchangeably herein, are terms of art understood by skilled artisans and mean the typical form of an item, organism, strain, gene, or characteristic as it occurs in nature as distinguished from engineered, mutant, or variant forms.

### Combination of Duplex Repair and CODEC Sequencing

In certain embodiments, such as in the modified NGS workflow depicted in FIG. 1AF, the present disclosure provides sequencing methods that combine (a) duplex repair and (b) CODEC sequencing.

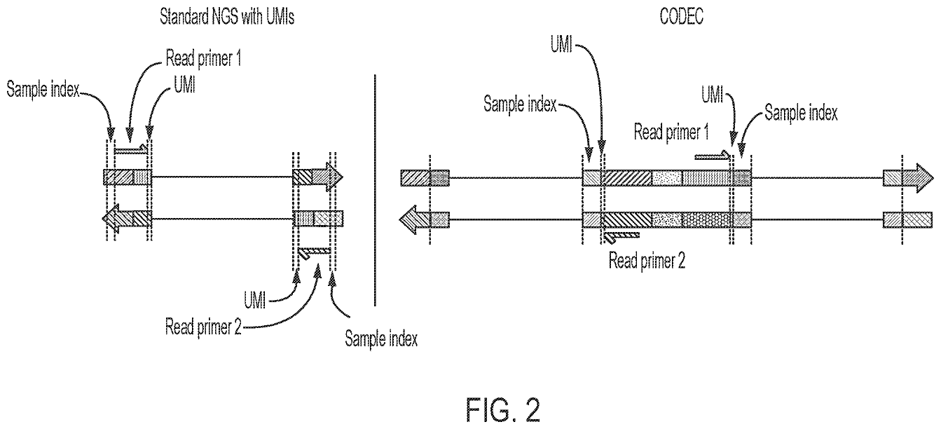

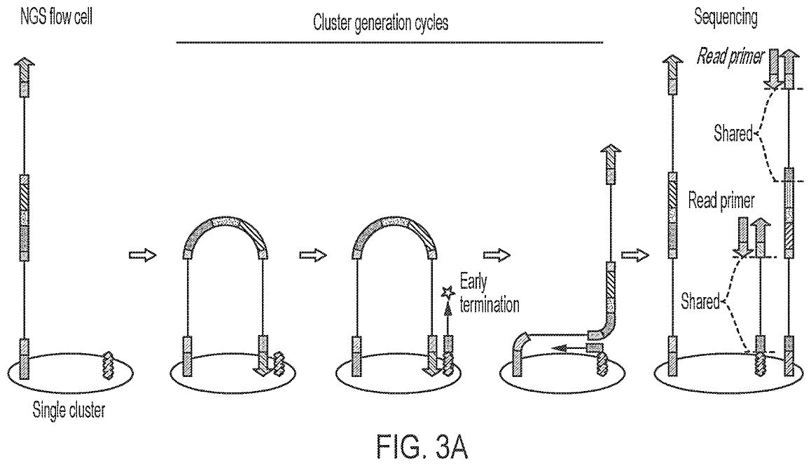

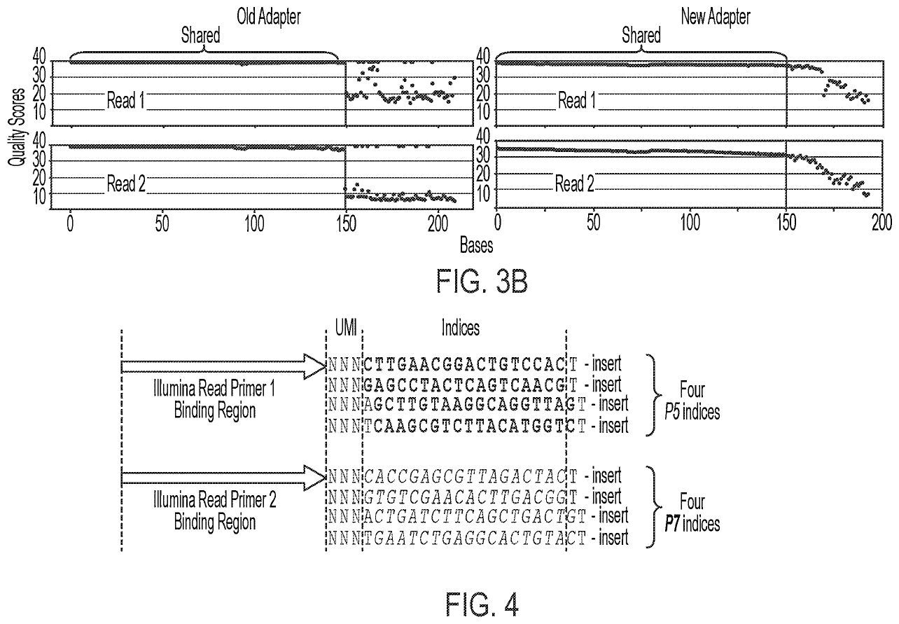

Existing methods used for nucleic acid preparation perform a number of activities and steps. The existing methods, known as “end repair” (ER) and “dA-tailing” (AT) (ER/AT), are used to blunt and phosphorylate DNA fragments, and perform non-templated addition of deoxyadenosine monophosphate (“dAMP”) to the 3′ ends, respectively, in preparation for ligation of dTMP-tailed sequencing adapters (FIG. 1). ER and AT are performed either sequentially or within a “one-pot” reaction (e.g., the entirety of the process and method occur concurrently within one reaction vessel without separation of steps), and employ DNA polymerase(s) which are intended to digest 3′ overhangs and fill-in 5′ overhangs, and to leave a single dAMP on each 3′ end of the strands of the duplex. Yet, ER/AT (either on its own, or in combination with pretreatments, such as NEB PreCR® or ExoVII—e.g., see FIG. 34 and FIGS. 35A-35C) traditionally involve the use of one or more DNA polymerase(s) which bear 5′ exonuclease and/or strand displacement activity. It was thus hypothesized that extensive strand resynthesis could occur from internal nicks and gaps within the duplex, and from long 5′ overhangs. If resynthesis occurs in the presence of an amplifiable lesion or alteration originally confined to one strand, it may, or is likely to, copy errors to both strands and render them indistinguishable from true mutations on both strands. While this source of false discovery in duplex sequencing is most clearly seen at fragment ends where short 5′ overhangs are often filled in (FIG. 2C), it is shown herein that such errors could also span much deeper into fragments given (i) the 5′ exonuclease and strand-displacement activities of polymerases such as Taq and Klenow which are commonly used in ER/AT and (ii) the varied extents of backbone damages, induced by multiple intrinsic or extrinsic factors, that serve as ‘priming sites’ for strand resynthesis (e.g., nicks, gaps). This could explain why a long tail of errors was observed that decreased with distance from 3′ fragment end in the heavily damaged FFPE tumor DNA samples, which had ˜100-fold higher error rates than 271 cell-free DNA (cfDNA) samples (FIG. 2C). This mechanism has also been confirmed through experiments involving treatment of synthetic oligonucleotides bearing nicks, gaps, and overhangs with traditional ER/AT kits (FIG. 2B and FIG. 3A). While errors at fragment ends can be mitigated through in silico trimming of fragment ends, errors which arise within the interior of each fragment (or, beyond a prespecified distance from fragment end, e.g., >12 bp) cannot be resolved in this manner without severely compromising the yield of DNA sequencing data. This means that while duplex sequencing can, in theory, discern base damage errors on one strand, its ability, in practice, depends on the quality of the starting material, which for a multitude of reasons, is deeply problematic. For example, prior to ER/AT, samples are fragmented to prepare a library. This fragmentation breaks apart a nucleic acid into small fragments. This can be accomplished, physically (e.g., by sonication or physical force), enzymatically, or chemically. However, all forms of fragmentation inherently damage the strands to break them and can induce off-target damage (e.g., overhangs, nicks, gaps, damaged bases).

Disclosed herein is a new ER/AT method called Duplex-Repair (DR), which minimizes and/or eliminates many of the problems inherent to existing methods. For example, without limitation, DR minimizes strand resynthesis prior to ligation of NGS adapters, which significantly limits false mutation discovery. As shown herein, by minimizing this resynthesis, DR addresses a major Achilles' heel of duplex sequencing, and other related methods, which rely upon a consensus of sequences from both strands of each duplex, to provide maximum accuracy and robustness.

In the embodiment shown in FIG. 1AF, a typical NGS workflow is shown in the lower left schematic and comprises (i) end-repair of a DNA sample to be sequenced, (ii) NGS adapter ligation, (iii) PCR amplification (e.g., flow cell cluster amplification), (iv) enrichment, (v) PCR, and (vi) sequencing by NGS. This figure is not intended to limit a NGS workflow to a specific workflow in the context of the present disclosure. Any NGS workflow (e.g., Ilumina NGS workflow) may be utilized. In the embodiment shown, the end-repair step is replaced by duplex repair and the adapter ligation step is replaced by CODEC disclosure provides a modified duplex repair may be used prior to conducting CODEC sequencing, a nucleic acid sample may be treated by the method of duplex repair (DR) in order to minimize propagation of false mutations, such as false mutations due to amplification of nucleotide damage or alterations originally natively located in only one strand.

Accordingly, in some aspects, the disclosure relates to a method of preparing a nucleic acid sample (sample; and as such term is further elaborated upon herein) for sequencing that minimizes propagation of false mutations due to amplification of nucleotide damage or alterations originally natively located in only one strand, wherein at least a portion of the sample is double-stranded, comprising adding a sample to a reaction vessel and: (a) contacting the sample to one or more enzymes capable of: (i) excising one or more damaged bases from the sample; (ii) cleaving one or more abasic sites, and processing the resulting ends to be compatible with extension by a DNA polymerase and ligation by a DNA ligase; (iii) and digesting 5′ overhangs; (b) contacting the sample with one or more of: (i) a DNA-dependent DNA polymerase lacking both strand displacement and 5′ exonuclease activity but capable of fill-in single-stranded segments of the sample and/or digesting 3′ overhangs of the sample; and (ii) an enzyme capable of phosphorylating the 5′ ends of the strands of the sample; and (c) contacting the sample with a DNA ligase capable of sealing nicks. In some embodiments, the methods of the present disclosure further comprise (d) preparing the sample for adapter ligation, wherein the preparing comprises: (i) adding deoxyadenosine monophosphate (dAMP) to the 3′ ends of the strands of the sample (dA-tailing); or (ii) optionally further blunting the ends of the sample.

In some aspects, a method comprises preparing a nucleic acid sample (sample) wherein at least a portion of the sample is double-stranded, comprising adding a sample to a reaction vessel and: (a) contacting the sample with one or more enzymes capable of: (i) phosphorylating the 5′ ends of the strands of the sample; adding a 3′ hydroxyl moiety to the 3′ ends of the strands of the sample; and (ii) sealing nicks; (b) contacting the sample with one or more of an enzyme capable of removing the 5′ and 3′ overhangs while also digesting gap regions to produce blunted duplexes; and (c) adding deoxyadenosine monophosphate (dAMP) to the 3′ ends of the strands of the sample (dA-tailing). In such a method, the need to excise damaged bases, to treat with ExoVII, or to fill gaps and short 5′ overhangs which were left after ExoVII treatment may be mitigated by the use of an enzyme (e.g., endonuclease (e.g., Nuclease SI)) to cleave single-stranded gap regions and cleave nucleotides present in overhang regions. In some embodiments, an enzyme used in step (a)(1) comprises: T4 polynucleotide kinase, HiFi Taq Ligase, or a combination thereof. In some embodiments, an enzyme used in step (b) is Nuclease SI.

The terms “endonuclease” and “nuclease,” as may be used herein, is a term of art known to the skilled artisan to refer generally to an enzyme that cleaves a phosphodiester bond or bonds within a polynucleotide chain (e.g., oligonucleotide, nucleic acid). Nucleases may be naturally occurring or genetically engineered. In some embodiments, an endonuclease is endonuclease IV (EndoIV). In some embodiments, an endonuclease is endonuclease VIII (EndoVIII). In some embodiments, a nuclease comprises Nuclease S1 (see for example, without limitation, thermofisher.com/order/catalog/product/EN0321#/EN0321; promega.com/products/cloning-and-dna-markers/molecular-biology-enzymes-and-reagents/s i-nuclease/?catNum=M5761; takarabio.com/products/cloning/modifying-enzymes/nucleases/sl-nuclease; and sigmaaldrich.com/US/en/product/SIGMA/N5661). Nuclease S1 degrades single-stranded nucleic acids, releasing 5′-phosphoryl mono- or oligonucleotides and may also cleave double-stranded DNA (dsDNA) at the single-stranded region caused by a nick, gap, mismatch, or loop.

By performing a method as described herein, the likelihood of the introduction of false mutations is substantially mitigated. For example, by using enzymes which first perform the excision of damaged bases and cleaving of abasic sites and processing of the resulting ends to be compatible with extension by a DNA polymerase and ligation by a DNA ligase from the sample, either the base will be excised in one strand and a gap will be created (where a complementary strand still exists at the excision point and forms a backbone for the duplex to remain intact), or a duplex/strand break will occur, thus creating two ‘daughter’ duplexes (where a complementary strand does not exist at the excision point and the duplex breaks apart into two smaller nucleic acids). A benefit, without limitation, of this step is to induce strand breaks in gap regions bearing damaged bases, as step (b) of the methods disclosed herein may involve using a DNA polymerase to fill-in gaps, whereas any damaged or mismatched bases on one strand of a fully duplexed region which is not resynthesized prior to adapter ligation could be resolved computationally with duplex sequencing if left uncorrected. Further, when these resultant duplexes (either intact or broken apart (e.g., where strand break occurs) are then exposed (e.g., contacted) to an enzyme capable of digesting 5′ overhangs, any 5′ overhangs would be substantially reduced in length, limiting their subsequent fill-in in step (b) to the very ends of the fragment. Then, when the resultant duplexes are exposed (e.g., contacted) to a DNA-dependent DNA polymerase lacking both strand displacement and 5′ exonuclease activity but capable of fill-in of single-stranded segments of the sample and digestion of 3′ overhangs, and a polynucleotide kinase, any short remaining 5′ overhangs which had not been fully digested in the prior step would be filled in to achieve a blunt end; any remaining 3′ overhangs would be digested to produce a blunt end; and any interior gaps (e.g., the small gaps produced by excision of damaged bases and cleaving of abasic sites, and longer gaps which may also exist in DNA fragments) would be filled up to the 5′ end of the downstream DNA segment. Next, when the resultant duplexes are exposed (e.g., contacted) to a DNA ligase capable of sealing nicks (preferably with minimal end-joining activity, so as to avoid chimera formation) any remaining nicks (e.g., those left after gap filling, among others inherently present in the sample) will be sealed, forming a continuous, blunted duplex. Then, when the resultant duplexes are exposed (e.g., contacted) to a DNA polymerase capable of performing non-templated extension (e.g., addition) of dAMP to the 3′ ends of the DNA duplex (e.g., dA-tailing), using DNA polymerases such as Taq or Klenow fragment which bear 5′ exonuclease and strand displacement activity, respectively, there will be substantially fewer ‘priming sites’ available for strand resynthesis. Further, if step (d) is performed under conditions which limit the addition of nucleotides other than dAMP (e.g., by substantially removing dNTPs prior to this step, or by providing dATP in extreme excess), the potential for strand resynthesis in this step can be substantially mitigated. This preserved information allows for greater accuracy and resolution of mutations.

The term “contacted,” as may be used herein, is used to describe the exposure of one substance (e.g., enzyme, reagent, dNTP) to another substance (e.g., sample, mixture), in an amount and with the intention that the two substance interact in a way to effectuate activity of one of the substances on, or to interact with, the other (e.g., an enzyme acting upon a sample). The term is not to be construed to require physical contact between the two substances, but further does not prohibit physical contact either. For example, proximity may be sufficient to affect the interaction and/or activity of the substances with one another. In some embodiments, contact is accomplished by introducing the substances into the same container (e.g., reaction vessel). In some embodiments contact is accomplished by introducing the substances into the same reaction vessel. In some embodiments, contact is accomplished by introducing substance A (e.g., reagent, dNTP, enzyme, etc.) into a reaction vessel, which either contains substance B (e.g., sample), to which substance B is simultaneously introduces, or to which substance B is later introduced. In some embodiments, contact is accomplished when substances physically touch one another (e.g., interact physically). In some embodiments, contact is accomplished when substances chemically interact with one another. In some embodiments, contact is accomplished when substances, enzymatically interact with one another. In some embodiments contact is accomplished when substances are proximal to one another.

In some embodiments, the methods of the disclosure further comprise: (d) preparing the sample for adapter ligation, wherein the preparing comprises: (i) adding deoxyadenosine monophosphate (dAMP) to the 3′ ends of the strands of the sample (dA-tailing); or (ii) blunting the ends of the sample. In some embodiments, dA-tailing comprises, contacting a sample with an enzyme capable of incorporating deoxyadenosine monophosphate (dAMP) to the 3′ end of a strand of the sample and contacting the sample with dNTPs. In some embodiments, enzymes and/or dNTPs used in steps (a)-(c) of the methods of the disclosure are substantially removed from the reaction vessel prior to dA-tailing. In some embodiments, dNTPs substantially comprise dATPs. In some embodiments, one or more (e.g., 1, 2, 3, 4, 5, or more, as representative of steps (a), (b), (c), (d), etc.) of the methods as disclosed herein are performed in a “one-pot” reaction wherein the steps are performed through sequential addition of enzymes and buffers to the same reaction vessel and adjusting reaction conditions (e.g., temperature). In some embodiments, steps are performed sequentially. In some embodiments, reagents and enzymes from the prior step are not removed from the mixture prior to proceeding with a subsequent step. In some embodiments, reagents and enzymes from the prior step are removed from the mixture prior to proceeding with a subsequent step. In some embodiments, one or more steps are performed in one reaction vessel. In some embodiments, one or more steps are performed in more than one reaction vessel (e.g., transferred at least at one time-point throughout a method).

### Combination of Duplex Pre-Amplification and CODEC

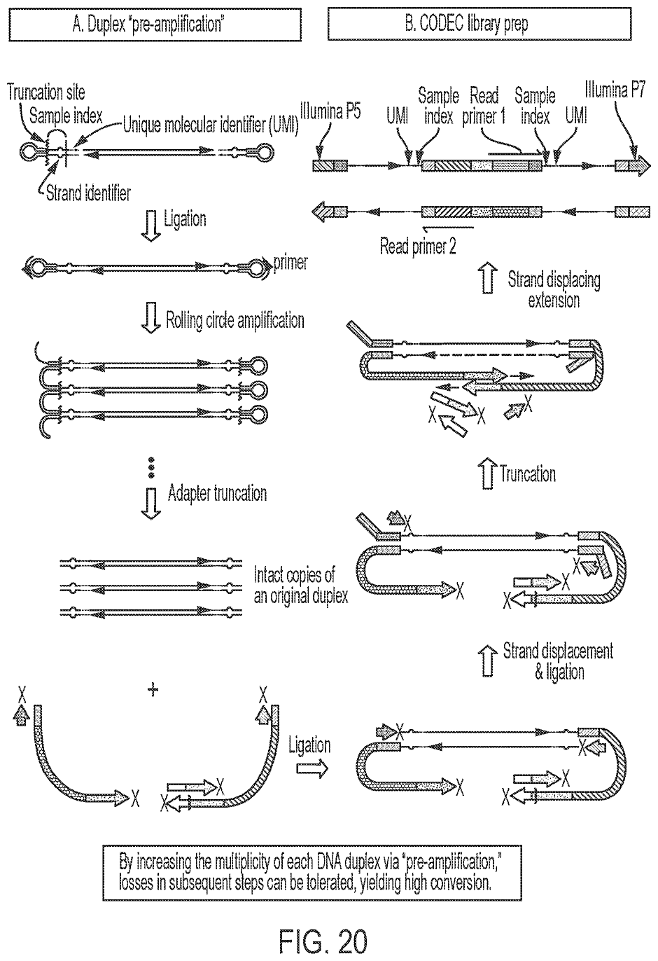

In various embodiments, duplex pre-amplification may be conducted on a nucleic acid sample (e.g., a DNA sample) prior to CODEC adapter ligation and CODEC sequencing. The nucleic acid samples described herein as input into CODEC sequencing may contain low-abundance nucleic acids. As such, the low-abundance nucleic acids may need to be amplified prior to CODEC adapter ligation and CODEC sequencing. Additionally, by amplifying nucleic acids prior to CODEC adapter ligation and CODEC sequencing, loss of nucleic acid material during CODEC adapter ligation and CODEC sequencing can be tolerated, thus yielding high conversion and high efficiency (FIG. 20).

In some embodiments, a nucleic acid within a nucleic acid sample is contacted with two pre-amplification molecules, each comprising a UMI, a sample index, a rolling circle amplification primer, and a truncation site. The term “rolling circle amplification,” as used herein, refers to a process of unidirectional nucleic acid replication that can rapidly synthesize multiple copies of a nucleic acid. The term “truncation site,” as used herein, refers to a nucleic acid site susceptible to cleavage. In some embodiments, a pre-amplification molecule is ligated to each end of a nucleic acid, allowing for rolling circle amplification of the nucleic acid, thus synthesizing multiple copies of the nucleic acid. In some embodiments, after rolling circle amplification and synthesis of multiple copies of the nucleic acid, the rolling circle amplification adapters comprising the rolling circle amplification primers are cleaved at the truncation sites, resulting in multiple copies of the same nucleic acid molecule. In some embodiments, after rolling circle amplification, the resulting plurality of nucleic acid molecules each comprise a sample index and a UMI. In some embodiments, the resulting plurality of nucleic acid molecules are ligated to a CODEC adapter and continue through the CODEC library preparation protocol and subsequent sequencing.

### Modified Method of CODEC Sequencing

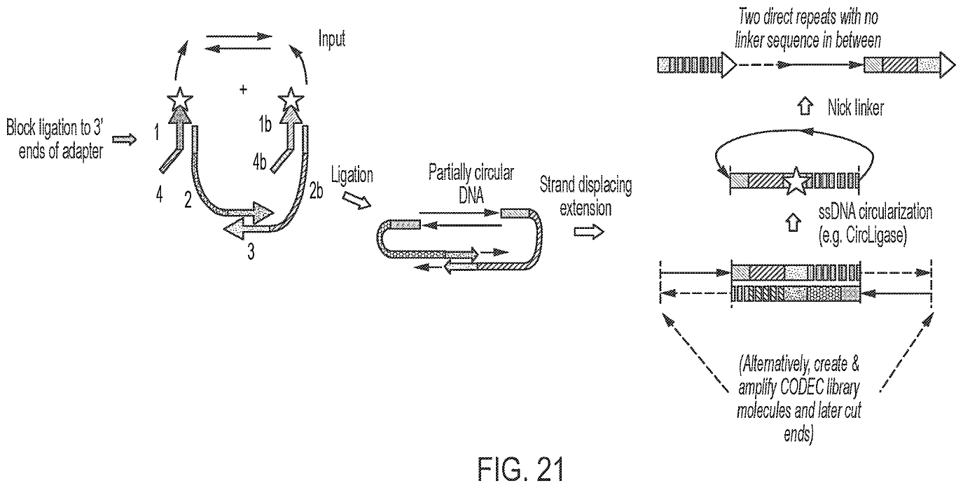

In some embodiments, CODEC sequencing may be conducted with a modified CODEC sequencing adapter (FIG. 21). In some embodiments, a standard CODEC sequencing adapter, as described herein, comprises read primers adjacent to a linker sequence at the middle of the CODEC sequencing adapter. In some embodiments, a modified CODEC sequencing adapter comprises read primers on the ends of the CODEC sequencing adapter and does not comprise a linker sequence at the middle of the modified CODEC sequencing adapter. In some embodiments, the modified CODEC sequencing adapter is produced following a method similar to the method used to produce the standard CODEC sequencing adapter and as described herein. In some embodiments, before the modified CODEC sequencing adapter is ligated to input dsDNA duplexes, the 3′ ends of the modified CODEC sequencing adapter are blocked from ligation. In some embodiments, after blocking of the 3′ ends of the modified CODEC sequencing adapter, the modified CODEC sequencing adapter is ligated to an input dsDNA duplex forming a partially circular DNA molecule. In some embodiments, the partially circular DNA molecule undergoes strand displacing extension, thus producing a linear modified CODEC sequencing molecule comprising the dsDNA duplex. In the alternative, the modified CODEC sequencing adapter is produced and ligated to an input dsDNA duplex, following the method used for producing the standard CODEC sequencing adapter and ligating the standard CODEC sequencing adapter to an input dsDNA duplex, but after strand displacing extension, the ends of the linear standard CODEC sequencing adapter are truncated, thus producing the modified CODEC sequencing molecule comprising the dsDNA duplex. In some embodiments, the modified CODEC sequencing molecule comprising the dsDNA duplex undergoes single-stranded DNA circularization. In some embodiments, the linker at the middle of the modified CODEC sequencing molecule is nicked, thus producing a linear CODEC sequencing molecule comprising both strands of the dsDNA duplex and read primers on both ends of the CODEC sequencing molecule. In some embodiments, the CODEC sequencing molecule comprises a linker at the middle of the CODEC sequencing molecule that is no more than one nucleotide in length. In some embodiments, the modified CODEC sequencing molecule can be sequenced following the same sequencing protocol as used for the standard CODEC sequencing molecule.

### Nucleic Acid Samples

In various aspects, the CODEC sequencing methods for sequencing DNA involve obtaining samples of nucleic acid molecules for sequence. Nucleic acid generally is acquired from a sample or a subject. Target molecules for labeling and/or detection according to the methods of the invention include, but are not limited to, genetic and proteomic material, such as DNA, genomic DNA, RNA, expressed RNA and/or chromosome(s). Methods of the invention are applicable to DNA from whole cells or to portions of genetic or proteomic material obtained from one or more cells. Methods of the invention allow for DNA or RNA to be obtained from non-cellular sources, such as viruses. For a subject, the sample may be obtained in any clinically acceptable manner, and the nucleic acid templates are extracted from the sample by methods known in the art. Generally, nucleic acid can be extracted from a biological sample by a variety of techniques such as those described by Maniatis, et al. (Molecular Cloning: A Laboratory Manual, Cold Spring Harbor, N.Y., pp. 280-281, 1982), the contents of which are incorporated by reference herein in their entirety.

Nucleic acid templates include deoxyribonucleic acid (DNA) and/or ribonucleic acid (RNA). Nucleic acid templates can be synthetic or derived from naturally occurring sources. Nucleic acids may be obtained from any source or sample, whether biological, environmental, physical, or synthetic. In one embodiment, nucleic acid templates are isolated from a sample containing a variety of other components, such as proteins, lipids and non-template nucleic acids. Nucleic acid templates can be obtained from any cellular material, obtained from an animal, plant, bacterium, fungus, or any other cellular organism. Samples for use in the present invention include viruses, viral particles or preparations. Nucleic acid may also be acquired from a microorganism, such as a bacteria or fungus, from a sample, such as an environmental sample.

In the present invention, the target material is any nucleic acid, including DNA, RNA, cDNA, PNA, LNA and others that are contained within a sample. Nucleic acid molecules include deoxyribonucleic acid (DNA) and/or ribonucleic acid (RNA). Nucleic acid molecules can be synthetic or derived from naturally occurring sources. In one embodiment, nucleic acid molecules are isolated from a biological sample containing a variety of other components, such as proteins, lipids and non-template nucleic acids. Nucleic acid template molecules can be obtained from any cellular material, obtained from an animal, plant, bacterium, fungus, or any other cellular organism. In certain embodiments, the nucleic acid molecules are obtained from a single cell. Biological samples for use in the present invention include viral particles or preparations. Nucleic acid molecules can be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid molecules can also be isolated from cultured cells, such as a primary cell culture or a cell line. The cells or tissues from which template nucleic acids are obtained can be infected with a virus or other intracellular pathogen. In addition, nucleic acids can be obtained from non-cellular or non-tissue samples, such as viral samples, or environmental samples.

A sample can also be total RNA extracted from a biological specimen, a cDNA library, viral, or genomic DNA. In certain embodiments, the nucleic acid molecules are bound as to other target molecules such as proteins, enzymes, substrates, antibodies, binding agents, beads, small molecules, peptides, or any other molecule and serve as a surrogate for quantifying and/or detecting the target molecule. Generally, nucleic acid can be extracted from a biological sample by a variety of techniques such as those described by Sambrook and Russell, Molecular Cloning: A Laboratory Manual, Third Edition, Cold Spring Harbor, N.Y. (2001). Nucleic acid molecules may be single-stranded, double-stranded, or double-stranded with single-stranded regions (for example, stem- and loop-structures). Proteins or portions of proteins (amino acid polymers) that can bind to high affinity binding moieties, such as antibodies or aptamers, are target molecules for oligonucleotide labeling, for example, in droplets.

Nucleic acid templates can be obtained directly from an organism or from a biological sample obtained from an organism, e.g., from blood, urine, cerebrospinal fluid, seminal fluid, saliva, sputum, stool and tissue. In a particular embodiment, nucleic acid is obtained from fresh frozen plasma (FFP). In a particular embodiment, nucleic acid is obtained from formalin-fixed, paraffin-embedded (FFPE) tissues. Any tissue or body fluid specimen may be used as a source for nucleic acid for use in the invention. Nucleic acid templates can also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, viral, or genomic DNA.

A biological sample may be homogenized or fractionated in the presence of a detergent or surfactant. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is non-denaturing, can act to solubilize the sample. Detergents may be ionic or nonionic. Examples of nonionic detergents include triton, such as the Triton X series (Triton X-100 t-Oct-C6H4—(OCH2—CH2)xOH, x=9-10, Triton X-100R, Triton X-114 x=7-8), octyl glucoside, polyoxyethylene(9)dodecyl ether, digitonin, IGEPAL CA630 octylphenyl polyethylene glycol, n-octyl-beta-D-glucopyranoside (betaOG), n-dodecyl-beta, Tween 20 polyethylene glycol sorbitan monolaurate, Tween 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14E06), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammoniol-1-propanesulf-onate.

Lysis or homogenization solutions may further contain other agents, such as reducing agents. Examples of such reducing agents include dithiothreitol (DTT), beta.-mercaptoethanol, DTE, GSH, cysteine, cysteamine, tricarboxyethyl phosphine (TCEP), or salts of sulfurous acid. Once obtained, the nucleic acid is denatured by any method known in the art to produce single stranded nucleic acid templates and a pair of first and second oligonucleotides is hybridized to the single stranded nucleic acid template such that the first and second oligonucleotides flank a target region on the template.

In some embodiments, nucleic acids may be fragmented or broken into smaller nucleic acid fragments. Nucleic acids, including genomic nucleic acids, can be fragmented using any of a variety of methods, such as mechanical fragmenting, chemical fragmenting, and enzymatic fragmenting. Methods of nucleic acid fragmentation are known in the art and include, but are not limited to, DNase digestion, sonication, mechanical shearing, and the like (J. Sambrook et al., “Molecular Cloning: A Laboratory Manual”, 1989, 2.sup.nd Ed., Cold Spring Harbour Laboratory Press: New York, N.Y.; P. Tijssen, “Hybridization with Nucleic Acid Probes-Laboratory Techniques in Biochemistry and Molecular Biology (Parts I and II)”, 1993, Elsevier; C. P. Ordahl et al., Nucleic Acids Res., 1976, 3: 2985-2999; P. J. Oefner et al., Nucleic Acids Res., 1996, 24: 3879-3889; Y. R. Thorstenson et al., Genome Res., 1998, 8: 848-855). U.S. Patent Publication 2005/0112590 provides a general overview of various methods of fragmenting known in the art.

Genomic nucleic acids can be fragmented into uniform fragments or randomly fragmented. In certain aspects, nucleic acids are fragmented to form fragments having a fragment length of about 5 kilobases or 100 kilobases. In a preferred embodiment, the genomic nucleic acid fragments can range from 1 kilobases to 20 kilobases. Preferred fragments can vary in size and have an average fragment length of about 10 kilobases. However, desired fragment length and ranges of fragment lengths can be adjusted depending on the type of nucleic acid targets one seeks to capture. The particular method of fragmenting is selected to achieve the desired fragment length. A few non-limiting examples are provided below.

Chemical fragmentation of genomic nucleic acids can be achieved using a number of different methods. For example, hydrolysis reactions including base and acid hydrolysis are common techniques used to fragment nucleic acid. Hydrolysis is facilitated by temperature increases, depending upon the desired extent of hydrolysis. Fragmentation can be accomplished by altering temperature and pH as described below. The benefit of pH-based hydrolysis for shearing is that it can result in single-stranded products. Additionally, temperature can be used with certain buffer systems (e.g. Tris) to temporarily shift the pH up or down from neutral to accomplish the hydrolysis, then back to neutral for long-term storage etc. Both pH and temperature can be modulated to affect differing amounts of shearing (and therefore varying length distributions).

Chemical cleavage can also be specific. For example, selected nucleic acid molecules can be cleaved via alkylation, particularly phosphorothioate-modified nucleic acid molecules (see, e.g., K. A. Browne, “Metal ion-catalyzed nucleic Acid alkylation and fragmentation,” J. Am. Chem. Soc. 124(27): 7950-7962 (2002)). Alkylation at the phosphorothioate modification renders the nucleic acid molecule susceptible to cleavage at the modification site. See I. G. Gut and S. Beck, “A procedure for selective DNA alkylation and detection by mass spectrometry,” Nucl. Acids Res. 23(8): 1367-1373 (1995).

Methods of the invention also contemplate chemically shearing nucleic acids using the technique disclosed in Maxam-Gilbert Sequencing Method (Chemical or Cleavage Method), Proc. Natl. Acad. Sci. USA. 74:560-564. In that protocol, the genomic nucleic acid can be chemically cleaved by exposure to chemicals designed to fragment the nucleic acid at specific bases, such as preferential cleaving at guanine, at adenine, at cytosine and thymine, and at cytosine alone.

Mechanical shearing of nucleic acids into fragments can occur using any method known in the art. For example, fragmenting nucleic acids can be accomplished by hydroshearing, trituration through a needle, and sonication. See, for example, Quail, et al. (Nov 2010) DNA: Mechanical Breakage. In: eLS. John Wiley & Sons, Chichester.

The nucleic acid can also be sheared via nebulization, see (Roe, BA, Crabtree. JS and Khan, A S 1996); Sambrook & Russell, Cold Spring Harb Protoc 2006. Nebulizing involves collecting fragmented DNA from a mist created by forcing a nucleic acid solution through a small hole in a nebulizer. The size of the fragments obtained by nebulization is determined chiefly by the speed at which the DNA solution passes through the hole, altering the pressure of the gas blowing through the nebulizer, the viscosity of the solution, and the temperature. The resulting DNA fragments are distributed over a narrow range of sizes (700-1330 bp). Shearing of nucleic acids can be accomplished by passing obtained nucleic acids through the narrow capillary or orifice (Oefner et al., Nucleic Acids Res. 1996; Thorstenson et al., Genome Res. 1995). This technique is based on point-sink hydrodynamics that result when a nucleic acid sample is forced through a small hole by a syringe pump.

In HydroShearing (Genomic Solutions, Ann Arbor, Mich., USA), DNA in solution is passed through a tube with an abrupt contraction. As it approaches the contraction, the fluid accelerates to maintain the volumetric flow rate through the smaller area of the contraction. During this acceleration, drag forces stretch the DNA until it snaps. The DNA fragments until the pieces are too short for the shearing forces to break the chemical bonds. The flow rate of the fluid and the size of the contraction determine the final DNA fragment sizes.

Sonication is also used to fragment nucleic acids by subjecting the nucleic acid to brief periods of sonication, i.e. ultrasound energy. A method of shearing nucleic acids into fragments by sonification is described in U.S. Patent Publication 2009/0233814. In the method, a purified nucleic acid is obtained placed in a suspension having particles disposed within. The suspension of the sample and the particles are then sonicated into nucleic acid fragments.

Enzymatic fragmenting, also known as enzymatic cleavage, cuts nucleic acids into fragments using enzymes, such as endonucleases, exonucleases, ribozymes, and DNAzymes. Such enzymes are widely known and are available commercially, see Sambrook, J. Molecular Cloning: A Laboratory Manual, 3rd (2001) and Roberts RJ (January 1980). “Restriction and modification enzymes and their recognition sequences,” Nucleic Acids Res. 8 (1): r63-r80. Varying enzymatic fragmenting techniques are well-known in the art, and such techniques are frequently used to fragment a nucleic acid for sequencing, for example, Alazard et al, 2002; Bentzley et al, 1998; Bentzley et al, 1996; Faulstich et al, 1997; Glover et al, 1995; Kirpekar et al, 1994; Owens et al, 1998; Pieles et al, 1993; Schuette et al, 1995; Smirnov et al, 1996; Wu & Aboleneen, 2001; Wu et al, 1998a.

The most common enzymes used to fragment nucleic acids are endonucleases. The endonucleases can be specific for either a double-stranded or a single stranded nucleic acid molecule. The cleavage of the nucleic acid molecule can occur randomly within the nucleic acid molecule or can cleave at specific sequences of the nucleic acid molecule. Specific fragmentation of the nucleic acid molecule can be accomplished using one or more enzymes in sequential reactions or contemporaneously.

Any of the above aspects and embodiments can be combined with any other aspect or embodiment as disclosed in the Summary, Drawings, and/or in the Detailed Description sections, including the below examples/embodiments.

### EXAMPLES

### Example 1—CODEC Sequencing!

Discovering extremely low-level mutations within a single double-stranded DNA molecule (a ‘single duplex’) is crucial to finding diagnostic [1], predictive [2], and prognostic [3] biomarkers, understanding cancer evolution [4] and somatic mosaicism [5], and studying infectious diseases [6] and aging [7]. Third generation sequencing technologies (e.g., PacBio, Oxford Nanopore Technologies) in principle make it possible to sequence each single DNA duplex in whole to resolve true mutations on both strands apart from false mutations on either strand, but, in practice, lack the required accuracy and throughput [8,9]. Next generation sequencing (NGS), on the other hand, continues to offer superior read accuracy and throughput [10], but is not configured to sequence single duplexes—at least not without severely compromising its throughput or utility.

NGS affords high throughput by reading short, clonally amplified DNA fragments in massively parallel fluorescence analysis. Yet, its accuracy is limited by the need to dissociate Watson and Crick strands of each DNA duplex. Without a complementary strand for comparison, errors introduced on either strand due to base damage, PCR, and sequencing [11] can be disguised as real mutations (FIG. 1A). While it is possible to use unique molecular identifiers (UMIs) to separately track both strands of each DNA molecule and compare their sequences to detect true mutations on both strands of each duplex [12], it does not solve the underlying limitation of NGS: duplex dissociation. For example, Duplex Sequencing [13], which has been the gold standard of high accuracy sequencing and utilized by other recent methods [14,15], tags double-stranded UMIs on each original duplex to trace them back after PCR and NGS. By forming a duplex consensus between reads assigned to the Watson and Crick strands of each original duplex, Duplex Sequencing achieves 1,000-fold or higher accuracy and can thus resolve true mutations within single DNA duplexes. However, recovering both strands among up to 10 billion other strands on an NGS flow cell (e.g., Illumina NovaSeq) requires 100-fold excess reads [16], which invariably diminishes the throughput of NGS and severely limits its applicability.

To date, several methods have sought to overcome the high inefficiency of Duplex Sequencing. Duplex Proximity Sequencing (Pro-Seq) [17] uses a polymer linker to link 5′-ends of original strands of a duplex, but requiring multiple PCR primers per target in the same reaction limits Pro-Seq to only small, targeted panels. Although the authors of Pro-Seq proposed an idea to address the issue, their suggestion would not be compatible with PCR which makes it impractical. Likewise, SaferSeqS also uses multiplexed PCR, limiting its applications to small, targeted panels [18]. BotSeqS [14] and NanoSeq [14,15] use dilution instead of linking to increase the chance of recovering both strands to enable Duplex Sequencing, but by doing so it only sequences 0.001% of the input DNA. CypherSeq [19] generates a circularized duplex followed by rolling circle amplification, but the lack of asymmetry between the two strands obscures whether both strands were actually sequenced. Some technologies such as o2n-seq [20] and Circle Sequencing [21] only link a single strand of a duplex and thus, lack the ability to create a duplex consensus. Despite the need for sequencing duplexes with high accuracy and throughput, there only has been methods for niche applications. It was thus reasoned that linking the information of both strands before dissociation could make NGS capable of reading single DNA duplexes with high accuracy and throughput.

The present disclosure relates to a method was that combines the massively parallel nature of NGS and the single-molecule capability of third generation sequencing to sequence both strands of each DNA duplex with single read pairs. In this hybrid approach called Concatenating Original Duplex for Error Correction (CODEC), each molecule becomes self-sufficient for forming a duplex consensus via NGS (FIG. 1A). By using the opposite strand as a template for extension instead of directly linking them, CODEC physically concatenates the sequence information of Watson and Crick strands into a single strand without forming a strong hairpin structure (FIG. 1B). Any differences between concatenated sequences would indicate either non-canonical base pairing created by nucleobase damage or an alteration confined to one strand of the original DNA duplex, or an error introduced during PCR amplification or sequencing. Because an error rate is affected by multiple factors other than a sequencing technology itself, Duplex Sequencing was performed alongside CODEC for fair comparison. CODEC was tested with both targeted and whole-genome NGS workflows to confirm that it suppressed errors as accurately as Duplex Sequencing and analyzed the mutation signatures with 100-fold and 280-fold fewer reads, respectively, thereby conferring ‘single duplex’ resolution to NGS.

The CODEC structure can be built by a streamlined workflow using a commercial ligation-based NGS preparation kit and CODEC adapter complex. First, a typical duplex adapter was replaced with the adapter complex consisting of four oligonucleotides, containing all elements required for NGS. Double-stranded segments of the adapter were rationally designed to hold the whole complex based on DNA hybridization thermodynamics (FIG. 1E) and single-stranded segments were introduced to mitigate bending stiffness of rigid double helix (FIG. 1F). After adapter ligation closes both ends of an input molecule, strand displacing extension initiates at remaining 3′-ends to elongate each strand by using the opposite strand as a template. The resulting structure is two original strands concatenated with the CODEC linker in the middle and NGS adapters on both sides. The molecular process depicted in FIG. 1B is integrated into the adapter ligation step of commercial NGS library construction kits (FIG. 1C).

To fully utilize the concatenated structure, the NGS library components were also relocated (FIG. 1D). In contrast to the conventional Illumina structure with the NGS read primer binding sites on the outer side, the read primer binding sites were moved to the CODEC linker in the middle and sequenced outward to prevent reading molecules without the linker (FIG. 2). Having the read primer binding sites at conventional locations had resulted in poor Quality Scores, which was attributed to template hopping in cluster amplification (FIG. 3A), whereas moving the read primer binding sites to the linker overcame this issue (FIG. 3B). Sample indices, which are typically located outer to the read primer binding sites and read separately from the inserts, were moved right next to the inserts. By adding the indices during adapter ligation and reading them with the inserts in a single step, CODEC suppressed index hopping even better than the gold standard of using unique dual indices [22] (0.056% vs. 0.16%). Sets of 4 sample indices were designed that collectively have all four bases at every position to ensure high base diversity for proper cluster identification, phasing correction, and chastity filtration (FIG. 4). Because indexed primers were no longer needed, Illumina P5 and P7 segments were able to be included in the adapter complex and used as universal primer binding regions.

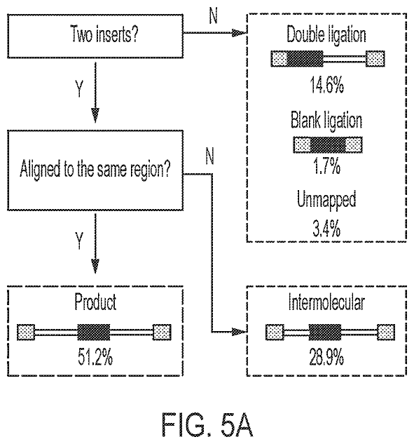

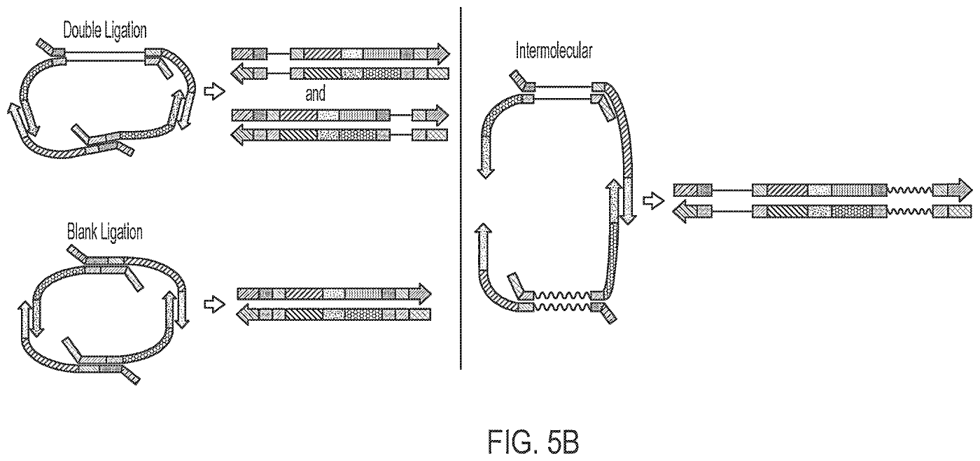

In order to confirm the feasibility of the described approach, it was first confirmed that the CODEC workflow could create the intended NGS library structure by converting fragmented human genomic DNA (gDNA) from peripheral blood mononuclear cells into a CODEC-NGS library and sequencing it. Due to the novel structure of CODEC reads, a user-friendly analysis pipeline called “CODEC suite” was created to process the data (see “Methods Related to Example 1”). It was found that more than half of the reads showed the correct structure, and almost 90% of byproducts still retained information on one side of a duplex just like standard NGS, suggesting that the byproducts may still yield useful data (FIGS. 5A-5B).

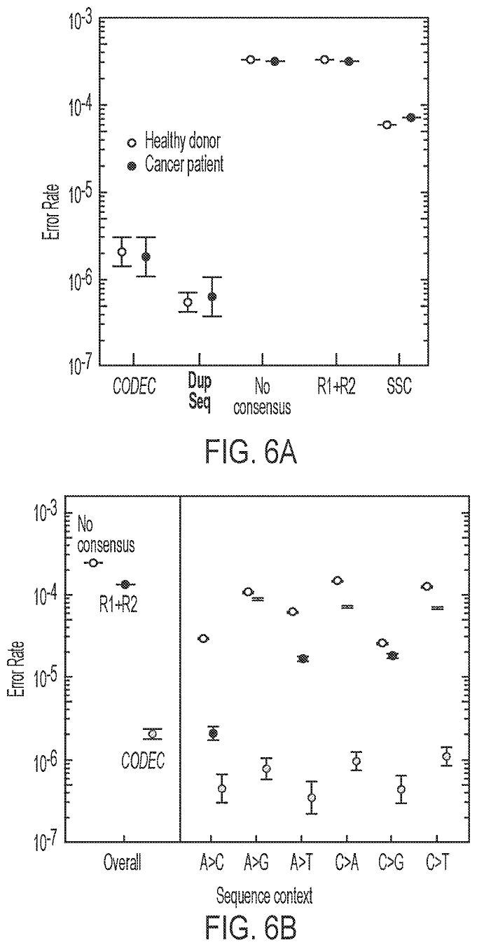

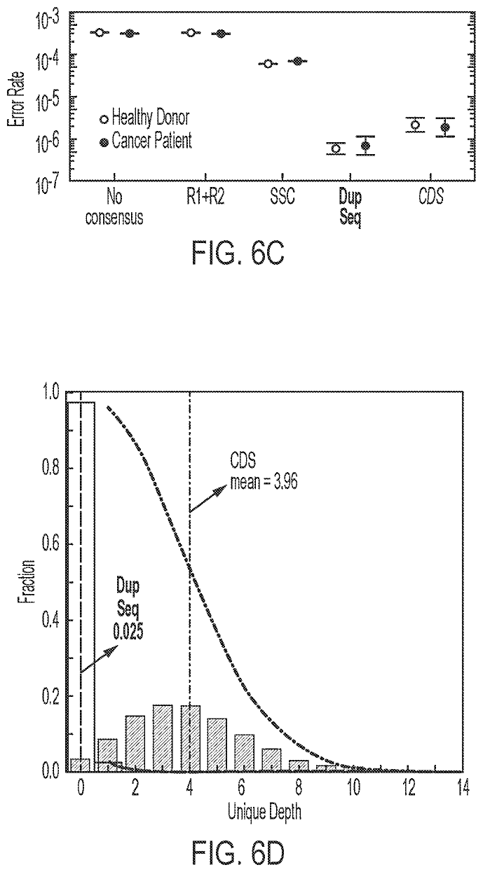

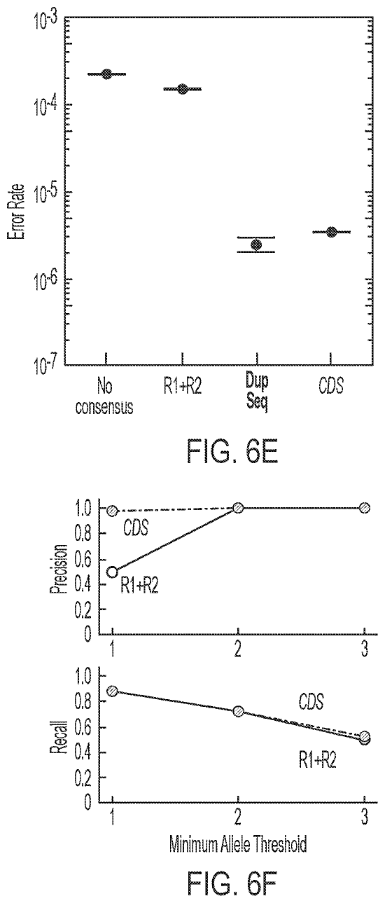

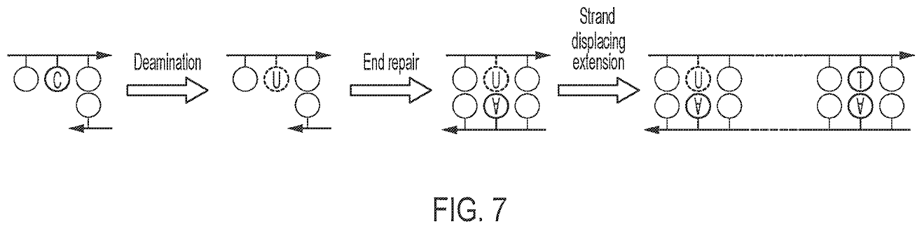

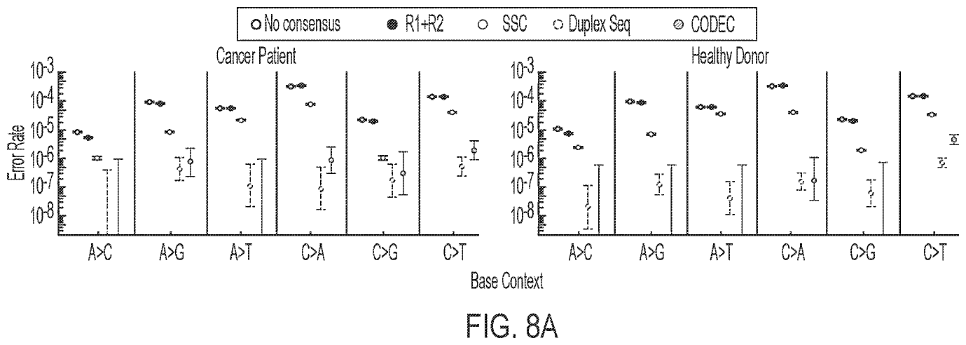

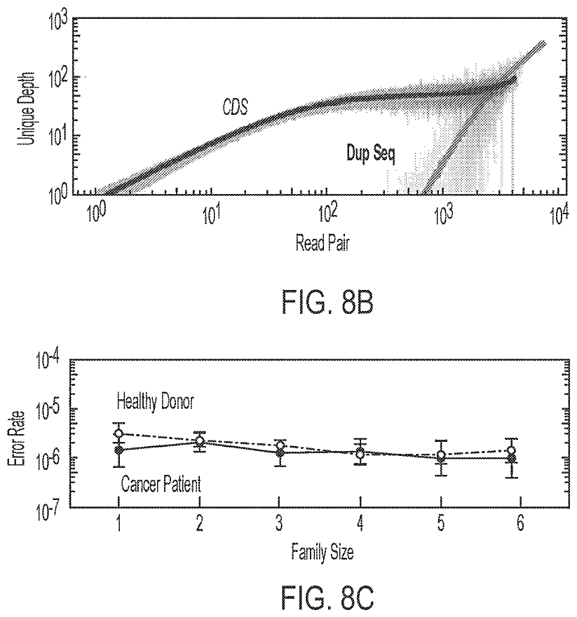

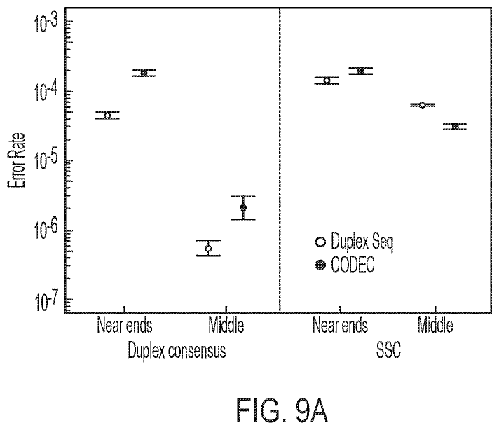

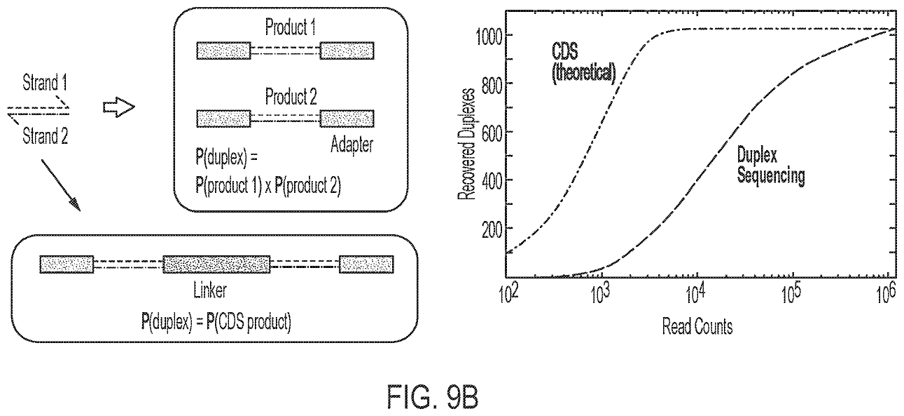

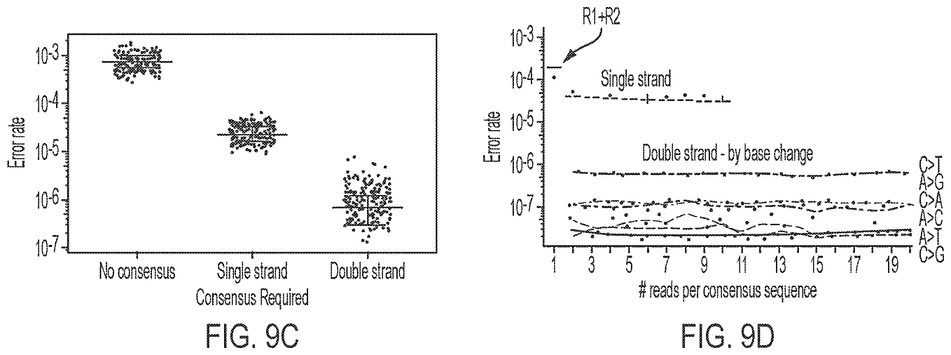

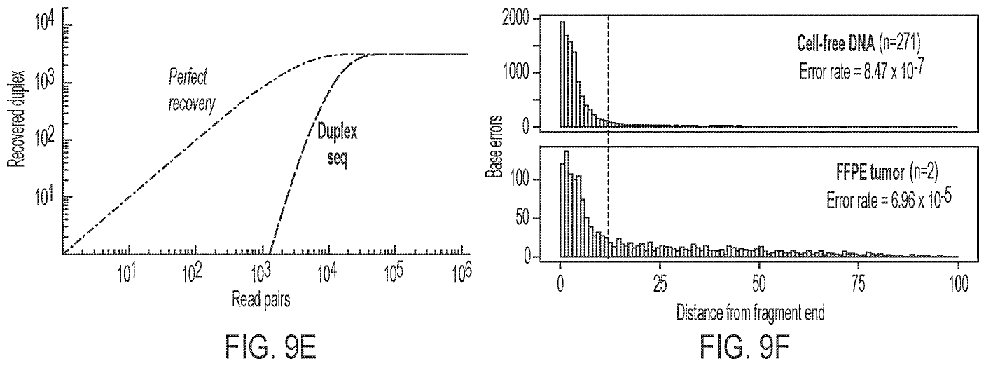

It was next explored whether the fragments with the correct CODEC structure could provide comparable error rates to Duplex Sequencing using significantly fewer reads. To assess this, a head-to-head comparison was performed. Because Duplex Sequencing requires high sequencing depth per locus, target enrichment was conducted with a pan-cancer panel on NGS libraries prepared with each method, built from 20 ng cell-free DNA (cfDNA) from a cancer patient and a healthy donor. It was found that the mean CODEC error rate of two individuals (1.9×10⁻⁶) was similar to that of Duplex Sequencing (5.9×10⁻⁷) (FIG. 6A) with no statistically significant difference in sequence contexts of errors except for C:G>T:A in a healthy donor (FIG. 7), which was believed could be resolved using an improved end-repair method [15,23](FIG. 8A). Additionally, when error rates were plotted as a function of distance from either end of a fragment, elevated error rates were seen from CODEC and Duplex Sequencing data toward the fragment ends of duplex consensus, consistent with prior reports of error propagation in end-repair [15,23] (FIG. 9A). This observation reassures that reading a single CODEC fragment is equivalent to reading two Duplex Sequencing fragments from each strand and affirms the need to trim 12 base pairs (bp) from both ends of each original DNA duplex in silico [16].

To further confirm that the error suppression potential of CODEC is uniquely enabled by reading both strands of the original DNA duplex together, as opposed to simply forming a consensus of forward and reverse reads, error rates were then compared to three additional methods from the same NGS data: no consensus, paired-end reads consensus (R1+R2, collapses read 1 and read 2), and single strand consensus (SSC, collapses reads from the same original strand). Interestingly, the error rate gap between the no consensus and R1+R2 was negligible (FIG. 6A), suggesting that many errors are physically present in NGS library molecules, and could have been introduced during library amplification, or when each library molecule undergoes cluster generation (FIG. 1A). Although SSC was more accurate than R1+R2 and the no consensus reads, without a consensus of Watson and Crick strands, its error rate was 23-fold higher than that of CODEC.

The number of reads required to uncover the same number of unique DNA duplexes were next explored. When UMIs as well as start and stop mapping positions of each molecule to collapse all reads to unique original duplexes were used, it was found that Duplex Sequencing could not start reassembling duplexes until receiving 700 reads (FIG. 8B). In contrast, CODEC started to reassemble duplexes 350-fold times earlier. The gap between required reads was maximized when recovering a smaller number of duplexes, suggesting that CODEC could be uniquely capable of sequencing broad genomic regions with shallow depth. Notably, even a single paired-end read of CODEC was highly accurate (FIG. 6A), as each CODEC read is self-sufficient to form a duplex consensus. These results suggest that CODEC confers the accuracy of duplex sequencing from single paired-end reads and thus sequences more DNA duplexes using substantially fewer reads.

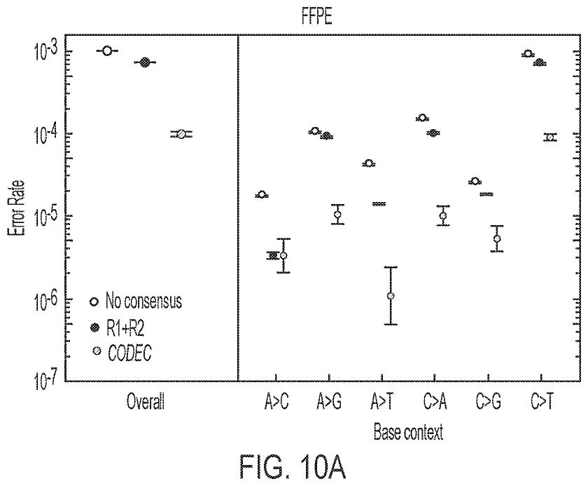

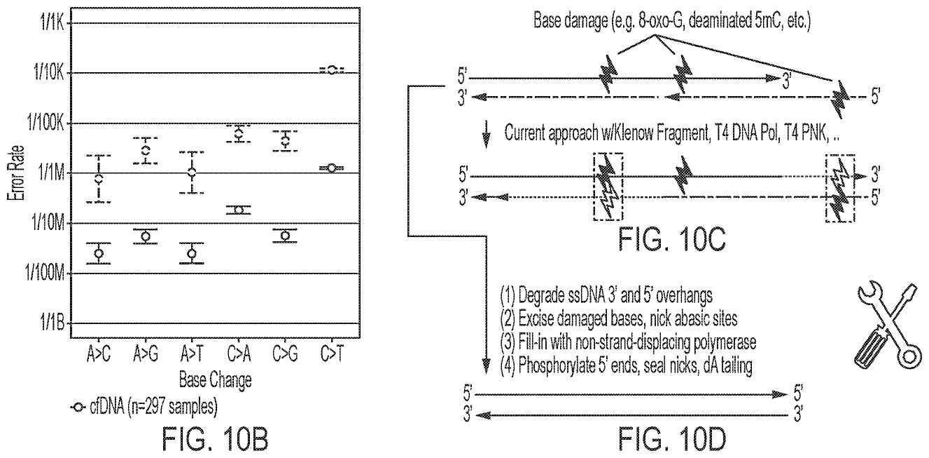

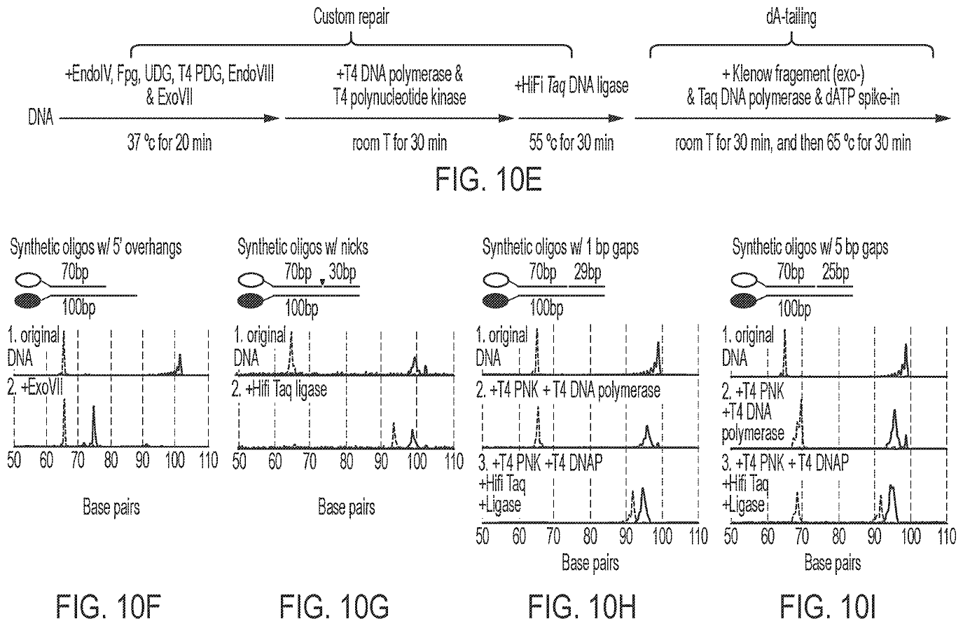

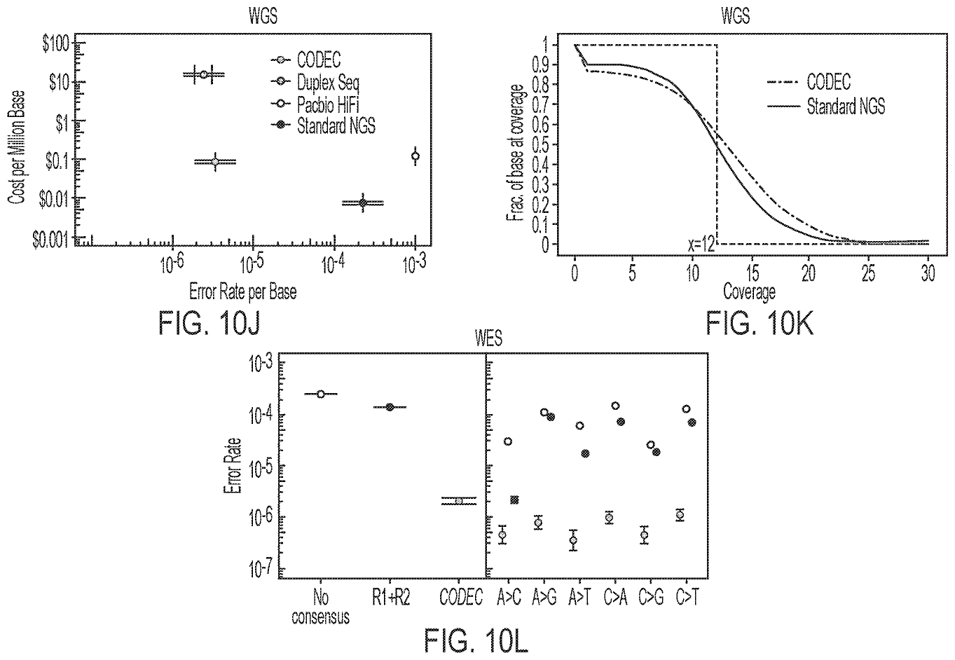

Next it was sought to determine whether CODEC could enable human whole-exome and whole-genome ‘duplex’ sequencing, which would otherwise be impractical due to high cost. To assess this, CODEC whole-exome sequencing (WES) was applied to human gDNA, whose samples had been tested previously [16]. It was found that CODEC reduced the sequencing error rates of both samples, with 100-fold improvement for gDNA (FIG. 6A). Analyzing the sequence context of the errors revealed that CODEC improved accuracy across all types of SNV, suggesting that the capability of CODEC to suppress errors is not limited to specific contexts. Of note, there were more C>T errors in FFPE sample (FIG. 10A) due to deamination artifacts [24], which could be resolved with improved end-repair methods [15, 23].

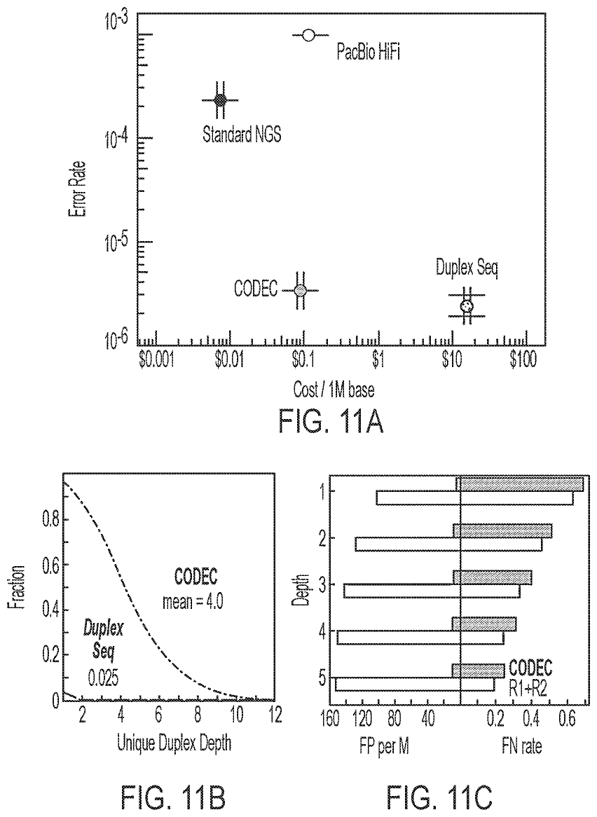

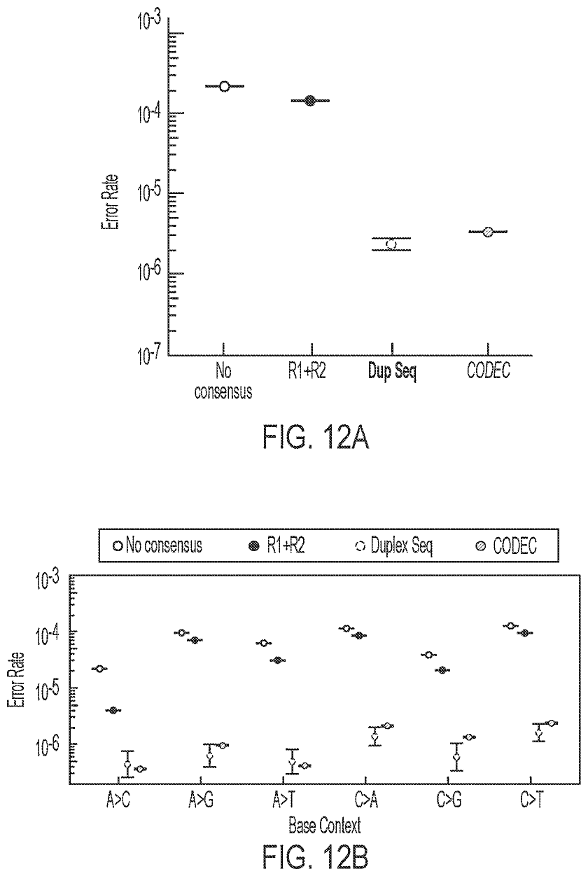

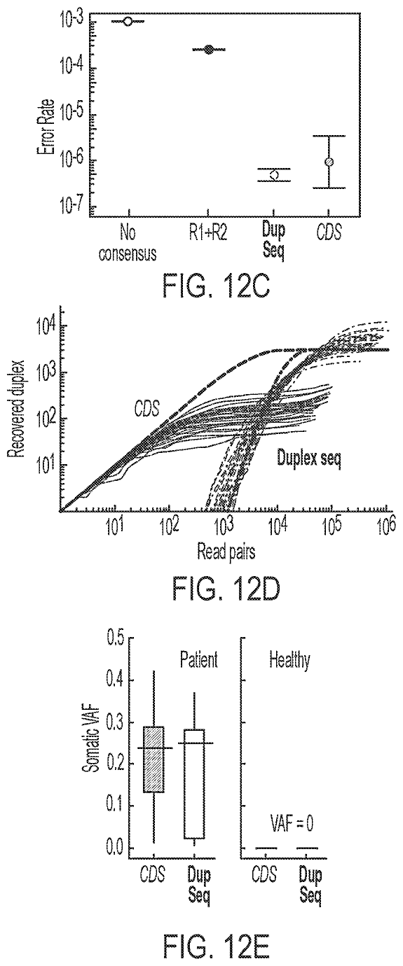

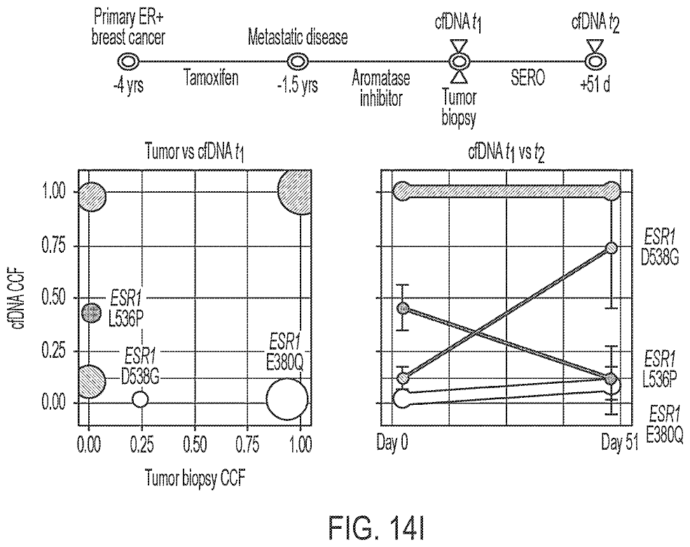

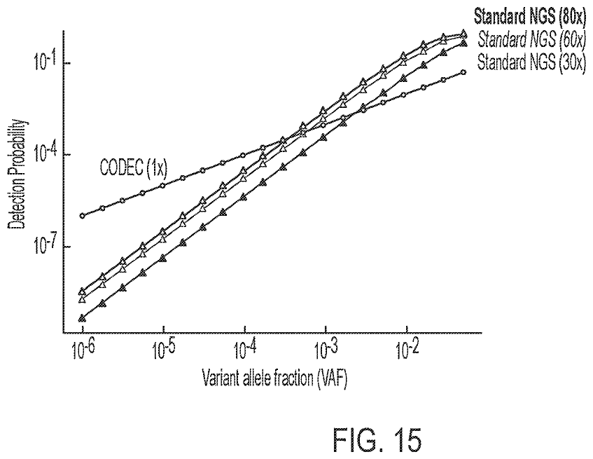

Next, CODEC and Duplex Sequencing were applied to WGS of the pilot genome NA12878 of the Genome in a Bottle Consortium (GIAB) [25]. The same amount of sequencing was assigned to each method for a fair comparison although Duplex Sequencing could not recover many unique duplexes. In cost-benefit analysis, the error rates of both Duplex Sequencing (2.38×10⁻⁶) and CODEC (3.37×10⁻⁶) were much lower than that of standard NGS (2.2×10⁻⁴) (FIG. 11A), which showed similar results to R1+R2 (FIG. 12A). This confirms that CODEC is as accurate as Duplex Sequencing under the same conditions. In addition, the error rates of each sequence context showed that CODEC has a similar error profile to Duplex Sequencing (FIG. 15). In terms of the sequencing cost, Duplex Sequencing is 100-1000 times more expensive than the other methods, limiting its applicability to targeted panels.

Depth of coverage analysis for WGS further demonstrated that CODEC achieved 160-fold greater unique duplex depth than Duplex Sequencing. On the GIAB v3.3.2 hg19 high confidence genomic region (2.6B bases), CODEC had a mean unique duplex depth of 4.0, whereas Duplex Sequencing had only 0.025 mean depth even with 35% more raw read output, because most reads did not find their matching strand of the original duplex (FIG. 11B). Thus, it was concluded that Duplex Sequencing is not practical for WGS and Duplex Sequencing WGS data were treated as standard WGS data without generating duplex consensus after this point. CODEC, on the other hand, breaks a traditional trade-off between accuracy and cost which has been a dilemma of the existing methods thanks to the strength of resolving single duplexes.

CODEC pushes the frontiers in secondary analysis applications. Achieving the error rate of Duplex Sequencing in WGS/WES gives CODEC the ability to push the limits of many secondary analysis applications. One such application is benchmarking the whole genome small germline variant calling (SNV+indel). To test the potential of CODEC at low coverage as implied in FIG. 8B, CODEC data of the aforementioned NA12878 sample was compared against R1+R2 at coverages ranging from 1× to 5×, while acknowledging that state-of-the-art germline calling usually requires 30× depth. GATK4 [26] was used for variant calling and followed by the GIAB best practice for benchmarking small germline variants. CODEC showed 90% fewer false positives (FP) than standard WGS with R1+R2 at a cost of 5% higher false negatives (FN) across all downsampled depths (FIG. 11C, Table 1).

TABLE 1
Evaluation of SNP + small indel calls between CODEC WGS and standard WGS. Table was generated by Vcfeval.

| | True- | | | | | | | | | | | |
|---|---|---|---|---|---|---|---|---|---|---|---|---|
| | pos- | True- | False- | False- | | Sensi- | F- | | ds_ | | | |
| Method | baseline | pos-call | pos | neg | Precision | tivity | measure | type | frac | FPPM | FNR | depth |
| CODEC | 1149572 | 1149620 | 11542 | 2541289 | 0.9901 | 0.3115 | 0.4739 | na12878_cds | 0.1 | 4.482217885 | 0.688526593 | 1 |
| | 1786822 | 1786884 | 17480 | 1904039 | 0.9903 | 0.4841 | 0.6503 | na12878_cds | 0.2 | 6.788179573 | 0.515870691 | 2 |
| | 2222868 | 2222945 | 21154 | 1467993 | 0.9906 | 0.6023 | 0.7491 | na12878_cds | 0.3 | 8.21493997 | 0.397728978 | 3 |
| | 2544500 | 2544593 | 23624 | 1146361 | 0.9908 | 0.6894 | 0.8131 | na12878_cds | 0.4 | 9.174139258 | 0.31058664 | 4 |
| | 2782660 | 2782755 | 25191 | 908201 | 0.991 | 0.7539 | 0.8564 | na12878_cds | 0.5 | 9.782667713 | 0.246061183 | 5 |
| | 2959035 | 2959132 | 25577 | 731826 | 0.9914 | 0.8017 | 0.8865 | na12878_cds | 0.6 | 9.932566873 | 0.198275353 | 6 |
| | 3089997 | 3090099 | 25548 | 600864 | 0.9918 | 0.8372 | 0.908 | na12878_cds | 0.7 | 9.921305019 | 0.162793287 | 7 |
| | 3186894 | 3186995 | 25231 | 503967 | 0.9921 | 0.8635 | 0.9233 | na12878_cds | 0.8 | 9.798201304 | 0.136540826 | 8 |
| | 3259747 | 3259845 | 24540 | 431114 | 0.9925 | 0.8832 | 0.9347 | na12878_cds | 0.9 | 9.529858508 | 0.116802706 | 9 |
| | 3314661 | 3314761 | 23829 | 376200 | 0.9929 | 0.8981 | 0.9431 | na12878_cds | 1 | 9.253748915 | 0.101924675 | 10 |
| Standard | 1362228 | 1362280 | 263618 | 2328633 | 0.8379 | 0.3691 | 0.5124 | na12878_r1r2 | 0.077 | 102.3733594 | 0.630909751 | 1 |
| NGS | 2001169 | 2001242 | 332255 | 1689692 | 0.8576 | 0.5422 | 0.6644 | na12878_r1r2 | 0.15 | 129.0278378 | 0.457795236 | 2 |
| | 2453841 | 2453932 | 366744 | 1237020 | 0.87 | 0.6648 | 0.7537 | na12878_r1r2 | 0.23 | 142.4212889 | 0.335149306 | 3 |
| | 2778411 | 2778517 | 387976 | 912450 | 0.8775 | 0.7528 | 0.8104 | na12878_r1r2 | 0.31 | 150.6665193 | 0.247211639 | 4 |
| | 2990581 | 2990709 | 392938 | 700280 | 0.8839 | 0.8103 | 0.8455 | na12878_r1r2 | 0.38 | 152.5934614 | 0.189726927 | 5 |
| | 3169483 | 3169605 | 380937 | 521378 | 0.8927 | 0.8587 | 0.8754 | na12878_r1r2 | 0.46 | 147.9329955 | 0.141257221 | 6 |
| | 3296709 | 3296823 | 354625 | 394152 | 0.9029 | 0.8932 | 0.898 | na12878_r1r2 | 0.54 | 137.7149989 | 0.106788044 | 7 |
| | 3386823 | 3386934 | 320037 | 304038 | 0.9137 | 0.9176 | 0.9156 | na12878_r1r2 | 0.62 | 124.2831022 | 0.082373424 | 8 |
| | 3443475 | 3443589 | 289759 | 247386 | 0.9224 | 0.933 | 0.9276 | na12878_r1r2 | 0.69 | 112.5249499 | 0.067024567 | 9 |
| | 3491018 | 3491139 | 258701 | 199843 | 0.931 | 0.9459 | 0.9384 | na12878_r1r2 | 0.77 | 100.4638927 | 0.054143586 | 10 |

ds_frac: downsample fraction.
FFPM: False positive per million bases.
FNR: False negative ratio

By downsampling NGS data, it was also observed how FP and FN are affected by the depth. The lower level of FP in CODEC was the expected result, considering its lower error rate. Its FN levels were slightly higher than that of standard WGS, probably because the lower library conversion efficiency resulted in higher duplication rate, but the difference between FN rates of CODEC and standard WGS became smaller as the coverage decreased. Meanwhile, the advantage of having low FP became more significant at the lower coverage, implying that applications with shallow depth could benefit more from using CODEC.

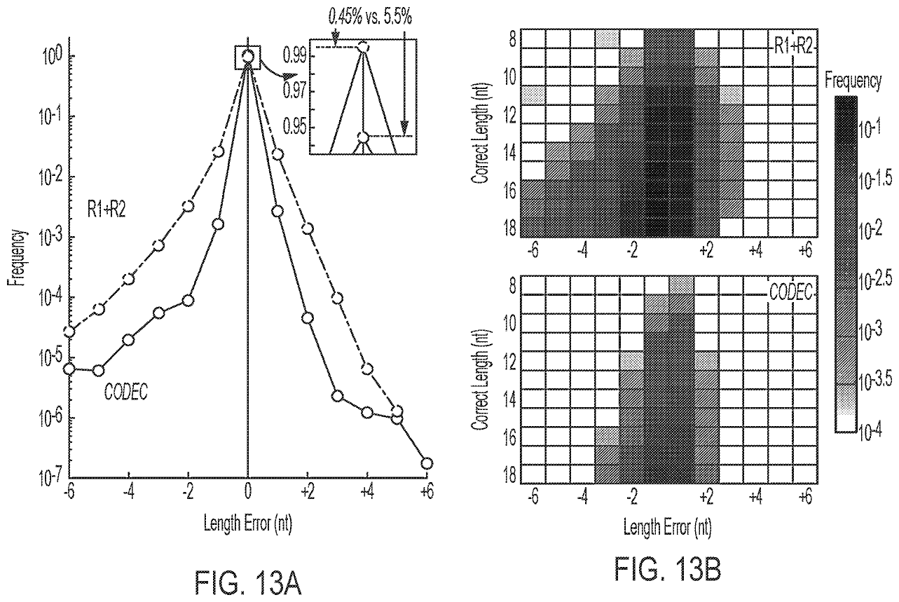

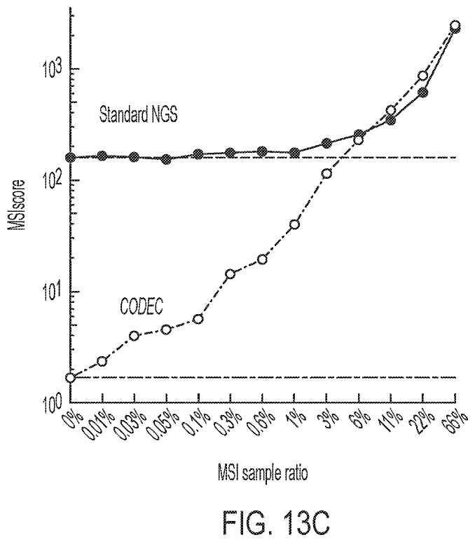

Considering CODEC's performance for indel detection at low coverage, it was thought that CODEC could improve the sequencing accuracy of microsatellites (MS), which are well-known mutation hot spots. Indeed, when the reference sequences of the mononucleotide MS in NA12878 were compared between CODEC and standard NGS results, CODEC showed lower frequencies of both insertion and deletion errors (FIG. 13A). The ratio of CODEC reads with incorrect MS lengths was 0.45%, which was 12 times lower than that of standard WGS. Such lower frequencies were consistently observed across mononucleotide MS of varied lengths from 8 to 18 nucleotides (FIG. 13B), especially for deletion at longer MS. These findings imply that CODEC could be used to read the repeat numbers/copy numbers of MS sites for detecting microsatellite instability (MSI), which has been shown to be a predictive marker of response to cancer immunotherapy but remains challenging to detect at low frequency such as from liquid biopsy samples [27]. Thus, an MSI sample was sequenced and its matching normal sample to test if CODEC improves the existing MSI detection limit (0.1%). When detecting MSI from an in silico dilution series, MSMuTect analysis [28] reduced the detection limit of standard NGS to 0.1%, whereas that of CODEC data was 0.01% (FIG. 13C). The improvements in the secondary applications highlight what CODEC could enable by sequencing a single duplex within each NGS cluster.

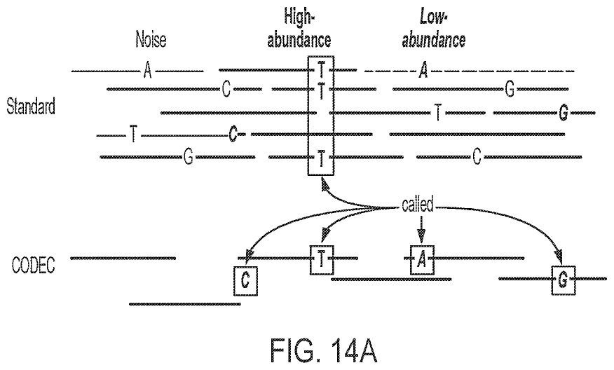

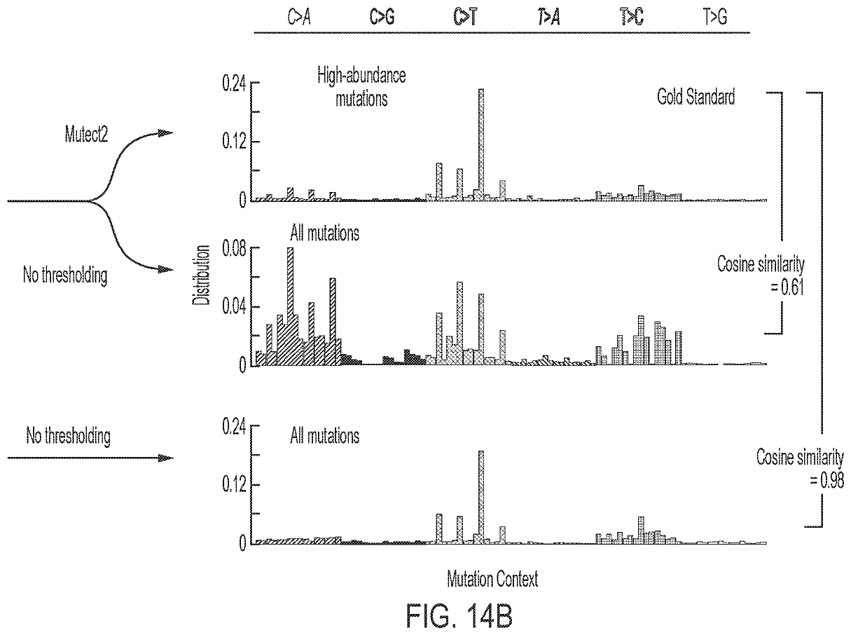

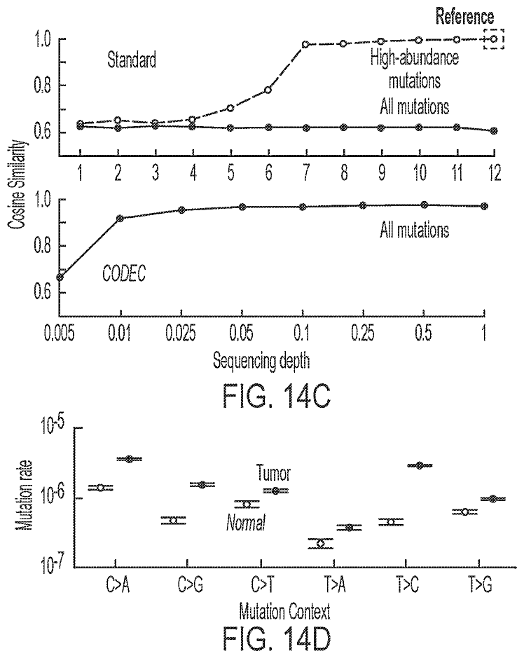

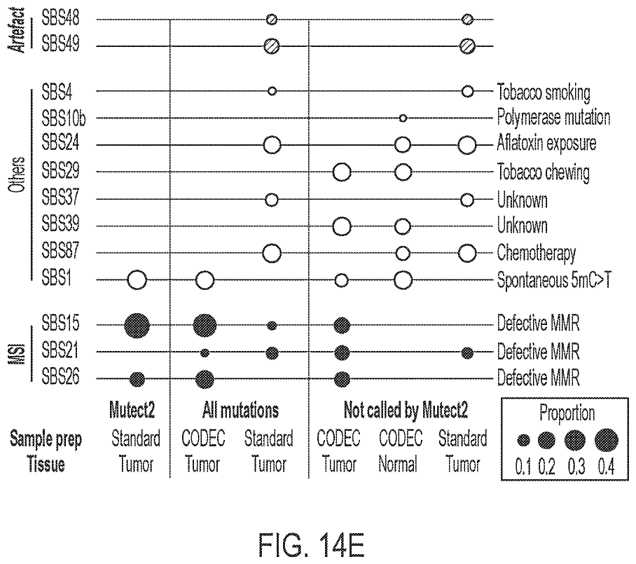

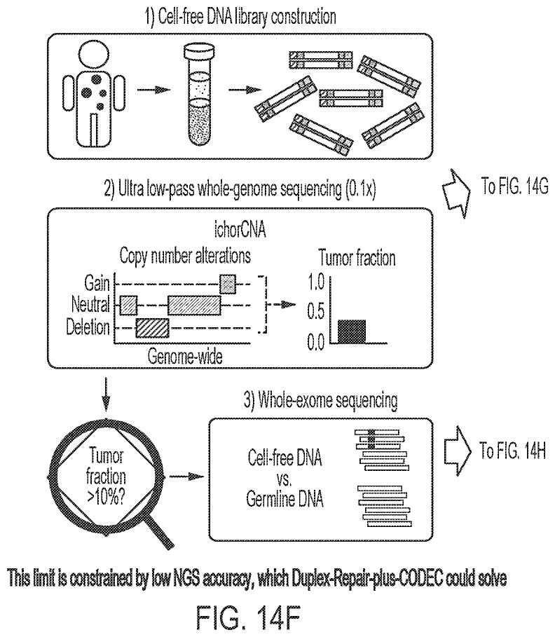

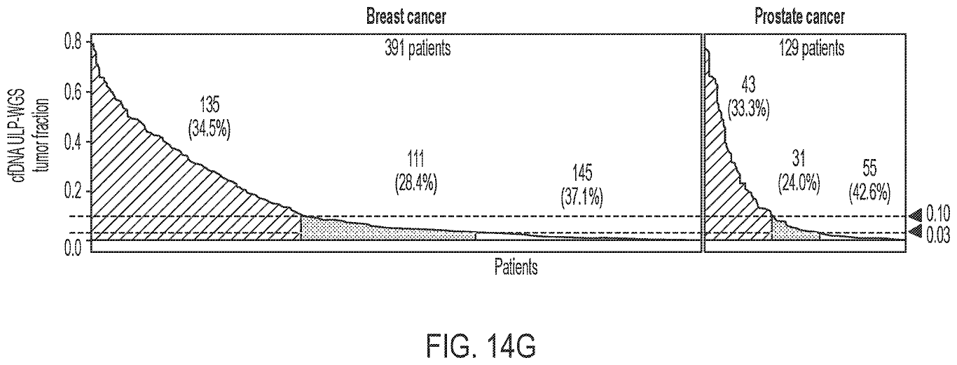

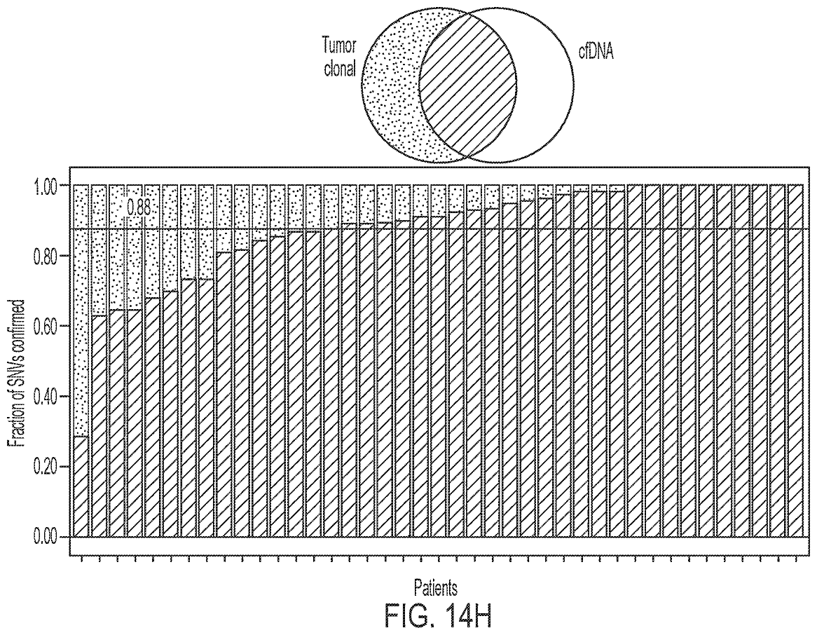

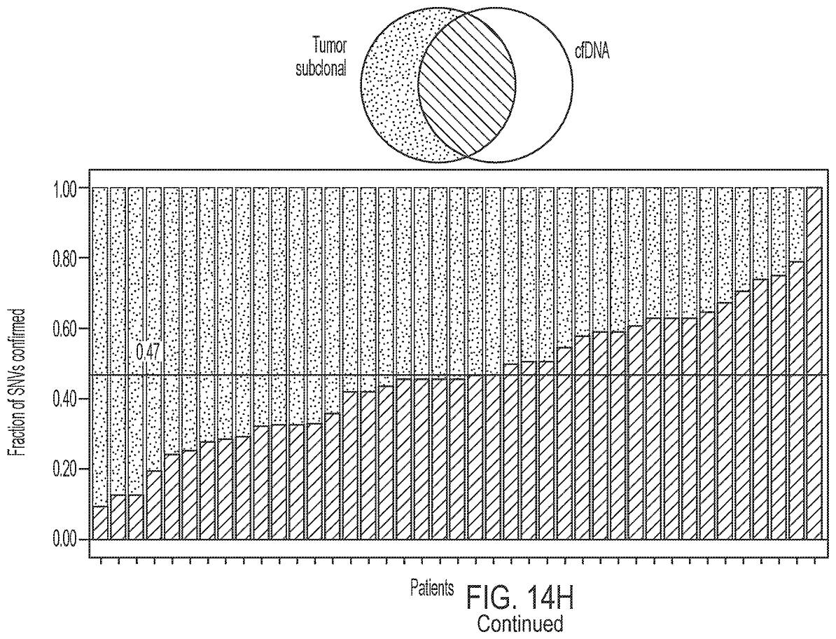

CODEC offers single molecule mutation signatures. To explore the potential of detecting somatic mutations with low-depth CODEC WGS, trinucleotide context of mutations were compared in MSI sample detected by CODEC (1× coverage) and standard NGS (12× coverage) paired with a variant caller, Mutect2 [29]. The main difference is that variant callers discard low-abundance mutations due to high background noise while CODEC can accept both high- and low-abundance mutations (FIG. 14A). For example, accepting all single base substitutions (SBS) of standard NGS without a statistical thresholding significantly changed their context compared to that of high-abundance mutations (cosine similarity=0.61) (FIG. 14B), whereas accepting all mutations from CODEC resulted in the same context (cosine similarity=0.98). This implies that standard NGS cannot distinguish low-abundance mutations from random errors while lower error rate of CODEC enables calling low-abundance mutations without multiple reads (FIG. 15). The same trend was consistently observed at even lower CODEC sequencing depth. When standard NGS and CODEC data was downsampled, the context of high-abundance mutations below 7× depth started to deviate from that of 12× depth, which were used as the reference to calculate cosine similarities (FIG. 14C). Accepting all mutations of standard NGS consistently failed to pick up the same mutation context at all sequencing depths. In contrast, CODEC successfully yielded the same mutation context even at 0.025× (cosine similarity=0.95), reducing the sequencing depth required by 280-fold to call low-abundance mutations.

After confirming the capability of CODEC to detect rare mutations, it was next sought to determine if mutations detected exclusively by CODEC are true somatic mutations. It was hypothesized that tumor samples, which have subclonal somatic mutations, would show more low-abundance mutations exclusive to CODEC than normal samples. In fact, the rate of such mutations was 2.7 times higher in a tumor sample (FIG. 14D), and the difference went up to 6.4-fold in T>C substitution, which is enriched in some mutational signatures associated with MMR deficiency [30]. To further analyze the exclusive mutations, Catalogue Of Somatic Mutations In Cancer (COSMIC) mutational signatures were then extracted from different sets of mutations (FIG. 14E). CODEC detected not only the signatures in Mutect2 data but also one more MSI signature (SBS21), and utilizing all mutations from standard NGS canceled most of the MSI signatures. Of note, SBS1 signature comes from deamination of 5-methylcytosine to thymine which is observed in both tumor and normal cells. Signatures of mutations detected by CODEC but discarded by Mutect2 resembled those of all mutations from CODEC, suggesting that they were low-abundance somatic mutations as well. Interestingly, SBS29, one of two new signatures missed by Mutect2, is related to tobacco chewing that may have affected tumor and normal tissues, both from the colon of the patient. It was also confirmed that normal tissue showed none of MSI signatures in CODEC data and that mutations from standard NGS discarded by Mutect2 still showed scattered signatures. Thus, single duplex resolution of CODEC enabled detecting mutational signatures better than standard NGS paired with a variant caller with significantly less sequencing.

By physically linking both strands of each DNA duplex, CODEC enables each NGS cluster to have single duplex resolution like third generation sequencers. Unlike Duplex Sequencing which requires dissociating duplexes and recovering them back to form a duplex consensus, CODEC distinguishes real mutations from errors with similarly high accuracy but with 100-fold fewer reads. This approach was first shown using cfDNA enriched by a pan-cancer panel, followed by testing its consistency across other major NGS workflows (e.g., WES and WGS). To present more applications of CODEC, it was also shown that it suppressed FP especially at shallow sequencing depth, reduced indel errors at MS sites, and detected mutational signatures from a cancer patient at ultra-low sequencing depth.

In a head-to-head comparison, it was shown that CODEC is as accurate as Duplex Sequencing but with a much lower sequencing requirement, which has been a major limitation of Duplex Sequencing. Because an error rate is affected by multiple factors other than a sequencing technology itself, any direct comparison requires everything else to be the same. The same experimental and computational protocols were used whenever applicable, including input samples and mass, reagents, target regions, definition of an error, and analysis pipelines for precise comparison.

The CODEC adapter complex is attached through two consecutive ligations: a bimolecular ligation followed by a unimolecular ligation. Unlike typical bimolecular adapter ligation where increasing adapter concentration also increases conversion efficiency, unimolecular ligation could be less favorable when the adapter concentration is too high. Consequently, the current version of CODEC adapter complex needs balancing between two ligations.

Although conventional end-repair/dA-tailing of a commercial kit was used throughout this work, the accuracy can be further improved if a new end-repair method is adopted before CODEC. Recent studies [15,23] have reported that base damage on overhangs and single-stranded breaks of original DNA duplexes can lead errors on one strand to be copied to both strands. It was also indirectly observed in this work that error rates were generally higher toward the ends of DNA fragments (FIG. 9A). While such errors appear on duplex consensus and result in false mutations, new end-repair methods prevent the error propagation, and it is believed that even higher accuracy will be attainable when CODEC is combined with new end-repair methods.

Reading a single CODEC fragment is equivalent to reading both strands of an original duplex, which eliminates the need to read the same locus multiple times. The low error rate of CODEC at 1× read depth opens possibilities for various applications across fields from diagnostics to bioinformatics. One example is discovering rare somatic mutations with a limited number of reads, which has a higher chance of finding a true mutation when the error rate gets lower [32]. Another example is shotgun metagenomic sequencing for microbiome analysis, where suppressing false SNVs with CODEC would prevent incorrect taxonomic classifications and inaccurate evaluation of microbial diversity [33]. In de novo assembly, lower error rates contribute to more contiguous assembly in de Bruijn graph paradigm and faster process in overlap-layout-consensus paradigm [34].

In summary, CODEC transforms standard NGS instruments into massively parallel single duplex sequencers by concatenating both strands of each original DNA duplex. This strategy enables SNV and indel detection as accurate as Duplex Sequencing with significantly fewer reads and cancer signature detection with sequencing depth as low as 0.025×. Moreover, the applicability of CODEC ranging from a targeted sequencing to WGS sets it apart from other high-accuracy NGS methods. Thus, it is believed that CODEC could be broadly enabling for many important biomedical applications such as detecting early-stage cancer or minimal residual disease from liquid biopsies, clinically actionable mutations from liquid or tumor biopsies, clonal hematopoiesis of in determinate potential (CHIP) from blood samples, somatic mosaicism in normal tissue samples, and beyond.

### Methods Related to Example 1

### DNA Samples and Oligonucleotides.

Cell-free DNA of patient 315 from cohort 05-246 and both FFPE and gDNA of patient 95 from cohort 05-055 were from another study [16]. MSI DNA of patient 19 was also from another study [27]. NA12878 was purchased from Coriell. All samples were stored in low TE buffer (10 mM Tris-HCl, 0.1 mM EDTA, pH 8) and were fragmented by Covaris ultrasonicator to have a mean size of 150 bp except cfDNA. All oligonucleotides for CODEC were synthesized by Integrated DNA Technologies (IDT) and went through PAGE purification (Table 2). The adapter for Duplex Sequencing was custom-ordered for the Broad Institute by IDT.

TABLE 2
Sequences of oligonucleotides.

| | | SEQ ID |
|---|---|---|
| CODEC Adapter | | NO: |
| LD4-adap5-1 | AATGATACGGCGACCACCGAGATCTACACCTTGAACGGACTGTCCAC*T | 1 |
| LD4-adap5-2 | AATGATACGGCGACCACCGAGATCTACACGAGCCTACTCAGTCAACG*T | 2 |
| LD4-adap5-3 | AATGATACGGCGACCACCGAGATCTACACGCTTGTAAGGCAGGTTAG*T | 3 |
| LD4-adap5-4 | AATGATACGGCGACCACCGAGATCTACACCAAGCGTCTTACATGGTC*T | 4 |
| LD4-adap7-1 | CAAGCAGAAGACGGCATACGAGATCACCGAGCGTTAGACTAC*T | 5 |
| LD4-adap7-2 | CAAGCAGAAGACGGCATACGAGATGTGTCGAACACTTGACGG*T | 6 |
| LD4-adap7-3 | CAAGCAGAAGACGGCATACGAGATCTGATCTTCAGCTGACTG*T | 7 |
| LD4-adap7-4 | CAAGCAGAAGACGGCATACGAGATGAATCTGAGGCACTGTAC*T | 8 |
| LD4-brid5-1 | P-GTGGACAGTCCGTTCAAGNNNAGATCGGAAGAGCGTCGTGTAGGGAA | 9 |
| | AGAGTGTTTACATAGTTATCCGCTAGACTCTGACGTGTTGATCCTCGAA | |
| | GC | |
| LD4-brid5-2 | P-CGTTGACTGAGTAGGCTCNNNAGATCGGAAGAGCGTCGTGTAGGGAA | 10 |
| | AGAGTGTTTACATAGTTATCCGCTAGACTCTGACGTGTTGATCCTCGAA | |
| | GC | |
| LD4-brid5-3 | P-CTAACCTGCCTTACAAGCTNNNAGATCGGAAGAGCGTCGTGTAGGGA | 11 |
| | AAGAGTGTTTACATAGTTATCCGCTAGACTCTGACGTGTTGATCCTCGA | |
| | AGC | |
| LD4-brid5-4 | P-GACCATGTAAGACGCTTGANNNAGATCGGAAGAGCGTCGTGTAGGGA | 12 |
| | AAGAGTGTTTACATAGTTATCCGCTAGACTCTGACGTGTTGATCCTCGA | |
| | AGC | |
| LD4-brid7-1 | P-GTAGTCTAACGCTCGGTGNNNAGATCGGAAGAGCACACGTCTGAACT | 13 |
| | CCAGTCACCAATCTATAAGTTGCTTCGAGGATCAACACGTCAGAGTCTA | |
| | GC | |
| LD4-brid7-2 | P-CCGTCAAGTGTTCGACACNNNAGATCGGAAGAGCACACGTCTGAACT | 14 |
| | CCAGTCACCAATCTATAAGTTGCTTCGAGGATCAACACGTCAGAGTCTA | |
| | GC | |
| LD4-brid7-3 | P-CAGTCAGCTGAAGATCAGTNNNAGATCGGAAGAGCACACGTCTGAAC | 15 |
| | TCCAGTCACCAATCTATAAGTTGCTTCGAGGATCAACACGTCAGAGTCT | |
| | AGC | |
| LD4-brid7-4 | P-GTACAGTGCCTCAGATTCANNNAGATCGGAAGAGCACACGTCTGAAC | 16 |
| | TCCAGTCACCAATCTATAAGTTGCTTCGAGGATCAACACGTCAGAGTCT | |
| | AGC | |

CODEC Blocker for Hybridization Capture

| | | |
|---|---|---|
| LD4-HybBlk1 | AGATCGGAAGAGCGTCGTGTAGGGAAAGAGTGTTTACATAGTTATCCGC | 17 |
| | TAGACTCTGACGT-3C | |
| LD4-HybBlk2 | AGATCGGAAGAGCACACGTCTGAACTCCAGTCACCAATCTATAAGTTGC | 18 |
| | TTCGAGGATCAAC-3C | |
| Key | Illumina P5 | |
| | Illumina P7 | |
| | Sample index | |
| | UMI | |
| | Read primer binding regions | |
| | Insert | |

“*” between nucleotides indicates phosphorothioate backbone modification.
“P-” indicates 5′-phosphorylation.
“-3C” indicates C3 spacer.

### Preparation of CODEC Adapter

The CODEC adapter complex was prepared by diluting four 100 μM oligonucleotides to μM with low TE buffer and 100 mM NaCl, followed by heating at 85° C. for 3 minutes, cooling with −1° C./min to 20° C., and incubating at room temperature for 12 hours. Mastercycler X50 (Eppendorf) and MAXYMum Recovery PCR tubes (Axygen) were used for the annealing. The annealed adapter complex was kept at −20° C. for future use. NEBNext Ultra II DNA Library Prep Kit for Illumina (New England Biolabs, NEB) was used and the manufacturer's manual was followed with several exceptions:
1. ligation time was increased to 1 hour, 5 μM adapter complex was diluted with adapter dilution buffer (10 mM Tris-HCl, 1 mM EDTA, 10 mM NaCl, pH 8) to 500 nM before use and replaced NEB adapter,
2. 3 μL of 50-deadenylase (NEB) were added to ligation reaction,
3. strand displacing extension (sample 40 μL, 10× buffer 10 μL, 0.2 mM dNTP, polymerase 1 μL, H2O up to 100 μL) was performed with phi29 DNA polymerase (New England Biolabs) at 30° C. for 20 minutes, followed by standard AMPure XP (Beckman Coulter) clean up with 0.75× volume ratio,
4. KAPA HiFi HotStart ReadyMix and xGen Library Amplification Primer Mix (IDT) were used for PCR by following the manufacturer's manuals with 2 minutes of extension,
5. and AMPure XP clean up with 0.75× volume ratio was performed twice after the PCR.

Libraries for standard NGS and Duplex Sequencing were prepared as described elsewhere [16]. All Library preparations were performed on twin.tec PCR Plates LoBind 250 μL (Eppendorf). Library quantitation was performed with Qubit dsDNA HS kit (Invitrogen) paired with Bioanalyzer DNA High Sensitivity chips (Agilent).

### Enrichment.

Both pan-cancer and WES enrichment was performed with xGen Hybridization and Wash kits and xGen Blocking Oligos (IDT), following the manufacturer's manual. For capture probes, xGen Pan-cancer Panel (IDT, 800 kb) and custom WES panel for the Broad Institute by Twist Bioscience were used.

### Sequencing.

Standard NGS and Duplex Sequencing were performed with Illumina HiSeq 2500 Rapid Run (300 cycles) for a pan-cancer panel and WGS. CODEC was performed with Illumina HiSeq 2500 Rapid Run (500 cycles) for a pan-cancer panel and WGS, and NovaSeq SP (500 cycles) for WGS and WES. The extra cycles were used to confirm the CODEC structure.

### CODEC Data Processing.

Due to the unique CODEC read structure, CODECsuite (available at github.com/broadinstitute/CODECsuite) (the entire contents of which are incorporated herein by reference) was developed to process CODEC data. CODECsuite is written in C++ 14 and python3.7 and snakemake6.0.3 was used as the workflow management system. CODECsuite consists of 4 major steps: demultiplexing, adapter trimming, consensus calling and computing accuracy. The first 3 steps are specific to CODEC data. The workflow also involves other standard tools such as BWA, Fgbio and GATK Illumina bcl2fastq was used to generate fastq files (with -R -o, no -sample-sheet because CODECsuite will demultiplex), but is not included in the suite. To speed up the data processing, splitting the fastq files in batches and processing them in parallel is recommended. In this Example, using 40 batches, the preprocessing (demultiplexing and adapter trimming) of 800M NovaSeq reads took just a few hours in a HPC environment where each batch was executed using a single CPU and 8G RAM. After demultiplexing and adapter removal, the raw reads were mapped using BWA(0.7.17-r1188) against human reference hgl9. Fgbio (github.com/fulcrumgenomics/fgbio) was then used to collapse the PCR duplicates and to form essentially single-strand consensus (SSC) reads. These SSC reads were then mapped to the reference genome using BWA again. Next, the duplex consensus reads between R1 and R2 were generated from the SSC alignments. A consensus base was filtered if any of the bases from R1 or R2 has base quality less than 30. The duplex consensus reads were aligned to the reference genome using BWA and the subsequent alignments were indel realigned using GATK3 (hub.docker.com/r/broadinstitute/gatk3).

### Demultiplexing.

CODEC sequencing reads start with Unique Molecular Identifier (UMI) sequences: NNN or NNNA or NNNT (NNN is a random 3-mer), and follow by an 18 bp sample barcode and then a T base (FIGS. 11A-11C). To demultiplex, CODECSuite extracts the barcode (4th-21st bases from the 50-end) and uses smith-waterman (SW) algorithm [1] for sample indices (SID) assignments. If the extracted barcode is within x edit distance (default 3) away from one and only one sample index, it is declared as a match. Then, a read pair is successfully demultiplexed if and only if the two extracted barcodes (one from each end of the read pair) both match the expected SID (P5 and P7). Only successfully demultiplexed reads are used for subsequent steps and the expected SID are stored in the read names for the subsequent adapter trimming step. Besides, when the two barcodes from a read pair match a chimeric sample index combination, CODECsuite also checks index hopping by aligning the two inserts and flags them as hopping reads if they overlap. Otherwise, the mixed indices are most likely a result of intermolecular byproduct.

### Adapter Trimming and Byproducts Cleaning.

The demultiplexing step adds SID to the read name but does not alter the read sequencing. The adapter trimming step removes the adapter sequences from the read and output as uBAM (unmapped BAM format). The first 3 bases of R1 and R2 are cut and hyphenated and added to the ‘RX’ tag in the bam record. Each correct CODEC read contains a 50adapter and a possible 30 adapter (in sequencing orientation). The R1's SID is used as the template to trim the R1's 50 adapter and the reverse complement of R2's SID is used to trim R1's 30 adapter, and vice versa for trimming R2. Again, SW algorithm is used to find a match. The reads are grouped based on if the 50 adapter is found on both R1 and R2. In other words, only read pairs with 50 adapters found in both are considered as potential correct reads. However, a few byproducts can also satisfy this criterion. Therefore, it is important to check the 30 adapter if it exists. If a 30 adapter is found and the insert part is too small (e.g., <15 bp), the read is discarded. If both R1 and R2 are discarded, this template is considered as a blank ligation. If only one of the read ends is discarded, it is classified as a double ligation. The summary of byproducts formation and quantification is made by a custom python script also available at the CODECsuite github site.

### ReadPair/Duplex Consensus.

CODECsuite can generate de novo or reference-based consensus. The reference-based consensus has better accuracy and is used throughout this Example. A consensus base is formed if two aligned bases (or gaps in terms of insertion or deletion) agree and N otherwise. CODECsuite keeps the pair-end reads but replaces the read sequence with consensus sequence for both R1 and R2. The sequence quality and other auxiliary tags such as UMI are kept intact. The consensus is generated at uBAM format.

### Alignment Accuracy.

CODECsuite provides a handy and fast tool for evaluating base level accuracy after alignment. It evaluates bases within bed file regions (such as GIAB high confidence regions) and masks against variants in the VCF and/or MAF file, usually for germline variants and somatic variants respectively. It filters at read level (e.g., mapq or edit distances) and base level (by base quality). It also provides abilities to trim from both fragment ends, and evaluates only the overlapping part of the paired reads. It computes accuracy on fragments, cycle and sample levels. For all non-reference bases, it can output details such as base substitutions, quality score, positions on read and reference so that a post processing script can generate error rate by monomer context.

### Duplex Sequencing Data Processing.

Duplex Sequencing data processing used in this Example has been described previously [16,31]. Briefly, Fgbio was used to generate duplex consensus and to filter the consensus reads. The entire workflow and more details are available at the CODECsuite github. Read families with at least 2 copies of each strand were required for generating duplex consensus except for Duplex Sequencing WGS, which relaxed the requirement to 1 copy of each strand to get the best possible duplex recovery.

### Duplex Recovery and Downsample to Certain Family Sizes.

Two custom python scripts were used to generate FIG. 8C and FIG. 6B, respectively. For duplex recovery, the pre-consensus family-assigned reads (after Fgbio GroupReadsByUmi) per target were subsampled at log spaced fractions starting from 10⁻⁴(np.logspace(−4, 0, 30)) and calculated the number of duplex formed at each downsample fraction. This allowed for comprehension of situations where only limited sequencing was given (e.g., <100 read pairs). To understand the impact of family size on error rate, another python script for downsampling was written. In this sample, the number of duplex consensus having the exact family sizes (number of pre-collapsed raw reads) were limited and thus gave less confident results. Thus, families with strictly larger family sizes were used and downsampled to the target family size. It was also sought to maintain an equal or close ratio between the number of reads from each strand.

### Error Rates in Capture Sequencing.

Throughout this, the error rate was defined as substitution error rate at the base level after mapping to the reference genome (hg19). The substitution error rate for calculating the general error rates was used because Illumina sequencers usually generate 100-fold less indel errors and this definition is compliant with what other studies have reported [15]. For panel sequencing with match normal, Miredas were used to calculate the error rate in concordance with previous work [16]. The duplex BAMs from both cfDNA and matched normal samples were generated in the same way and were applied to the same set of filters: 1. no secondary and supplementary alignments; 2. Mapq≥60; 3. Levenshtein distance (L-distance) between the reads excluding soft clipping and reference genome≤5 and number of non N-base L-distance ≤2; 4. Excluding bases within 12 bp distance from both fragment ends. In order not to confuse errors with real mutations, the germline SNVs were pre-computed and GATK4 (HaplotypeCaller) was used from the Duplex Sequencing normal samples as they have higher on-target ratio and hence coverage (89% vs 40% of CODEC). For the patient sample, three somatic SNVs (median VAF=0.26, range 0.24-0.28) were found in the captured regions (Table 3) using MuTect [32].

TABLE 3
Somatic SNVs of patient 315 found in IDT pan-cancer panel (800 kb).

| | | | Reference_ | Tumor_ | tumor_ | tumor_ | tumor_ | normal_ | normal_ |
|---|---|---|---|---|---|---|---|---|---|
| Hugo_Symbol | Chromosome | Start_Position | Allele | Seq_Allele2 | fraction | ref_count | alt_count | ref_count | alt_count |
| PIK3CA | 3 | 178936091 | G | C | 0.245614 | 43 | 14 | 178 | 0 |
| CDKN2A | 9 | 21971000 | C | A | 0.264151 | 39 | 14 | 158 | 1 |
| ARHGAP35 | 19 | 47507737 | C | T | 0.28125 | 46 | 18 | 140 | 0 |

Those somatic mutations (patient sample only) and germline mutations were masked when calculating the error rates. The error rates were only reported for cfDNA samples and the match normal were used for filtering possible germline (failed to call or did not pass quality filter by HaplotypeCaller) and CHIP. Thereby any SNV positions were also masked where there were at least 1 duplex read support in match normal samples as CHIP can occur at very low mutation frequency. Finally, the specificity checks [16] were performed on cfDNA samples to remove substitutions that may rise from alignment errors.

### Error Rate in Whole Genome Sequencing.

The WGS error rate was computed similarly to capture data, except for a few differences. 1, ‘codec accuracy’ was used, a C++ program, as a replacement for Miredas due to its speed improvement. 2, v3.3.2 GIAB NA12878 high confidence VCF and BED file were used as germline masks and evaluation regions. 3, there was no match normal. 4, specificity checks were forgone as it was also very slow for large genomes. Germline SNV and small indel calling in downsampled WGS. The HiSeq 2500 Rapid Run and NovaSeq SP CODEC data were merged to evaluate germline variant calling. The merged CODEC and standard WGS NA12878 samples were downsampled to 1 to 10× (step size 1×) median coverage in the high confidence regions using GATK DownsampleSam. Next, GATK4.1.4.1 best practices pipeline was run via Cromwell and Terra workflow (available at web resources) and computed on the Google Cloud Platform. RTG vcfeval was used to calculate False Positives (FP) and False Negatives (FN) for SNVs and indels (<50 bp) without penalizing genotyping error (if heterozygous variants are called as homozygous and vice versa) using v3.3.2 high confidence VCF and BED file as input. FP per million bases was then calculated by normalizing against the high confidence region size and FN ratio by dividing FN by the total number of true variants.

### Microsatellite Instability Detection.

The full-coverage CODEC consensus BAM and full-coverage standard NGS R1R2 consensus BAM on NA12878 were compared against each other to demonstrate CODEC ability to correct PCR stutter errors and thus to reduce background noise for MSI detection. MSIsensor-pro was used to scan the hg19 for homopolymers of size 8-18 nt. Since MSIsensor-pro does not have mapping quality or secondary alignments filters, the BAM was pre-filtered using SAMtools by requiring mapq≥60 and no secondary or supplementary alignments. And then it was used again to count the number of reads that support different lengths of homopolymer at those pre-selected sites. Any homopolymer sites that overlap or are in close proximity (+1-5 bp) with any germline variants were removed. After that, the reference lengths of the homopolymer sites were considered as true lengths. And observed length distributions from reads were compared against truth. The results were generated from chromosome 1 only.

### References Related to Example 1

[1] Lennon, A. M. et al. Feasibility of blood testing combined with PET-CT to screen for cancer and guide intervention. Science 369, eabb9601 (2020).
[2] Vasan, N., Baselga, J. & Hyman, D. M. A view on drug resistance in cancer. Nature 575, 299-309 (2019).
[3] Griffith, O. L. et al. The prognostic effects of somatic mutations in ER-positive breast cancer. Nat. Commun. 9, 3476 (2018).
[4] Gerlinger, M. et al. Intratumor Heterogeneity and Branched Evolution Revealed by
Multiregion Sequencing. N. Engl. J. Med. 366, 883-892 (2012).
[5] D′Gama, A. M. & Walsh, C. A. Somatic mosaicism and neurodevelopmental disease.
Nature Neuroscience 21, 1504-1514 (2018).
[6] Blauwkamp, T. A. et al. Analytical and clinical validation of a microbial cell-free DNA sequencing test for infectious disease. Nat. Microbiol. 4, 663-674 (2019).
[7] Brazhnik, K. et al. Single-cell analysis reveals different age-related so-matic mutation profiles between stem and differentiated cells in human liver. Sci. Adv. 6, (2020).
[8] Wenger, A. M. et al. Accurate circular consensus long-read sequencing improves variant detection and assembly of a human genome. Nat. Biotechnol. 37, 1155-1162 (2019).
[9] Karst, S. M. et al. High-accuracy long-read amplicon sequences using unique molecular identifiers with Nanopore or PacBio sequencing. Nat. Methods 18, 165-169 (2021).
[10] Shendure, J. et al. DNA sequencing at 40: past, present and future. Nature 550, 345-353 (2017).
[11] Arbeithuber, B., Makova, K. D. & Tiemann-Boege, I. Artifactual mutations resulting from DNA lesions limit detection levels in ultrasensitive sequencing applications. DNA Res. 23, 547-559 (2016).
[12] Kinde, I., Wu, J., Papadopoulos, N., Kinzler, K. W. & Vogelstein, B. Detection and quantification of rare mutations with massively parallel sequencing. Proc. Natl. Acad. Sci. U.
S. A. 108, 9530-9535 (2011).
[13] Schmitt, M. W. et al. Detection of ultra-rare mutations by next-generation sequencing.
Proc. Natl. Acad. Sci. U.S.A 109, 14508-14513 (2012).
[14] Hoang, M. L. et al. Genome-wide quantification of rare somatic mutations in normal human tissues using massively parallel sequencing. Proc. Natl. Acad. Sci. U.S.A 113, 9846-9851 (2016).
[15] Abascal, F. et al. Somatic mutation landscapes at single-molecule resolution. Nature 593, 405-410 (2021).
[16] Parsons, H. A. et al. Sensitive Detection of Minimal Residual Disease in Patients Treated for Early-Stage Breast Cancer. Clin. cancer Res. 26, 2556-2564 (2020).
[17] Pel, J. et al. Duplex Proximity Sequencing (Pro-Seq): A method to improve DNA sequencing accuracy without the cost of molecular bar-coding redundancy. PLoS One 13, 1-19 (2018).
[18] Cohen, J. D. et al. Detection of low-frequency DNA variants by targeted sequencing of the Watson and Crick strands. Nat. Biotechnol. (2021). doi:10.1038/s41587-021-00900-z
[19] Gregory, M. T. et al. Targeted single molecule mutation detection with massively parallel sequencing. Nucleic Acids Res. 44, e22 (2016).
[20] Wang, K. et al. Ultrasensitive and high-efficiency screen of de novo low-frequency mutations by o2n-seq. Nat. Commun. 8, 15335 (2017).
[21] Lou, D. I. et al. High-Throughput DNA sequencing errors are reduced by orders of magnitude using Circle Sequencing. Proc. Natl. Acad. Sci. U.S.A 110, 19872-19877 (2013).
[22] Kircher, M., Sawyer, S. & Meyer, M. Double indexing overcomes inaccuracies in multiplex sequencing on the Illumina platform. Nucleic Acids Res. 40, e3-e3 (2012).
[23] Xiong, K. et al. Duplex-Repair enables highly accurate sequencing, despite DNA damage. bioRxiv (2021) doi:10.1101/2021.05.21.445162.
[24] Kim, S. et al. Deamination Effects in Formalin-Fixed, ParaffinEmbedded Tissue Samples in the Era of Precision Medicine. J. Mol. Diagnostics 19, 137-146 (2017).
[25] Zook, J. M. et al. An open resource for accurately benchmarking small variant and reference calls. Nat. Biotechnol. 37, 561-566 (2019).
[26] DePristo, M. A. et al. A framework for variation discovery and genotyping using next-generation DNA sequencing data. Nat. Genet. 43, 491-498 (2011).
[27] Yu, F. et al. NGS-based identification and tracing of microsatellite instability from minute amounts DNA using inter-Alu-PCR. Nucleic Acids Res. 49, e24-e24 (2021).
[28] Maruvka, Y. E. et al. Analysis of somatic microsatellite indels identifies driver events in human tumors. Nat. Biotechnol. 35, 951-959 (2017).
[29] Benjamin, D. et al. Calling Somatic SNVs and Indels with Mutect2. bioRxiv 861054 (2019). doi:10.1101/861054
[30] Tate, J. G. et al. COSMIC: the Catalogue Of Somatic Mutations In Cancer. Nucleic Acids Res. 47, D941-D947 (2019).
[31] Gydush, G. et al. MAESTRO affords ‘breadth and depth’ for mutation testing. bioRxiv (2021) doi:10.1101/2021.01.22.427323.
[32] Cibulskis, K. et al. Sensitive detection of somatic point mutations in impure and heterogeneous cancer samples. Nat. Biotechnol. 31, 213-219 (2013).
[33] May, A., Abeln, S., Crielaard, W., Heringa, J. & Brandt, B. W. Un-raveling the outcome of 16S rDNA-based taxonomy analysis through mock data and simulations. Bioinformatics 30, 1530-1538 (2014).
[34] Limasset, A., Flot, J. F. & Peterlongo, P. Toward perfect reads: Self-correction of short reads via mapping on de Bruijn graphs. Bioinfor-matics 36, 1374-1381 (2020).
[35] Smith, T. F. & Waterman, M. S. Identification of common molecular subsequences. J. Mol. Biol. 147, 195-197 (1981).

A CODEC (CDS) adapter complex has been designed, which consists of four oligonucleotides (oligos) hybridized, to include every element required for both concatenation and adapter attachment. In certain embodiment, in order to stay as a whole, it is critical that lengths and hybridization ΔG° of double-stranded regions (1 and 4) are strong enough. Based on DNA hybridization thermodynamics, region 1 was designed to have >15 bp and <−20 kcal/mol which worked well. Region 4 was given extra length (30 bp) as it needs to hold two oligos.

### Example 2—Methylation-Specific CODEC Sequencing

This Example describes an embodiment referred to as “methylation-specific CDS” (or equivalently, “methylation-specific CODEC”) which can be used for performing improved mutation and methylation sequencing of DNA samples.

This embodiment enables extraction of information about DNA methylation, as well as mutation, from the interrogated DNA sample. There has been increasing interest in extracting DNA methylation information from clinical samples in several fields, including cancer. For example, extracting cancer-specific fingerprints of methylated DNA from liquid biopsies have recently led to approaches for early detection of multiple cancers

To enable extraction of methylation information from a DNA sample and to perform methylation-sensitive sequencing, in most cases a chemical or enzymatic de-amination step is applied to the sample prior to performing sample amplification. This step enables selective conversion of un-methylated cytosines to uracils, while methylated cytosines remain unchanged. Following this step, amplification of the sample with standard deoxynucleotides (dNTPs) results to conversion of unmethylated cytosines to thymidines, while methylated cytosines become cytosines. Subsequent sequencing enables to infer which cytosines in the original sample were methylated or un-methylated.

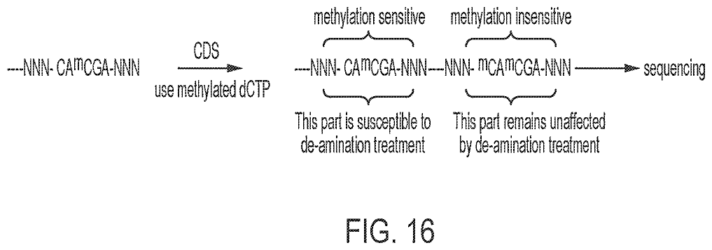

To enable CODEC to retain and report DNA methylation information, the following protocol has been developed, as represented in FIG. 16.

The protocol involves the following steps:
(a) Synthesize the CODEC adaptor-complex to contain methylated cytosine, instead of regular, unmethylated cytosine. In this way they are refractory to subsequent de-amination and can be amplified with the described primers.
(b) Following ligation of the modified CODEC adaptors, a copy of the opposite DNA strand is generated using methylated-dCTP along with standard dATP, dGTP and dTTP nucleotides. In this way the copy of the original strand is always methylated at cytosine positions and becomes refractory to subsequent de-amination.
(c) Conduct a deamination step to convert un-methylated cytosines to uracils in the original top DNA strand. The deamination of cytosines can be performed with one of several approaches, such as the standard bisulfite-de-amination²; enzymatic deamination using enzymatic methyl-seq (EM-seq) technique, which uses enzymatic steps by TET2 and APOBEC2 enzymes to differentiate between methylated and un-methylated cytosine³. Or the recently reported TET Assisted Pic-borane Sequencing, TAPS method⁴.
(d) Following the deamination step, amplification using the CODEC adaptor primers is applied.
(e) Conduct duplex sequencing.

By generating a copy of the original DNA strand, which is insensitive to de-amination, while retaining the methylation/unmethylation information in the original strand, it is now possible to infer methylation sequencing information as well as mutation information by comparison of the sequencing results obtained from the two strands. For example, if a C is present in the copied strand and a C is also present in the original strand, one can infer that this sequence position was methylated in the original sample. While if there is a T in the original strand then this sequence position was probably un-methylated in the original sample. (To exclude the possibility that the T appears because of sequencing error, additional analysis may need to be done: for example, one can observe the nucleotide context in which this T appears on the original strand. If additional Ts also appear nearby, then the T likely represents an unmethylated C; if it is an isolated T then there is a good probability the T is a result of sequencing error).

Creating a methylation-insensitive second DNA strand copy in the CODEC protocol along with the original methylation-sensitive DNA strand has several possible practical applications.

For example, since the copied DNA strand by preserving the cytosines at all positions is not ‘cytosine poor’ it can be used for unambiguous alignment during sequencing, thus enabling enhanced mapping of sequence reads. Also, the methylation insensitive strand can be used for improved hybrid capture since DNA strands with multiple un-methylated sites are often problematic for hybrid capture. Also, it can be used to improve proof-reading of sequence calls and for general duplex sequencing correction on other bases. Finally, it can be used to create libraries that preserve both mutation and methylation information for subsequent combined ‘methyl-mutation’ sequencing using a single DNA sample (instead of using two separate samples, one for mutation and another for methylation analysis).

Synthesizing the opposite strand using methylated dCTP and followed by de-amination of un-methylated cytosines has some advantages such as: 1) unambiguous alignment, since all 4 bases are present, this preserves sequence diversity and enhances the ability to align sequences, 2) improved hybrid capture, even for un-methylated sites which are often a problem, 3) improved proof-reading of sequence calls on the methylation-sensitive portion and for general duplex sequencing correction on other bases, and 4) creating a library for subsequent combined methyl-mutation sequencing using a single DNA sample (instead of two separate samples).

### References for Example 2

[1] Cohen J D, Li L, Wang Y, Thoburn C, Afsari B, Danilova L, Douville C, Javed A A, Wong F, Mattox A, Hruban R H, Wolfgang C L, Goggins M G, Dal Molin M, Wang T L, Roden R, Klein A P, Ptak J, Dobbyn L, Schaefer J, Silliman N, Popoli M, Vogelstein J T, Browne J D, Schoen R E, Brand R E, Tie J, Gibbs P, Wong H L, Mansfield A S, Jen J, Hanash S M, Falconi M, Allen P J, Zhou S, Bettegowda C, Diaz L A, Jr., Tomasetti C, Kinzler K W, Vogelstein B, Lennon A M, Papadopoulos N: Detection and localization of surgically resectable cancers with a multi-analyte blood test. Science 2018, 359:926-30.
[2] Frommer M, McDonald L E, Millar D S, Collis C M, Watt F, Grigg G W, Molloy P L, Paul C L: A genomic sequencing protocol that yields a positive display of 5-methylcytosine residues in individual DNA strands. Proc Natl Acad Sci USA 1992, 89:1827-31.
[3] Vaisvila R, Ponnaluri VKC, Sun Z, Langhorst B W, Saleh L, Guan S, Dai N, Campbell M A, Sexton B, Marks K, Samaranayake M, Samuelson J C, Church H E, Tamanaha E, Correa I R, Pradhan S, Dimalanta E T, Evans T C, Williams L, Davis T B: E M-seq: Detection of DNA Methylation at Single Base Resolution from Picograms of DNA. bioRxiv 2019:2019.12.20.884692.
[4] Liu Y, Siejka-Zielinska P, Velikova G, Bi Y, Yuan F, Tomkova M, Bai C, Chen L, Schuster-Böckler B, Song C X: Bisulfite-free direct detection of 5-methylcytosine and 5-hydroxymethylcytosine at base resolution. Nat Biotechnol 2019, 37:424-9.

### Example 3—Combination of Duplex-Repair and CODEC Sequencing

As illustrated in FIG. 1AF of the present disclosure, it is contemplated that CODEC sequencing may be combined with Duplex-Repair, which aims to minimize the presence of false mutations prior to CODEC sequencing. Duplex-Repair may be used in place of end repair/dA tailing (ER/AT) methods known in the art. This Example describes Duplex-Repair.

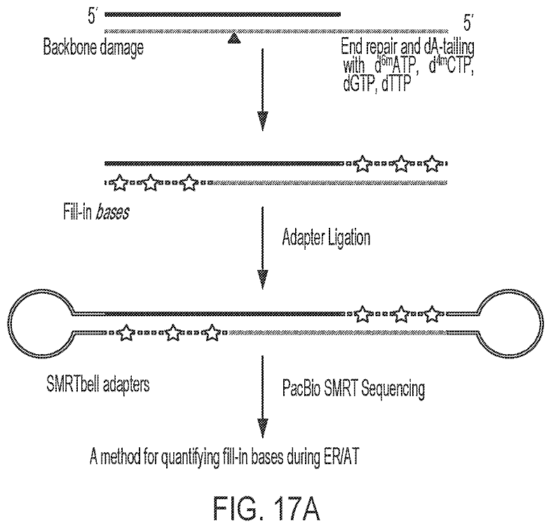

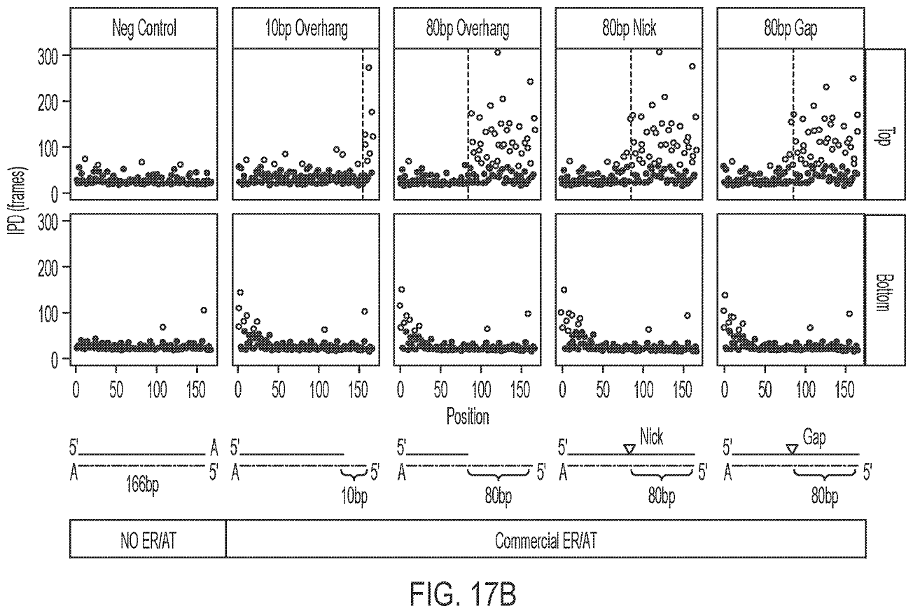

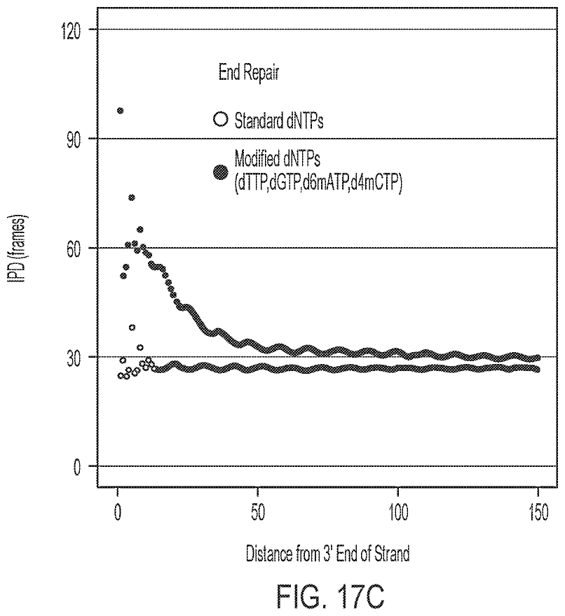

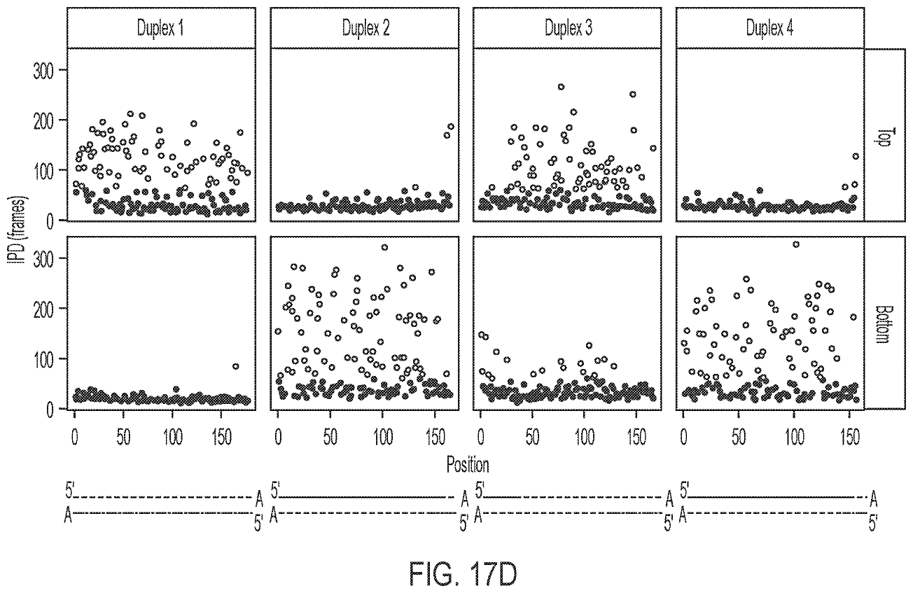

The present disclosure also relates to a new approach for ‘end repair/dA-tailing’ (ER/AT) to minimize strand resynthesis (and thus, the potential to copy base damage errors to both stands prior to NGS adapter ligation). The premise for this technology came from the observation that substantial amounts of strand resynthesis could occur using commercially available ER/AT methods (FIGS. 17A-17D). An assay was first developed to measure strand resynthesis using single molecule real-time sequencing to detect the incorporation of d6mATP and d4mCTP (substituted for standard dATP and dCTP) based on extended interpulse durations (IPDs, FIG. 17A). It was used to verify the hypothesis that extensive strand resynthesis occurs when commercial ER/AT is applied to synthetic oligos bearing nicks, gaps, and overhangs. It is shown that when there are nicks or gaps deep within a strand, all downstream bases are resynthesized (FIG. 17B). (It is also found that T4 polymerase can ‘step back’ a few bases before proceeding forward to resynthesize the strand, and that it can partly resynthesize the bottom strand of each oligo at its blunt end, likely due to DNA breathing. This suggests that resynthesis also occurs, in part, irrespective of backbone damage and may further depend on specific reaction conditions.) This assay was then applied to healthy donor cfDNA and found that while most resynthesis occurs at fragment ends (FIG. 17C), many duplexes were almost entirely resynthesized (FIG. 17D). This is clearly a major problem for duplex sequencing; yet, the single-molecule sequencing assay provides for the resolution needed to characterize and resolve this issue.

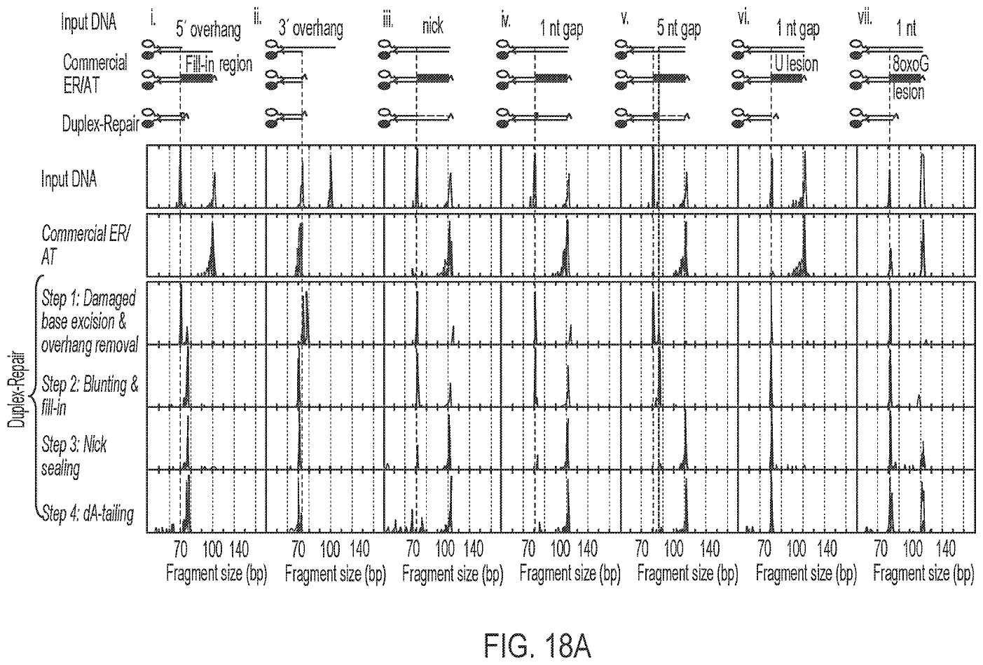

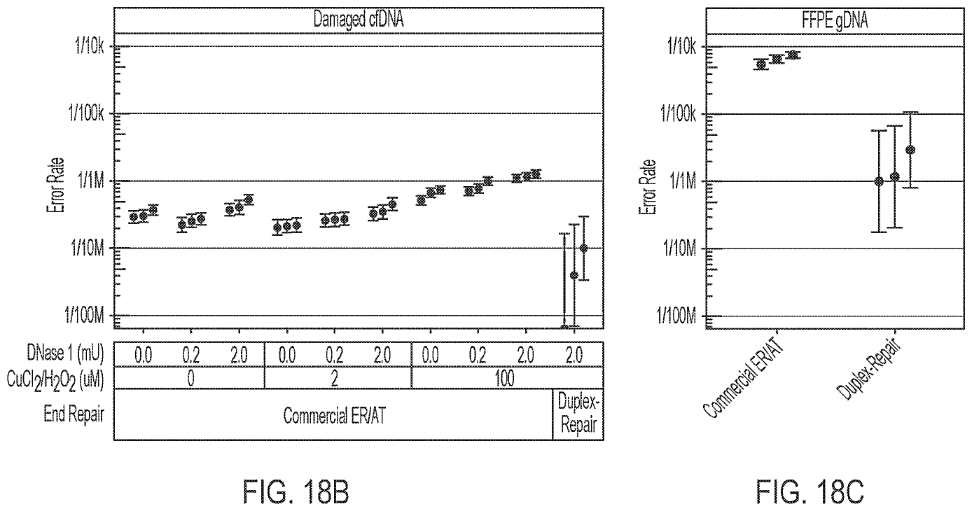

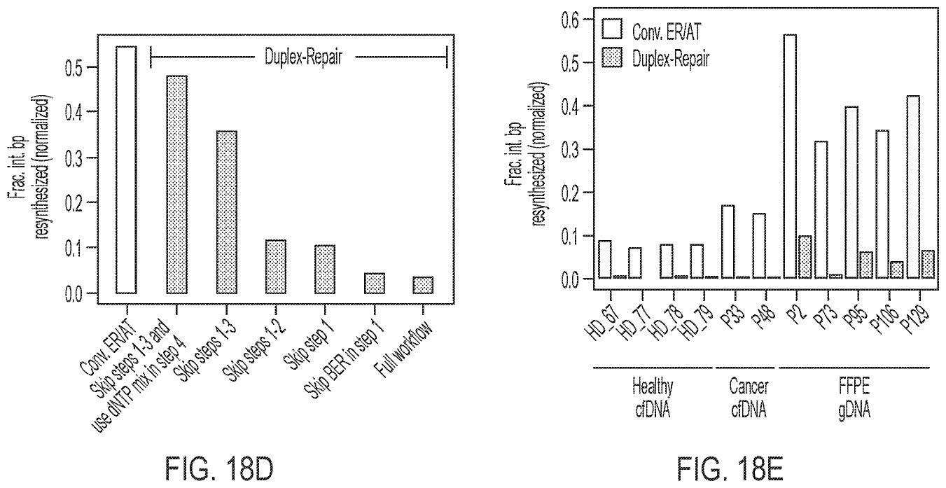

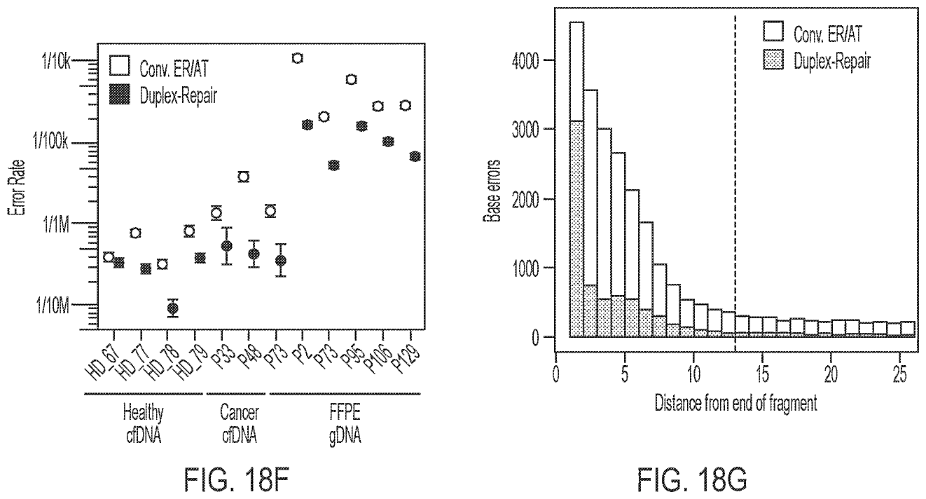

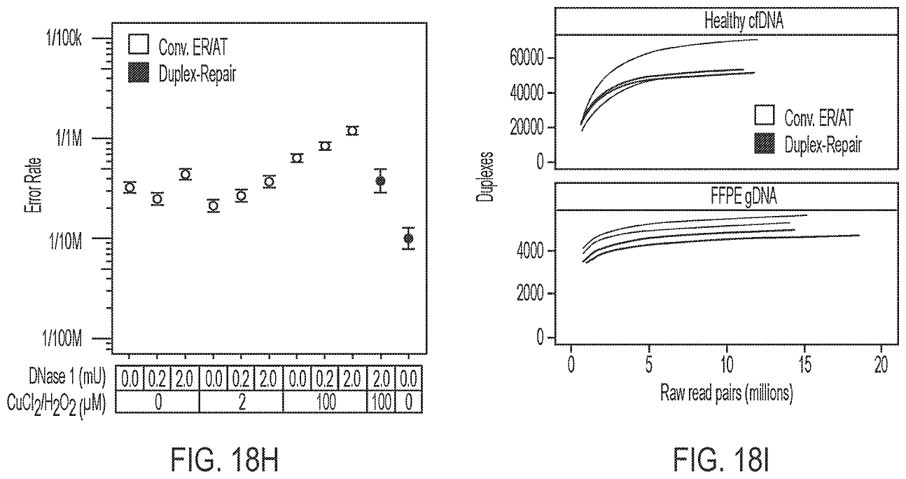

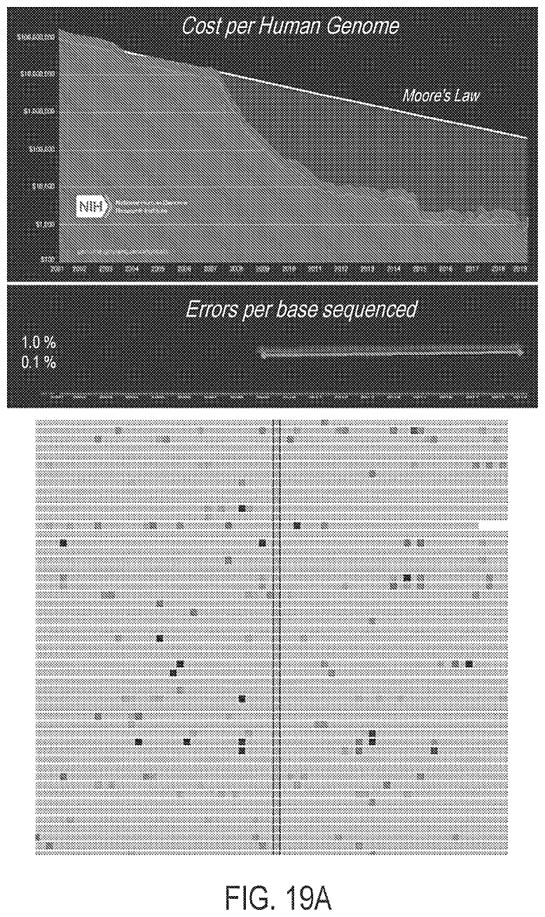

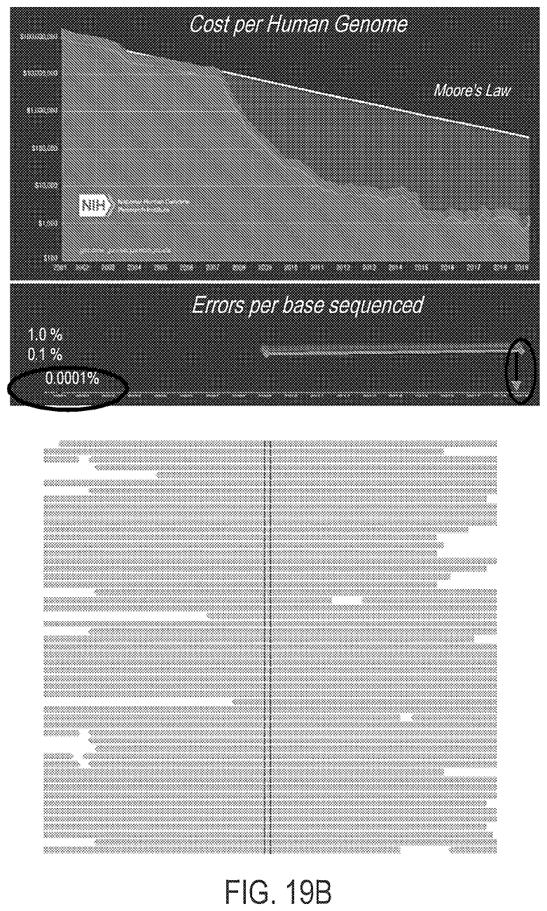

This new method called Duplex-Repair performs ER/AT in a careful and stepwise manner to limit strand resynthesis prior to adapter ligation. Duplex-Repair consists of four major steps: (1) damaged base excision and overhang removal, (2) blunting and restricted fill-in, (3) nick sealing, and (4) dA-tailing (FIG. 18A). DNA is first treated with an enzyme cocktail comprised of Endonuclease IV, Formamidopyrimidine [fapy]-DNA glycosylase, Uracil-DNA glycosylase and T4 pyrimidine DNA glycosylase and Endonuclease VIII, which recognizes and excises damaged bases such as Uracil, 8-oxoG, oxidized pyrimidines, cyclobutane pyrimidine dimers and abasic sites, producing either 1 nt gaps (if within double-stranded segments) or strand breaks (if within single-stranded regions). Exonuclease VII (ExoVII) is also present and degrades 3′ and 5′ single-strand overhangs. In the second step, T4 polynucleotide kinase (de)phosphorylates DNA termini and T4 DNA polymerase (which has 3′ exonuclease but no 5′ exonuclease or strand displacement activity) blunts 3′ overhangs and fills in gaps and short (<7nt) remaining 5′ single-strand overhangs left behind by ExoVII. Then, nicks are sealed by HiFi Taq DNA ligase. Finally, dA-tailing is performed using Klenow fragment (exo-) and Taq DNA polymerase, but with only dATP present, to prevent strand resynthesis. The performance of Duplex-Repair has been verified using multiple synthetic oligonucleotides, reflecting common types of expected backbone damages in real DNA samples (FIG. 18A). For each duplex, the top and bottom strands were labelled with distinct dyes at their 5′ and 3′ ends, respectively, and capillary electrophoresis was used to measure bases added or removed from each under varied treatment conditions. Duplex oligonucleotides bearing (i) 5′ overhangs, (ii) 3′ overhangs, (iii) nicks, (iv-v) gaps of varied lengths without base damage, and (vi-vii) gaps with base damage were evaluated. For all cases except 3′ overhangs, substantial strand resynthesis was shown to occur with commercial ER/AT (Kapa HyperPrep kit), whereas Duplex-Repair significantly reduced the number of bases that were resynthesized. It has been further confirmed that Duplex-Repair limits the impact of varied base and backbone damages on duplex sequencing accuracy. A large amount of cfDNA was first collected from one healthy donor and treated multiple aliquots of it with varied amounts of CuCl2/H2O2 (to induce oxidative damage) and DNase I (to create nicks). A commercial ER/AT kit (the Kapa HyperPrep kit) was then applied and showed that errors increased by an order of magnitude in the most heavily damaged condition (FIG. 18B). (Intriguingly, the increase in error rate with DNase I concentration suggests that for liquid biopsy tests using commercial ER/AT kits, the reliability of genomic analyses may depend in part upon the levels of DNase I, among other extracellular nucleases, in a patient's bloodstream.) Duplex-Repair was then applied to the most heavily damaged condition and found that it ‘rescued’ the impact of base and backbone damage and provided even lower error rates than the undamaged cfDNA samples which were prepared using commercial ER/AT (FIG. 18B). Similar results were observed for formalin fixed tumor biopsies (FIG. 18C). Considering that base and backbone damage can arise spontaneously (e.g. cytosine deamination) and in response to environmental and chemical exposures (e.g. UV radiation, reactive oxygen species, formalin fixation, freeze-thaw, heating, acoustic shearing, etc.), Duplex-Repair is needed to ensure the reliability of duplex sequencing for a wide range of samples.

One aspect of the present disclosure relates to optimizing Duplex-Repair to correct backbone damage in duplex DNA with minimal strand resynthesis and maximum library conversion efficiency (i.e., the fraction of DNA duplexes converted into adapter-ligated library molecules). It is shown that Duplex-Repair minimizes strand resynthesis and protects against translesion synthesis in ER/AT, but the current protocol involves multiple buffer exchanges which yield fewer total duplexes and explains the wider error bars on Duplex-Repair samples in FIGS. 18B-18C. Here, Duplex-Repair is formulated into the fewest possible steps (e.g., eliminating ‘clean-ups’ in between steps) and optimize buffer compositions and experimental conditions (e.g., time, temperature, concentration, and alternative enzymes) such that multiple enzymes can function together. Performance is verified using (i) a single molecule sequencing assay (FIG. 17A), (ii) synthetic oligonucleotide substrates and capillary electrophoresis (FIG. 18A), and real DNA samples sequenced following ER/AT (FIGS. 18B-18C). Both qPCR with library primers and NGS to are used quantify conversion efficiency using varied inputs (e.g., 1-1000ng) of buffy-coat-derived genomic DNA, from a healthy donor whose germline sequence has been determined, sheared to different median insert sizes (e.g., 50-250 bp) with different methods (e.g. sonication, enzymatic digestion). The workflow has been tested on challenging samples such as DNA subjected to varied extents of base and backbone damage (FIG. 18B) and formalin fixed tumor biopsies (FIG. 18C).

Duplex-Repair provides consistently high accuracy in duplex sequencing irrespective of the extent of base and backbone damage in sample. This helps to ensure that NGS results are robust for all clinical samples. Duplex-Repair still requires some amount of DNA polymerization to fill gaps and short overhangs left behind after ExoVII treatment, for instance. This means there is still a need to trim fragment ends in silico, up to about 8-12 bases, which will reduce data output, but is necessary to safeguard against false discovery. Each polymerase has a different propensity for translesion synthesis, while there are many types of base damages that could arise. For base damage to generate an error in duplex sequencing, it must be able to be copied by polymerases in both ER/AT and library amplification. The propensity of each polymerase to bypass common base damages (e.g., 8-oxoguanine, uracil, abasic sites, etc.) and insert the ‘wrong’ base will be tested. However, there are a large number of possible base damages that can arise in DNA, and it will be impossible to test all such lesions. Further, each enzyme will not be 100% efficient; it is therefore expected to incur some loss of DNA product. The enzymes and reaction conditions that provide highest efficiency in each step will be identified, using synthetic oligos and capillary electrophoresis (FIG. 18A). Future strategies for DNA fragmentation could limit the need for ER/AT if, for instance, overhang length could be limited, or adapters directly added via tagmentation or ligation at double-stranded breaks. These methods will be explored. However, it is still expected that there will be a need for ER/AT to correct backbone damages that arise naturally in DNA, and maximize duplex recovery. For instance, there are many clinical specimens such as cfDNA which are already fragmented, for which ER/AT will always be needed.

### Equivalents and Scope

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following embodiments.

What is claimed is:

1. An isolated nucleic acid complex (complex) comprising at least ten (10) regions (R01-R10) in the following configuration: 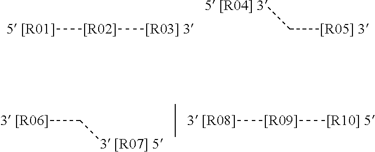
wherein, [symbol] represents bonding,
wherein R01, R02, and R03 comprise a first oligonucleotide,
wherein R04 and R05 comprise a second oligonucleotide,
wherein R06 and R07 comprise a third oligonucleotide,
wherein R08, R09, R10 comprise a fourth oligonucleotide,
wherein, R01 and R06 are annealed to one another,
wherein, R03 and R08 are annealed to one another,
wherein, R05 and R10 are annealed to one another,
wherein, R02 and R07 are not annealed to one another, and
wherein, R04 and R09 are not annealed to one another;
wherein R02 comprises a single-stranded linker, first unique molecular identifier (UMI), and a first read primer site, and
wherein R09 comprises a single-stranded linker, a second UMI, and a second read primer site.

2. The complex of claim 1, wherein:
(1) R01 comprises a first adapter;
(2) R02 comprises a single-stranded linker, first unique molecular identifier (UMI), and a first read primer site;
(3) R03 comprises a first sequence at or near the 3′ end capable of priming DNA synthesis by a DNA-dependent DNA polymerase;
(4) R04 comprises a free 5′ end comprising a first next-generation sequencing (NGS) adapter sequence;
(5) R05 comprises a third adapter and a first sample index;
(6) R06 comprises a second adapter and a second sample index;
(7) R07 comprises a free 5′ end comprising a second next-generation sequencing (NGS) adapter sequence;
(8) R08 comprises a second sequence at or near the 3′ end capable of priming DNA synthesis by a DNA-dependent DNA polymerase;
(9) R09 comprises a single-stranded linker, a second UMI, and a second read primer site; and/or
(10) R10 comprises a fourth adapter.

3. The complex of claim 2, wherein the first sequence and second sequence, further comprise the same or different primer binding sites.

4. The complex of claim 2 or any one of claims 2-3, wherein the first and second primer sites are oriented to initiate sequencing by addition in opposing directions.

5. The complex of any one of claims 1-4, wherein the first and second UMI are distinct.

6. The complex of any one of claims 1-5, wherein:
R01 comprises at least 12 nucleotides,
R02 comprises at least 14 nucleotides,
R03 comprises at least 12 nucleotides,
R04 comprises at least 20 nucleotides,
R05 comprises at least 12 nucleotides,
R06 comprises at least 12 nucleotides,
R07 comprises at least 20 nucleotides,
R08 comprises at least 12 nucleotides,
R09 comprises at least 14 nucleotides, and/or
R10 comprises at least 12 nucleotides.

7. The complex of any one of claims 1-6, wherein:
R01 comprises less than 30 nucleotides,
R02 comprises less than 75 nucleotides,
R03 comprises less than 99 nucleotides,
R04 comprises less than 49 nucleotides,
R05 comprises less than 30 nucleotides,
R06 comprises less than 30 nucleotides,
R07 comprises less than 49 nucleotides,
R08 comprises less than 99 nucleotides,
R09 comprises less than 75 nucleotides, and/or
R10 comprises less than 30 nucleotides.

8. The complex of any one of claims 1-7, wherein:
R01 comprises between 12 and 30 nucleotides,
R02 comprises between 14 and 75 nucleotides,
R03 comprises between 12 and 99 nucleotides,
R04 comprises between 20 and 49 nucleotides,
R05 comprises between 12 and 30 nucleotides,
R06 comprises between 12 and 30 nucleotides,
R07 comprises between 20 and 49 nucleotides,
R08 comprises between 12 and 99 nucleotides,
R09 comprises between 14 and 75 nucleotides, and/or
R10 comprises between 12 and 30 nucleotides.

9. The complex of any one of claims 1-8, wherein:
(a) R01 and R06 comprise a hybridization free energy of about −10 kcal/mol, about −15 kcal/mol, about −20 kcal/mol, about −25 kcal/mol, about −30 kcal/mol, or about −35 kcal/mol;
(b) R03 and R08 comprise a hybridization free energy of about −10 kcal/mol, about −15 kcal/mol, about −20 kcal/mol, about −25 kcal/mol, about −30 kcal/mol, about −35 kcal/mol, about −40 kcal/mol, about −45 kcal/mol, about −50 kcal/mol, about −55 kcal/mol, about −60; and/or
(c) R05 and R10 comprise a hybridization free energy of about −10 kcal/mol, about −15 kcal/mol, about −20 kcal/mol, about −25 kcal/mol, about −30 kcal/mol, or about −35 kcal/mol.

10. The complex of any one of claims 1-9, wherein:
(a) R01 and R06 each comprise the same number of nucleotides, optionally wherein R06 has a one nucleotide overhang to facilitate ligation;
(b) R03 and R08 each comprise the same number of nucleotides; and/or
(c) R05 and R10 each comprise the same number of nucleotides, optionally wherein R05 has a one nucleotide overhang to facilitate ligation.

11. The complex of any one of claims 1-10, wherein:
(a) R01 and R06 comprise sequences with at least 90% complementarity;
(b) R03 and R08 comprise sequences with at least 90% complementarity; and/or
(c) R05 and R10 comprise sequences with at least 90% complementarity.

12. The complex of any one of claims 1-11, wherein each R01, R06, R05, and R10 comprise the same number of nucleotides, optionally wherein R06 and R05 each have a one nucleotide overhang to facilitate ligation.

13. The complex of any one of claims 2-12, wherein the complex comprises at least two elements according to claim 2.

14. The complex of any one of claims 2-13, wherein the complex comprises at least three elements according to claim 2.

15. The complex of any one of claims 2-14, wherein the complex comprises at least four elements according to claim 2.

16. The complex of any one of claims 2-15, wherein the complex comprises at least five elements according to claim 2.

17. The complex of any one of claims 2-16, wherein the complex comprises at least six elements according to claim 2.

18. The complex of any one of claims 2-17, wherein the complex comprises at least seven elements according to claim 2.

19. The complex of any one of claims 2-18, wherein the complex comprises at least eight elements according to claim 2.

20. The complex of any one of claims 2-19, wherein the complex comprises at least nine elements according to claim 2.

21. The complex of any one of claims 1-20, wherein:
(1) R01 comprises a first concatenated duplex sequencing (CDS) adapter;
(2) R02 comprises a single-stranded linker;
(3) R03 comprises a 3′ end capable of priming DNA synthesis by a DNA-dependent DNA polymerase;
(4) R04 comprises a first unique molecular identifier (UMI);
(5) R05 comprises a third CDS adapter;
(6) R06 comprises a second CDS adapter;
(7) R07 comprises a second UMI;
(8) R08 comprises a 3′ end capable of priming DNA synthesis by a DNA-dependent DNA polymerase;
(9) R09 comprises a single-stranded linker; and
(10) R10 comprises a fourth CDS adapter.

22. The complex of any one of claims 1-21,
wherein the 5′ end of R01 is ligated to the 3′ end of a first strand of a target DNA duplex;
wherein the 3′ end of R05 is ligated to the 5′ end of the first strand of the target DNA duplex;
wherein the 5′ end of R10 is ligated to the 3′ end of a second strand of the target DNA duplex;
wherein the 3′ end of R06 is ligated to the 5′ end of the second strand of the target DNA duplex;
forming a circularized DNA duplex or optionally a partially double-stranded circular DNA.

23. The isolated nucleic acid complex according to any of claims 1-22 for use in next-generation sequence of a DNA sample.

24. The isolated nucleic acid complex according to any of claims 1-22 for use in place of a duplex adapter in a next generation sequencing workflow to obtain the sequence of a DNA sample.

25. A sequencing adapter having a first end, a second end and a central portion positioned between the first and second ends, wherein the first end comprises a first duplex comprising a first oligonucleotide annealed to a second oligonucleotide, wherein the second end comprises a second duplex comprising a third oligonucleotide annealed to a fourth oligonucleotide, and wherein the second and the fourth oligonucleotides are annealed to one another over a region complementarity to form a third duplex that is positioned in the central portion, wherein the sequencing adapter further comprises a pair of read primer binding sites on either side of the third duplex in single stranded regions.

26. The sequencing adapter of claim 25, wherein the first duplex is 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, or 40 bp in length.

27. The sequencing adapter of claim 25, wherein the first duplex has hybridization free energy of about −10 kcal/mol, about −15 kcal/mol, about −20 kcal/mol, about −25 kcal/mol, about −30 kcal/mol, or about −35 kcal/mol.

28. The sequencing adapter of claim 25, wherein the second duplex is 10 bp, 11 bp, 12 bp, 13 bp, 14 bp, 15 bp, 16 bp, 17 bp, 18 bp, 19 bp, 20 bp, 21 bp, 22 bp, 23 bp, 24 bp, or 25 bp in length.

29. The sequencing adapter of claim 25, wherein the first duplex has hybridization free energy of about −10 kcal/mol, about −15 kcal/mol, about −20 kcal/mol, about −25 kcal/mol, about −30 kcal/mol, or about −35 kcal/mol.

30. The sequencing adapter of claim 25, wherein the third duplex is 10 bp, 11 bp, 12 bp, 13 bp, 14 bp, 15 bp, 16 bp, 17 bp, 18 bp, 19 bp, 20 bp, 21 bp, 22 bp, 23 bp, 24 bp, or 25 bp in length.

31. The sequencing adapter of claim 25, wherein the third duplex has hybridization free energy of about −10 kcal/mol, about −15 kcal/mol, about −20 kcal/mol, about −25 kcal/mol, about −30 kcal/mol, or about −35 kcal/mol.

32. The sequencing adapter of claim 25, wherein the single stranded regions are 5, 6, 7, 8, 9, 10, 11, 12, 1, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

33. The sequencing adapter of claim 25, wherein the first oligonucleotide comprises a free 5′ end comprising a first next-generation sequencing (NGS) flow cell binding region.

34. The sequencing adapter of claim 25, wherein the third oligonucleotide comprises a free 5′ end comprising a second next-generation sequencing (NGS) flow cell binding region.

35. The sequencing adapter of claim 25, wherein the first duplex has a first free 5′ end and the second duplex has a second free 5′ end.

36. The sequencing adapter of claim 25, wherein the third duplex comprises a free 5′ end on each strand of the duplex, wherein the first and second 3′ ends can prime DNA synthesis by a DNA-dependent DNA polymerase.

37. The sequencing adapter according to any of claims 23-36 for use in next-generation sequence of a DNA sample.

38. The sequencing adapter according to any of claims 23-36 for use in place of a duplex adapter in a next generation sequencing workflow to obtain the sequence of a DNA sample.

39. A method of preparing a sequencing library, comprising:
(a) ligating the complex of any one of claims 1-22 to a dsDNA duplex as follows: ligating the 5′ end of R01 to the 3′ end of a first strand of the dsDNA duplex; ligating the 3′ end of R05 to the 5′ end of the first strand of the dsDNA duplex; ligating the 5′ end of R10 to the 3′ end of a second strand of the dsDNA duplex; and ligating the 3′ end of R06 to the 5′ end of the second strand of the dsDNA duplex; thereby forming a circular double-stranded DNA intermediate comprising the target DNA molecule and the complex;
(b) extending a first DNA strand from the 3′ end of R03;
(c) extending a second DNA strand from the 3′ end of R08; and
(d) optionally annealing the first and second DNA strands to form a double-stranded DNA molecule for use in next-generation sequencing (NGS) of the target DNA molecule.

40. The method of claim 39, wherein the double-stranded DNA molecule comprises two copies of the target DNA molecule.

41. The method of claim 39 or claim 40, wherein the ligating of step (a) comprises adding ligase.

42. The method of any one of claims 39-41, wherein the synthesizing of steps (b) and (c) comprise contacting the circular double-stranded DNA intermediate with a polymerase.

43. The method of claim 42, wherein the polymerase is a DNA-dependent DNA polymerase.

44. The method of claim 42 or claim 43, wherein the polymerase has a strand-displacement activity.

45. The method of any one of claims 39-44, wherein the next-generation sequencing (NGS) is a short-read strategy.

46. The method of any one of claims 39-45, further comprising sequencing double-stranded DNA molecule by next-generation sequencing.

47. A method of preparing a sequencing library comprising a plurality of DNA duplexes to be sequenced, comprising for each member of the library:
(a) ligating the first and second ends of a sequencing adapter of any one of claims 25-36 to a sample DNA fragment having opposing top and bottom strands, thereby forming a partially circularized DNA molecule comprising the DNA fragment and the sequencing adapter; and
(b) synthesizing first and second single-strand DNA molecules by extending the free 3′ ends on the sequencing adapter each using the opposite strand of the partially circularized DNA molecule as a template, thereby forming a linearized double-stranded DNA molecule configured for next generation sequencing, said linearized double-stranded DNA molecule comprising a first double-stranded region comprising the original top strand paired with a copied bottom strand, and a second double-stranded region comprising a copied top strand paired with the original bottom strand,
wherein a plurality of linearized double-stranded DNA molecule each prepared from a different DNA fragment constitute the next-generation sequencing library.

48. The method of claim 47, wherein the linearized double-stranded DNA molecule configured for next generation sequencing and having first and second ends comprises the following structure:
first end-[a first next generation flow cell adapter]-[a first duplex region comprising the original top strand paired with a copy of original bottom strand]-[a second duplex region comprising the central portion of the next-generation sequencing adapter]-[a third duplex region comprising a copy of original top strand paired with the original bottom strand]-[a second next generation flow cell adapter]-second end.

49. The method of claim 48, wherein the first next generation flow cell adapter is an Illumina P5 or P7 adapter sequence.

50. The method of claim 48, wherein the second next generation flow cell adapter is an Illumina P5 or P7 adapter sequence.

51. The method of claim 48, wherein the second duplex region comprises first and second read primer binding sites, wherein each first and second read primer sites is further associated with a unique molecule identifier (UMI) and a sample index sequence.

52. The method of claim 51, wherein the first and second read primer binding sites are orientated outwardly towards the ends of the linearized double-stranded DNA molecule.

53. The method of claim 52, wherein a first read primer can be used to obtain a sequence read comprising a UMI, sample index, and the original top strand, or portion thereof, of the sample DNA fragment to be sequenced.

54. The method of claim 52, wherein a second read primer can be used to obtain a sequence read comprising a UMI, sample index, and the original bottom strand, or portion thereof, of the sample DNA fragment to be sequenced.

55. The method of claim 47, wherein the method is used in place of a commercial next-generation library construction kit.

56. The method of claim 47, wherein the ligating of step (a) comprises adding ligase.

57. The method of claim 47, wherein the synthesizing of step (b) comprising adding a DNA polymerase, optionally having a strand-displacement activity.

58. The method of claim 51, further comprising the step of obtaining the sequence of the original top and original bottom strands by conducting next generation sequencing with the first and second read primers.

59. A linearized double-stranded DNA molecule configured for next generation sequencing obtained by the method of claim 47, wherein the linearized double-stranded DNA molecule comprises first and second ends and has the following structure:
first end-[a first next generation flow cell adapter]-[a first duplex region comprising the original top strand paired with a copy of original bottom strand]-[a second duplex region comprising the central portion of the next-generation sequencing adapter]-[a third duplex region comprising a copy of original top strand paired with the original bottom strand]-[a second next generation flow cell adapter]-second end.

60. The linearized double-stranded DNA molecule of claim 59, wherein the first next generation flow cell adapter is an Illumina P5 or P7 adapter sequence.

61. The linearized double-stranded DNA molecule of claim 59, wherein the second next generation flow cell adapter is an Illumina P5 or P7 adapter sequence.

62. The linearized double-stranded DNA molecule of claim 59, wherein the second duplex region comprises first and second read primer binding sites, wherein each first and second read primer sites is further associated with a unique molecule identifier (UMI) and a sample index sequence.

63. The linearized double-stranded DNA molecule of claim 62, wherein the first and second read primer binding sites are orientated outwardly towards the ends of the linearized double-stranded DNA molecule.

64. The linearized double-stranded DNA molecule of claim 62, wherein a first read primer can be used to obtain a sequence read comprising a UMI, sample index, and the original top strand, or portion thereof, of the sample DNA fragment to be sequenced.

65. The linearized double-stranded DNA molecule of claim 62, wherein a second read primer can be used to obtain a sequence read comprising a UMI, sample index, and the original bottom strand, or portion thereof, of the sample DNA fragment to be sequenced.

66. A method for next-generation sequencing of a DNA sample, comprising:
(a) obtaining a DNA sample from a biological source;
(b) fragmenting the DNA sample to obtain a plurality of DNA fragments;
(b) constructing a next-generation sequencing library of DNA fragments by a method of any one of claims 47-57 to generate a plurality of linearized double-stranded DNA molecules, wherein each strand comprises concatemer of top and bottom strands of a DNA fragment; and
(d) determining the sequence of the top and bottom strands of the DNA fragment using next-generation sequencing with read primers that bind to the linearized double-stranded DNA molecule, thereby obtaining the sequence of the DNA molecule.

67. The method of claim 66, wherein the biological sample is blood.

68. The method of claim 66, wherein the biological sample is a sample of tissue from liver, kidney, brain, heart, skin, lung, colon, or pancreas.

69. The method of claim 66, wherein the biological sample a sample of a diseased tissue from liver, kidney, brain, heart, skin, lung, colon, or pancreas.

70. The method of claim 69, wherein the diseased tissue is a proliferative disease.

71. The method of claim 69, wherein the diseased tissue is a tumor.

72. The method of claim 66, wherein the sequencing error rate is similar to a control based on Duplex Sequencing, but wherein the number of reads required is decreased by at least 100-fold.

73. The isolated nucleic acid complex of any one of claims 1-22 for use in a method of methylation sequencing, wherein at least one oligonucleotide is modified to contain methylated cytosine in place of unmethylated cytosine.

74. The isolated nucleic acid complex of any one of claims 1-22 for use in a method of methylation sequencing, wherein each of the first, second, third, and fourth oligonucleotides is modified to contain methylated cytosine in place of unmethylated cytosine.

75. The sequencing adapter of any one of claims 25-38 for use in a method of methylation sequencing, wherein at least one oligonucleotide is modified to contain methylated cytosine in place of unmethylated cytosine.

76. The sequencing adapter of any one of claims 25-38 for use in a method of methylation sequencing, wherein each of the first, second, third, and fourth oligonucleotides is modified to contain methylated cytosine in place of unmethylated cytosine.

77. A method of methylation sequencing of a DNA sample, comprising:
(a) ligating the first and second ends of a sequencing adapter of any one of claims 25-38 to a DNA fragment having opposing top and bottom strands, thereby forming a partially circularized DNA molecule comprising the DNA fragment and the sequencing adapter, wherein the sequencing adapter is modified to contain methylated cytosine in place of unmethylated cytosine; and
(b) synthesizing first and second single-strand DNA molecules by extending the free 3′ ends on the sequencing adapter each using the opposite strand of the partially circularized DNA molecule as a template,
thereby forming a linearized double-stranded DNA molecule, wherein each strand comprises a concatemer of the top and bottom strands of the DNA fragment, wherein the synthesizing step comprises contacting the free 3′ ends with a DNA polymerase and methylated-dCTP along with standard dATP, dGTP and dTTP deoxynucleotides;
(c) deaminating unmethylated cytosines to uracils in the original top strand of the DNA fragment;
(d) determining the sequence of the top and bottom strands by next generation sequencing,
(e) comparing the sequences to infer methylation positions in the original DNA fragment.

78. The method of claim 77, wherein the DNA sample is obtained from a biological sample.

79. The method of claim 78, wherein the biological sample is obtained from liver, kidney, brain, heart, skin, lung, colon, or pancreas tissue, optionally wherein the tissue is diseased.

80. The method of claim 79, wherein the disease is a proliferative disease.

81. The method of claim 79, wherein the disease is a tumor.

82. The method of claim 39, wherein the dsDNA duplex is pre-amplified prior to step (a), the method comprising:
(a) contacting the dsDNA duplex with a first and a second pre-amplification molecule, wherein each of the two pre-amplification molecules comprises a UMI, a sample index, a rolling circle amplification (RCA) primer, and a truncation site;
(b) ligating the first pre-amplification molecule to one first end of the dsDNA duplex and ligating the second pre-amplification molecule to the second end of the dsDNA duplex to produce a pre-amplification dsDNA duplex;
(c) exposing the pre-amplification dsDNA duplex to a DNA polymerase enzyme;
(d) incubating the pre-amplification dsDNA duplex and the DNA polymerase enzyme for a sufficient time to complete RCA; and
(e) removing the RCA primer by cleaving the pre-amplification dsDNA duplex at the truncation site.

83. The method of claim 47, wherein the DNA duplexes to be sequences are pre-amplified prior to step (a), the method comprising:
(a) contacting each of the DNA duplexes to be sequenced with a first and a second pre-amplification molecule, wherein each of the two pre-amplification molecules comprises a UMI, a sample index, a rolling circle amplification (RCA) primer, and a truncation site;
(b) ligating the first pre-amplification molecule to one first end of each of the DNA duplexes to be sequenced and ligating the second pre-amplification molecule to the second end of each the DNA duplexes to be sequenced to produce a plurality of pre-amplification DNA duplexes;
(c) exposing each of the pre-amplification DNA duplexes to a DNA polymerase enzyme;
(d) incubating each of the pre-amplification DNA duplexes and the DNA polymerase enzyme for a sufficient time to complete RCA; and
(e) removing the RCA primer by cleaving each of the pre-amplification DNA duplexes at the truncation site.

84. A method of preparing a next-generation sequencing library, comprising:
(a) blocking the 3′ end of R06 and the 3′ end of R05 from undergoing ligation;
(b) ligating the complex of any one of claims 1-22 to the dsDNA duplex as follows: ligating the 5′ end of R01 to the 3′ end of a first strand of the dsDNA duplex; and ligating the 5′ end of R10 to the 3′ end of a second strand of the dsDNA duplex; thereby forming a circular double-stranded DNA intermediate comprising the target DNA molecule and the complex;
(c) extending a first DNA strand from the 3′ end of R03;
(d) extending a second DNA strand from the 3′ end of R08; and
(e) circularizing each of the first and second DNA strands to form circular, single-stranded sequencing molecules;
(f) introducing a nick into a region between R03 and R08 to form linear, single-stranded sequencing molecules.

85. The method of claim 84, wherein the blocking of step (a) comprising adding a blocking solution.

86. The method of claim 84 or 85, wherein the ligating step of step (b) comprises adding ligase.

87. The method of any one of claims 84-86, wherein the synthesizing of steps (c) and (d) comprise contacting the circular double-stranded DNA intermediate with a polymerase.

88. The method of any one of claims 84-87, wherein the polymerase is a DNA-dependent DNA polymerase.

89. The method of any one of claims 84-88, wherein the polymerase has a strand-displacement activity.

90. The method of any one of claims 84-89, wherein the next-generation sequencing (NGS) is a short-read strategy.

91. A method of preparing a sequencing library, comprising:
(a) obtaining a dsDNA duplex;
(b) treating the dsDNA duplex by duplex repair;
(c) ligating the complex of any one of claims 1-22 to a dsDNA duplex as follows: ligating the 5′ end of R01 to the 3′ end of a first strand of the dsDNA duplex; ligating the 3′ end of R05 to the 5′ end of the first strand of the dsDNA duplex; ligating the 5′ end of R10 to the 3′ end of a second strand of the dsDNA duplex; and ligating the 3′ end of R06 to the 5′ end of the second strand of the dsDNA duplex; thereby forming a circular double-stranded DNA intermediate comprising the target DNA molecule and the complex;
(d) extending a first DNA strand from the 3′ end of R03;
(e) extending a second DNA strand from the 3′ end of R08; and
(f) optionally annealing the first and second DNA strands to form a double-stranded DNA molecule for use in next-generation sequencing (NGS) of the target DNA molecule.

92. A method of preparing a sequencing library comprising a plurality of DNA duplexes to be sequenced:
(a) treating the plurality of DNA duplexes by duplex repair;
(b) ligating the first and second ends of a sequencing adapter of any one of claims 25-36 to a sample DNA fragment having opposing top and bottom strands, thereby forming a partially circularized DNA molecule comprising the DNA fragment and the sequencing adapter; and
(c) synthesizing first and second single-strand DNA molecules by extending the free 3′ ends on the sequencing adapter each using the opposite strand of the partially circularized DNA molecule as a template, thereby forming a linearized double-stranded DNA molecule configured for next generation sequencing, said linearized double-stranded DNA molecule comprising a first double-stranded region comprising the original top strand paired with a copied bottom strand, and a second double-stranded region comprising a copied top strand paired with the original bottom strand,
wherein a plurality of linearized double-stranded DNA molecule each prepared from a different DNA fragment constitute the next-generation sequencing library.


SEQUENCE LISTING (26 sequences)

SEQ ID NO: 1 - DNA, 48 bp, Artificial Sequence
  Synthetic
  misc_feature (47)..(48): linked via phosphorothioate
aatgatacggcgaccaccgagatctacaccttgaacggactgtccact

SEQ ID NO: 2 - DNA, 48 bp, Artificial Sequence
  Synthetic
  misc_feature (47)..(48): linked via phosphorothioate
aatgatacggcgaccaccgagatctacacgagcctactcagtcaacgt

SEQ ID NO: 3 - DNA, 48 bp, Artificial Sequence
  Synthetic
  misc_feature (47)..(48): linked via phosphorothioate
aatgatacggcgaccaccgagatctacacgcttgtaaggcaggttagt

SEQ ID NO: 4 - DNA, 48 bp, Artificial Sequence
  Synthetic
  misc_feature (47)..(48): linked via phosphorothioate
aatgatacggcgaccaccgagatctacaccaagcgtcttacatggtct

SEQ ID NO: 5 - DNA, 43 bp, Artificial Sequence
  Synthetic
  misc_feature (42)..(43): linked via phosphorothioate
caagcagaagacggcatacgagatcaccgagcgttagactact

SEQ ID NO: 6 - DNA, 43 bp, Artificial Sequence
  Synthetic
  misc_feature (42)..(43): linked via phosphorothioate
caagcagaagacggcatacgagatgtgtcgaacacttgacggt

SEQ ID NO: 7 - DNA, 43 bp, Artificial Sequence
  Synthetic
  misc_feature (42)..(43): linked via phosphorothioate
caagcagaagacggcatacgagatctgatcttcagctgactgt

SEQ ID NO: 8 - DNA, 43 bp, Artificial Sequence
  Synthetic
  misc_feature (42)..(43): linked via phosphorothioate
caagcagaagacggcatacgagatgaatctgaggcactgtact

SEQ ID NO: 9 - DNA, 98 bp, Artificial Sequence
  Synthetic
  misc_feature (19)..(21): n is a, c, g, or t
gtggacagtccgttcaagnnnagatcggaagagcgtcgtgtagggaaagagtgtttacatagttatccgctagactctgacgtgttgatcctcgaagc

SEQ ID NO: 10 - DNA, 98 bp, Artificial Sequence
  Synthetic
  misc_feature (19)..(21): n is a, c, g, or t
cgttgactgagtaggctcnnnagatcggaagagcgtcgtgtagggaaagagtgtttacatagttatccgctagactctgacgtgttgatcctcgaagc

SEQ ID NO: 11 - DNA, 99 bp, Artificial Sequence
  Synthetic
  misc_feature (20)..(22): n is a, c, g, or t
ctaacctgccttacaagctnnnagatcggaagagcgtcgtgtagggaaagagtgtttacatagttatccgctagactctgacgtgttgatcctcgaagc

SEQ ID NO: 12 - DNA, 99 bp, Artificial Sequence
  Synthetic
  misc_feature (20)..(22): n is a, c, g, or t
gaccatgtaagacgcttgannnagatcggaagagcgtcgtgtagggaaagagtgtttacatagttatccgctagactctgacgtgttgatcctcgaagc

SEQ ID NO: 13 - DNA, 98 bp, Artificial Sequence
  Synthetic
  misc_feature (19)..(21): n is a, c, g, or t
gtagtctaacgctcggtgnnnagatcggaagagcacacgtctgaactccagtcaccaatctataagttgcttcgaggatcaacacgtcagagtctagc

SEQ ID NO: 14 - DNA, 98 bp, Artificial Sequence
  Synthetic
  misc_feature (19)..(21): n is a, c, g, or t
ccgtcaagtgttcgacacnnnagatcggaagagcacacgtctgaactccagtcaccaatctataagttgcttcgaggatcaacacgtcagagtctagc

SEQ ID NO: 15 - DNA, 99 bp, Artificial Sequence
  Synthetic
  misc_feature (20)..(22): n is a, c, g, or t
cagtcagctgaagatcagtnnnagatcggaagagcacacgtctgaactccagtcaccaatctataagttgcttcgaggatcaacacgtcagagtctagc

SEQ ID NO: 16 - DNA, 99 bp, Artificial Sequence
  Synthetic
  misc_feature (20)..(22): n is a, c, g, or t
gtacagtgcctcagattcannnagatcggaagagcacacgtctgaactccagtcaccaatctataagttgcttcgaggatcaacacgtcagagtctagc

SEQ ID NO: 17 - DNA, 62 bp, Artificial Sequence
  Synthetic
agatcggaagagcgtcgtgtagggaaagagtgtttacatagttatccgctagactctgacgt

SEQ ID NO: 18 - DNA, 62 bp, Artificial Sequence
  Synthetic
agatcggaagagcacacgtctgaactccagtcaccaatctataagttgcttcgaggatcaac

SEQ ID NO: 19 - DNA, 22 bp, Artificial Sequence
  Synthetic
  misc_feature (1)..(3): n is a, c, g, or t
nnncttgaacggactgtccact

SEQ ID NO: 20 - DNA, 22 bp, Artificial Sequence
  Synthetic
  misc_feature (1)..(3): n is a, c, g, or t
nnngagcctactcagtcaacgt

SEQ ID NO: 21 - DNA, 23 bp, Artificial Sequence
  Synthetic
  misc_feature (1)..(3): n is a, c, g, or t
nnnagcttgtaaggcaggttagt

SEQ ID NO: 22 - DNA, 23 bp, Artificial Sequence
  Synthetic
  misc_feature (1)..(3): n is a, c, g, or t
nnntcaagcgtcttacatggtct

SEQ ID NO: 23 - DNA, 22 bp, Artificial Sequence
  Synthetic
  misc_feature (1)..(3): n is a, c, g, or t
nnncaccgagcgttagactact

SEQ ID NO: 24 - DNA, 22 bp, Artificial Sequence
  Synthetic
  misc_feature (1)..(3): n is a, c, g, or t
nnngtgtcgaacacttgacggt

SEQ ID NO: 25 - DNA, 23 bp, Artificial Sequence
  Synthetic
  misc_feature (1)..(3): n is a, c, g, or t
nnnactgatcttcagctgactgt

SEQ ID NO: 26 - DNA, 23 bp, Artificial Sequence
  Synthetic
  misc_feature (1)..(3): n is a, c, g, or t
nnntgaatctgaggcactgtact